(19) United States
(12) Patent Application Publication
(10) Pub. No.: US 2023/0151340 A1
(43) Pub. Date: May 18, 2023

(54) PLASTIC DEGRADING FUSION PROTEINS AND METHODS OF USING THE SAME

(71) Applicants: Alliance for Sustainable Energy, LLC, Golden, CO (US); University of Portsmouth, Portsmouth (GB)

(72) Inventors: Gregg Tyler BECKHAM, Golden, CO (US); Erika Marie ERICKSON, Berkeley, CA (US); John E. MCGEEHAN, Portsmouth (GB)

(21) Appl. No.: 17/921,833

(22) PCT Filed: May 10, 2021
(86) PCT No.: PCT/US2021/031610

§ 371 Date: Oct. 27, 2022

Related U.S. Application Data
(60) Provisional application No. 63/022,784, filed on May 11, 2020.

(51) Int. Cl. C12N 9/18; C08J 11/10; B09B 3/60

(52) U.S. Cl. CPC C12N 9/18; C08J 11/105; B09B 3/60; C08J 2367/02; C07K 2319/00; B09B 2101/75

(57) ABSTRACT

The present disclosure relates to a non-naturally occurring enzyme that includes a first polypeptide that catalyzes the hydrolysis of a polyester to produce mono-(2-hydroxyethyl) terephthalate (MHET), a second polypeptide that catalyzes the cleavage of MHET to produce at least one of terephthalic acid or ethylene glycol, and a third polypeptide that links the first polypeptide with the second polypeptide.

### CROSS-REFERENCE TO RELATED APPLICATIONS

This application claims priority from U.S. Provisional Patent Application No. 63/022,784 filed on May 11, 2020, the contents of which is incorporated herein by reference in their entirety.

### CONTRACTUAL ORIGIN

This invention was made with government support under Contract No. DE-AC36-08G028308 awarded by the Department of Energy. The government has certain rights in the invention.

### SEQUENCE LISTING

The instant application contains a Sequence Listing which has been submitted via EFS-web and is hereby incorporated by reference in its entirety. The ASCII copy created on 7 May2021, is named NREL PCT 20-86_ST25.txt and is 61 kilobytes in size.

### BACKGROUND

Poly (ethylene terephthalate) (PET) is one of the most abundant manmade synthetic polyesters. Crystalline PET is being widely used for production of single-use beverage bottles, clothing, packaging, and carpeting materials. PET resistance to biodegradation due to limited accessibility to ester linkage, and disposal of PET products into the environment pose a serious threat to biosphere, particularly to marine environment. PET can be chemically recycled. However, the extra costs in chemical recycling are not justified when converting PET back to PET. Thus, there remains a need for alternative strategies for recycling/recovering/reusing plastics, for example, polyesters such as PET.

### SUMMARY

An aspect of the present disclosure is a non-naturally occurring enzyme that includes a first polypeptide that catalyzes the hydrolysis of a polyester to produce mono-(2-hydroxyethyl) terephthalate (MHET), a second polypeptide that catalyzes the cleavage of MHET to produce at least one of terephthalic acid or ethylene glycol, and a third polypeptide that links the first polypeptide with the second polypeptide. In some embodiments of the present disclosure, the enzyme may have a sequence identity that is greater than 80% to SEQ ID NO: 36.

In some embodiments of the present disclosure, the enzyme may have a turnover rate of up to 69 s⁻¹. In some embodiments of the present disclosure, the third polypeptide may have about 8 amino acids. In some embodiments of the present disclosure, the enzyme may have a sequence identity that is greater than 80% to SEQ ID NO: 38. In some embodiments of the present disclosure, the enzyme may have a turnover rate of up to 77 s⁻¹. In some embodiments of the present disclosure, the third polypeptide may have about 12 amino acids. In some embodiments of the present disclosure, the enzyme may have a sequence identity that is greater than 80% to SEQ ID NO: 40. In some embodiments of the present disclosure, the enzyme may have a turnover rate of up to 56⁻¹. In some embodiments of the present disclosure, the third polypeptide may have about 20 amino acids.

In some embodiments of the present disclosure, the polyester may include at least one of polyethylene terephthalate (PET), polyglycolic acid, polylactic acid, polycaprolactone, polyhydroxyalkanoate, polyhydroxybutyrate, polyethylene adipate, polybutylene succinate, poly(3-hydroxybutyrate-co-3-hydroxyvalerate), polybutylene terephthalate, polytrimethylene terephthalate, and/or polyethylene naphthlate. In some embodiments of the present disclosure, the third polypeptide may have between 1 and 100 amino acids. In some embodiments of the present disclosure, the third polypeptide may include at least one of glycine, serine, proline, and/or threonine. In some embodiments of the present disclosure, the third polypeptide may covalently link the C-terminus of the second polypeptide to the N-terminus of the first polypeptide.

In some embodiments of the present disclosure, the enzyme may further include a fourth polypeptide capable of catalyzing hydrolysis of a polyester to produce mono-(2-hydroxyethyl) terephthalate (MHET) and a fifth polypeptide, where the fifth polypeptide covalently links the fourth polypeptide with the second polypeptide. In some embodiments of the present disclosure, the enzyme may further include a mutation of at least one of a S to G, a T to L, F, or Y, a E to N, T, D, Q, or G, a R to F, E, T, A, Y, I, S, W, L, V, Q, G, M, or N, a F to P, D, L, A, S, T, E, N, G, or V, a S to A, G, Q, P, E, D, or V, a S to R, A, K, Q, or G, a T to V or L, and/or a F to I. In some embodiments of the present disclosure, the mutation may occur in the second polypeptide.

An aspect of the present disclosure is a genetically modified organism that expresses the enzyme as described herein. In some embodiments of the present disclosure, the organism may include at least one of Pseudomonas putida and/or Escherichia coli.

An aspect of the present disclosure is a method for degrading a polyester, where the method includes contacting an organism as described herein with the polyester.

### BRIEF DESCRIPTION OF THE DRAWINGS

Some embodiments are illustrated in referenced figures of the drawings. It is intended that the embodiments and figures disclosed herein are to be considered illustrative rather than limiting.

FIG. 1 illustrates fusion proteins, according to some embodiments of the present disclosure.

FIG. 2A illustrates a heatmap of synergistic degradation by PETase and MHETase on amorphous PET film over 96 hours at 30° C., according to some embodiments of the present disclosure. Total product release in mM (sum of BHET, MHET, and TPA), x-axis: PETase loading (mg/g PET), y-axis: MHETase loading (mg/g PET).

FIG. 2B illustrates three PETase-MHETase fusion proteins, according to some embodiments of the present disclosure. Linkers composed of glycine (orange) and serine (yellow) residues connecting the C-terminus of MHETase to the N-terminus of PETase.

FIGS. 2C and 2D illustrate a comparison of depolymerization performance of PETase alone, MHETase alone, PETase and MHETase at equimolar loading, and the three fusion proteins on amorphous PET film after 96 h at 30° C., according to some embodiments of the present disclosure. Product release in mM resulting from hydrolysis by FIG. 2C 0.08 mg PETase/g PET or 0.16 mg MHETase/g PET and FIG. 2D 0.25 mg PETase/g PET or 0.5 mg MHETase/g PET. All comparisons are statistically significant with p-values £ 0.0001 based on 2way ANOVA analysis and Tukey's multiple comparisons test.

FIG. 2E illustrates MHET turnover rate by each fusion protein compared to MHETase alone using 250 μM MHET and 5 nM enzyme, according to some embodiments of the present disclosure. Asterisks indicate statistically significant comparisons between MHETase and each chimera enzyme with p-values £ 0.01 (*), 0.001 (**), and 0.0005 (***).

FIG. 3 illustrates SEM images of amorphous PET film after 96 hours of enzyme treatment at 30° C., according to some embodiments of the present disclosure. Digestion conditions represent treatment with no enzyme, treatment with 0.4 mg MHETase/g PET, treatment with 0.4 mg PETase/g PET, simultaneous treatment with 0.4 mg PETase and 0.4 mg MHETase/g PET, and treatment with each fusion protein corresponding to the samples presented in FIG. 2D.

FIG. 4 illustrates a conservation analysis of 6,671 tannase family sequences, according to some embodiments of the present disclosure. Panel A) illustrates conservation scores (relative entropy) of positions in tannase family sequences plotted against the 603 positions in MHETase. A higher relative entropy implies a greater level of amino acid conservation in the site. Panel B) illustrates conservation scores of active-site residues in MHETase within 6 A of the MHET substrate. Conservation scores are shown as percentiles. Arg411, Phe415, and Ser416 are the least conserved active-site positions in the active site and are more variable than 81% of all positions in MHETase. Panel C) illustrates the closest distance between atoms of MHETase active-site residues and the MHET substrate. The molecular coordinates for MHETase bound with MHET are the same as those in the model from which the molecular simulations were started.

FIGS. 5A-5D illustrate amino acid frequencies of active-site positions in MHETase within 6 A of the MHET substrate, according to some embodiments of the present disclosure. The frequency of amino acids for each position was determined from a MAFFT multiple sequence alignment of 6,671 tannase family sequences. The positions are named using Is MHETase numbering, and the red bars indicate the amino acids in Is MHETase.

FIG. 6A illustrates the conservation of Cys positions forming five disulfide bonds in MHETase, according to some embodiments of the present disclosure. Conservation scores are shown as percentiles. Ao FAEB-1 has a 6^{th} disulfide bond between Cys76 and Cys129 which are very variable positions and are less conserved than 98% of positions in multiple sequence alignment.

FIG. 6B illustrates a histogram of Cys occurrence in tannase family sequences showing the rarity of a 6^{th} disulfide bond, according to some embodiments of the present disclosure. Assuming, all Cys form disulfide bonds, less than 8% of tannase family sequences have six disulfide bonds.

FIG. 7A illustrates the sequence identity of 6,671 tannase family sequences retrieved by PSI-BLAST compared to MHETase, according to some embodiments of the present disclosure.

FIGS. 7B and 7C illustrate a conservation analysis of residue positions 131 (FIG. 7B) and 415 (FIG. 7C) (using MHETase numbering), according to some embodiments of the present disclosure. Frequency of each amino acid is based on a multiple sequence alignment of the 6,671 tannase family sequences. The residue found in MHETase at each position is indicated in black.

FIG. 7D illustrates a homology model of the MHET-bound active site within 6 A of the bound substrate comparing MHETase to homology models of the C. thiooxydans and Hydrogenophaga sp. PML113 homologs (generated by SWISS-MODEL), according to some embodiments of the present disclosure.

FIGS. 7E and 7F illustrate the rate of enzymatic turnover of enzymes described herein, according to some embodiments of the present disclosure.

FIGS. 7G through 7J show the initial enzyme reaction velocity as a function of substrate concentration for MHETase, C. thiooxydans, Hydrogenophaga sp. PML113, and the MHETase S131G mutant, respectively, according to some embodiments of the present disclosure. Solid lines represent the Michaelis-Menten kinetic model fit with substrate inhibition.

### REFERENCE NUMBERS

100 fusion protein
110 first polypeptide
120 second polypeptide
130 third polypeptide
140 fourth polypeptide
150 fifth polypeptide

### DETAILED DESCRIPTION

The present disclosure may address one or more of the problems and deficiencies of the prior art discussed above. However, it is contemplated that some embodiments as disclosed herein may prove useful in addressing other problems and deficiencies in a number of technical areas.

Therefore, the embodiments described herein should not necessarily be construed as limited to addressing any of the particular problems or deficiencies discussed herein.

References in the specification to “one embodiment”, “an embodiment”, “an example embodiment”, “some embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

As used herein the term “substantially” is used to indicate that exact values are not necessarily attainable. By way of example, one of ordinary skill in the art will understand that in some chemical reactions 100% conversion of a reactant is possible, yet unlikely. Most of a reactant may be converted to a product and conversion of the reactant may asymptotically approach 100% conversion. So, although from a practical perspective 100% of the reactant is converted, from a technical perspective, a small and sometimes difficult to define amount remains. For this example of a chemical reactant, that amount may be relatively easily defined by the detection limits of the instrument used to test for it. However, in many cases, this amount may not be easily defined, hence the use of the term “substantially”. In some embodiments of the present invention, the term “substantially” is defined as approaching a specific numeric value or target to within 20%, 15%, 10%, 5%, or within 1% of the value or target. In further embodiments of the present invention, the term “substantially” is defined as approaching a specific numeric value or target to within 1%, 0.9%, 0.8%, 0.7%, 0.6%, 0.5%, 0.4%, 0.3%, 0.2%, or 0.10% of the value or target.

As used herein, the term “about” is used to indicate that exact values are not necessarily attainable. Therefore, the term “about” is used to indicate this uncertainty limit. In some embodiments of the present invention, the term “about” is used to indicate an uncertainty limit of less than or equal to ±20%, ±15%, +10%, ±5%, or ±1% of a specific numeric value or target. In some embodiments of the present invention, the term “about” is used to indicate an uncertainty limit of less than or equal to ±1%, ±0.9%, ±0.8%, ±0.7%, ±0.6%, ±0.5%, ±0.4%, ±0.3%, ±0.2%, or ±0.1% of a specific numeric value or target.

A “vector” or “recombinant vector” is a nucleic acid molecule that is used as a tool for manipulating a nucleic acid sequence of choice or for introducing such a nucleic acid sequence into a host cell. A vector may be suitable for use in cloning, sequencing, or otherwise manipulating one or more nucleic acid sequences of choice, such as by expressing or delivering the nucleic acid sequence(s) of choice into a host cell to form a recombinant cell. Such a vector typically contains heterologous nucleic acid sequences not naturally found adjacent to a nucleic acid sequence of choice, although the vector can also contain regulatory nucleic acid sequences (e.g., promoters, untranslated regions) that are naturally found adjacent to the nucleic acid sequences of choice or that are useful for expression of the nucleic acid molecules.

A vector can be either RNA or DNA, either prokaryotic or eukaryotic, and typically is a plasmid. The vector can be maintained as an extrachromosomal element (e.g., a plasmid) or it can be integrated into the chromosome of a recombinant host cell. The entire vector can remain in place within a host cell, or under certain conditions, the plasmid DNA can be deleted, leaving behind the nucleic acid molecule of choice. An integrated nucleic acid molecule can be under chromosomal promoter control, under native or plasmid promoter control, or under a combination of several promoter controls. Single or multiple copies of the nucleic acid molecule can be integrated into the chromosome. A recombinant vector can contain at least one selectable marker.

The term “expression vector” refers to a recombinant vector that is capable of directing the expression of a nucleic acid sequence that has been cloned into it after insertion into a host cell or other (e.g., cell-free) expression system. A nucleic acid sequence is “expressed” when it is transcribed to yield an mRNA sequence. In most cases, this transcript will be translated to yield an amino acid sequence. The cloned gene is usually placed under the control of (i.e., operably linked to) an expression control sequence. The phrase “operatively linked” refers to linking a nucleic acid molecule to an expression control sequence in a manner such that the molecule can be expressed when introduced (i.e., transformed, transduced, transfected, conjugated or conduced) into a host cell.

Vectors and expression vectors may contain one or more regulatory sequences or expression control sequences. Regulatory sequences broadly encompass expression control sequences (e.g., transcription control sequences or translation control sequences), as well as sequences that allow for vector replication in a host cell. Transcription control sequences are sequences that control the initiation, elongation, or termination of transcription. Suitable regulatory sequences include any sequence that can function in a host cell or organism into which the recombinant nucleic acid molecule is to be introduced, including those that control transcription initiation, such as promoter, enhancer, terminator, operator and repressor sequences. Additional regulatory sequences include translation regulatory sequences, origins of replication, and other regulatory sequences that are compatible with the recombinant cell. The expression vectors may contain elements that allow for constitutive expression or inducible expression of the protein or proteins of interest. Numerous inducible and constitutive expression systems are known in the art.

Typically, an expression vector includes at least one nucleic acid molecule of interest operatively linked to one or more expression control sequences (e.g., transcription control sequences or translation control sequences). In one aspect, an expression vector may comprise a nucleic acid encoding a recombinant polypeptide, as described herein, operably linked to at least one regulatory sequence. It should be understood that the design of the expression vector may depend on such factors as the choice of the host cell to be transformed and/or the type of polypeptide to be expressed. As used herein, a “non-natural” polypeptide is synonymous with a “recombinant” polypeptide.

Expression and recombinant vectors may contain a selectable marker, a gene encoding a protein necessary for survival or growth of a host cell transformed with the vector. The presence of this gene allows growth of only those host cells that express the vector when grown in the appropriate selective media. Typical selection genes encode proteins that confer resistance to antibiotics or other toxic substances, complement auxotrophic deficiencies, or supply critical nutrients not available from a particular media. Markers may be an inducible or non-inducible gene and will generally allow for positive selection. Non-limiting examples of selectable markers include the ampicillin resistance marker (i.e., beta-lactamase), tetracycline resistance marker, neomycin/kanamycin resistance marker (i.e., neomycin phosphotransferase), dihydrofolate reductase, glutamine synthetase, and the like. The choice of the proper selectable marker will depend on the host cell, and appropriate markers for different hosts as understood by those of skill in the art.

Suitable expression vectors may include (or may be derived from) plasmid vectors that are well known in the art, such as those commonly available from commercial sources. Vectors can contain one or more replication and inheritance systems for cloning or expression, one or more markers for selection in the host, and one or more expression cassettes. The inserted coding sequences can be synthesized by standard methods, isolated from natural sources, or prepared as hybrids. Ligation of the coding sequences to transcriptional regulatory elements or to other amino acid encoding sequences can be carried out using established methods. A large number of vectors, including bacterial, yeast, and mammalian vectors, have been described for replication and/or expression in various host cells or cell-free systems, and may be used with the sequences described herein for simple cloning or protein expression.

Nucleic acids referred to herein as “isolated” are nucleic acids that have been removed from their natural milieu or separated away from the nucleic acids of the genomic DNA or cellular RNA of their source of origin (e.g., as it exists in cells or in a mixture of nucleic acids such as a library), and may have undergone further processing. Isolated nucleic acids include nucleic acids obtained by methods described herein, similar methods or other suitable methods, including essentially pure nucleic acids, nucleic acids produced by chemical synthesis, by combinations of biological and chemical methods, and recombinant nucleic acids that are isolated.

Nucleic acids referred to herein as “recombinant” are nucleic acids which have been produced by recombinant DNA methodology, including those nucleic acids that are generated by procedures that rely upon a method of artificial replication, such as the polymerase chain reaction (PCR) and/or cloning or assembling into a vector using restriction enzymes.

Recombinant nucleic acids also include those that result from recombination events that occur through the natural mechanisms of cells but are selected for after the introduction to the cells of nucleic acids designed to allow or make probable a desired recombination event. Portions of isolated nucleic acids that code for polypeptides having a certain function can be identified and isolated by, for example, the method disclosed in U.S. Pat. No. 4,952,501.

A nucleic acid molecule or polynucleotide can include a naturally occurring nucleic acid molecule that has been isolated from its natural source or produced using recombinant DNA technology (e.g., polymerase chain reaction (PCR) amplification, cloning) or chemical synthesis. Isolated nucleic acid molecules can include, for example, genes, natural allelic variants of genes, coding regions or portions thereof, and coding and/or regulatory regions modified by nucleotide insertions, deletions, substitutions, and/or inversions in a manner such that the modifications do not substantially interfere with the nucleic acid molecule's ability to encode a polypeptide or to form stable hybrids under stringent conditions with natural gene isolates. An isolated nucleic acid molecule can include degeneracies. As used herein, nucleotide degeneracy refers to the phenomenon that one amino acid can be encoded by different nucleotide codons. Thus, the nucleic acid sequence of a nucleic acid molecule that encodes a protein or polypeptide can vary due to degeneracies.

Unless so specified, a nucleic acid molecule is not required to encode a protein having enzyme activity. A nucleic acid molecule can encode a truncated, mutated, or inactive protein, for example. In addition, nucleic acid molecules may also be useful as probes and primers for the identification, isolation and/or purification of other nucleic acid molecules, independent of a protein-encoding function.

Suitable nucleic acids include fragments or variants that encode a functional enzyme. For example, a fragment can comprise the minimum nucleotides required to encode a functional enzyme. Nucleic acid variants include nucleic acids with one or more nucleotide additions, deletions, substitutions, including transitions and transversions, insertion, or modifications (e.g., via RNA or DNA analogs). Alterations may occur at the 5′ or 3′ terminal positions of the reference nucleotide sequence or anywhere between those terminal positions, interspersed either individually among the nucleotides in the reference sequence or in one or more contiguous groups within the reference sequence.

Embodiments of the nucleic acids include those that encode the polypeptides that function as an O-dealkylase or a reductase or functional equivalents thereof. A functional equivalent includes fragments or variants of these that exhibit the ability to function as an O-dealkylase or a reductase. As a result of the degeneracy of the genetic code, many nucleic acid sequences can encode a given polypeptide with a particular enzymatic activity. Such functionally equivalent variants are contemplated herein.

Nucleic acids may be derived from a variety of sources including DNA, cDNA, synthetic DNA, synthetic RNA, or combinations thereof. Such sequences may comprise genomic DNA, which may or may not include naturally occurring introns. Moreover, such genomic DNA may be obtained in association with promoter regions or poly (A) sequences. The sequences, genomic DNA, or cDNA may be obtained in any of several ways. Genomic DNA can be extracted and purified from suitable cells by means well known in the art. Alternatively, mRNA can be isolated from a cell and used to produce cDNA by reverse transcription or other means.

Also disclosed herein are recombinant vectors, including expression vectors, containing nucleic acids encoding polypeptides and/or enzymes. A “recombinant vector” is a nucleic acid molecule that is used as a tool for manipulating a nucleic acid sequence of choice or for introducing such a nucleic acid sequence into a host cell. A recombinant vector may be suitable for use in cloning, assembling, sequencing, or otherwise manipulating the nucleic acid sequence of choice, such as by expressing or delivering the nucleic acid sequence of choice into a host cell to form a recombinant cell. Such a vector typically contains heterologous nucleic acid sequences not naturally found adjacent to a nucleic acid sequence of choice, although the vector can also contain regulatory nucleic acid sequences (e.g., promoters, untranslated regions) that are naturally found adjacent to the nucleic acid sequences of choice or that are useful for expression of the nucleic acid molecules.

The nucleic acids described herein may be used in methods for production of enzymes and enzyme cocktails through incorporation into cells, tissues, or organisms. In some embodiments, a nucleic acid may be incorporated into a vector for expression in suitable host cells. The vector may then be introduced into one or more host cells by any method known in the art. One method to produce an encoded protein includes transforming a host cell with one or more recombinant nucleic acids (such as expression vectors) to form a recombinant cell. The term “transformation” is generally used herein to refer to any method by which an exogenous nucleic acid molecule (i.e., a recombinant nucleic acid molecule) can be inserted into a cell but can be used interchangeably with the term “transfection.”

Non-limiting examples of suitable host cells include cells from microorganisms such as bacteria, yeast, fungi, and filamentous fungi. Exemplary microorganisms include, but are not limited to, bacteria such as E. coli; bacteria from the genera Pseudomonas (e.g., P. putida or P. fluorescens), Bacillus (e.g., B. subtilis, B. megaterium or B. brevis), Caulobacter (e.g., C. crescentus), Lactoccocus (e.g., L. lactis), Streptomyces (e.g., S. coelicolor), Streptococcus (e.g., S. lividans), and Corynybacterium (e.g., C. glutamicum); fungi from the genera Trichoderma (e.g., T. reesei, T. viride, T. koningii, or T. harzianum), Penicillium (e.g., P. funiculosum), Humicola (e.g., H. insolens), Chrysosporium (e.g., C. lucknowense), Gliocladium, Aspergillus (e.g., A. niger, A. nidulans, A. awamori, or A. aculeatus), Fusarium, Neurospora, Hypocrea (e.g., H. jecorina), and Emericella; yeasts from the genera Saccharomyces (e.g., S. cerevisiae), Pichia (e.g., P. pastoris), or Kluyveromyces (e.g., K. lactis). Cells from plants such as Arabidopsis, barley, citrus, cotton, maize, poplar, rice, soybean, sugarcane, wheat, switch grass, alfalfa, miscanthus, and trees such as hardwoods and softwoods are also contemplated herein as host cells.

Host cells can be transformed, transfected, or infected as appropriate by any suitable method including electroporation, calcium chloride-, lithium chloride-, lithium acetate/polyene glycol-calcium, phosphate-, DEAE-dextran-, liposome-mediated DNA uptake, spheroplasting, injection, microinjection, microprojectile bombardment, phage infection, viral infection, or other established methods. Alternatively, vectors containing the nucleic acids of interest can be transcribed in vitro, and the resulting RNA introduced into the host cell by well-known methods, for example, by injection. Exemplary embodiments include a host cell or population of cells expressing one or more nucleic acid molecules or expression vectors described herein (for example, a genetically modified microorganism). The cells into which nucleic acids have been introduced as described above also include the progeny of such cells.

Vectors may be introduced into host cells such as those from bacteria or fungi by direct transformation, in which DNA is mixed with the cells and taken up without any additional manipulation, by conjugation, electroporation, or other means known in the art. Expression vectors may be expressed by bacteria or fungi or other host cells episomally or the gene of interest may be inserted into the chromosome of the host cell to produce cells that stably express the gene with or without the need for selective pressure. For example, expression cassettes may be targeted to neutral chromosomal sites by recombination.

Host cells carrying an expression vector (i.e., transformants or clones) may be selected using markers depending on the mode of the vector construction. The marker may be on the same or a different DNA molecule. In prokaryotic hosts, the transformant may be selected, for example, by resistance to ampicillin, tetracycline or other antibiotics. Production of a particular product based on temperature sensitivity may also serve as an appropriate marker.

Host cells may be cultured in an appropriate fermentation medium. An appropriate, or effective, fermentation medium refers to any medium in which a host cell, including a genetically modified microorganism, when cultured, is capable of growing or expressing the polypeptides described herein. Such a medium is typically an aqueous medium comprising assimilable carbon, nitrogen, and phosphate sources, but can also include appropriate salts, minerals, metals and other nutrients. Microorganisms and other cells can be cultured in conventional fermentation bioreactors and by any fermentation process, including batch, fed-batch, cell recycle, and continuous fermentation. The pH of the fermentation medium is regulated to a pH suitable for growth of the particular organism. Culture media and conditions for various host cells are known in the art. A wide range of media for culturing bacteria or fungi, for example, are available from ATCC. Exemplary culture/fermentation conditions and reagents are provided in the Examples that follow. Media may be supplemented with aromatic substrates like guaiacol, guaethol or anisole for dealkylation reactions.

As used herein, the terms “protein” and “polypeptide” are synonymous. “Peptides” are defined as fragments or portions of polypeptides, preferably fragments or portions having at least one functional activity as the complete polypeptide sequence. “Isolated” proteins or polypeptides are proteins or polypeptides purified to a state beyond that in which they exist in cells. In certain embodiments, they may be at least 10% pure; in others, they may be substantially purified to 80% or 90% purity or greater. Isolated proteins or polypeptides include essentially pure proteins or polypeptides, proteins or polypeptides produced by chemical synthesis or by combinations of biological and chemical methods, and recombinant proteins or polypeptides that are isolated. Proteins or polypeptides referred to herein as “recombinant” are proteins or polypeptides produced by the expression of recombinant nucleic acids.

Polypeptides may be retrieved, obtained, or used in “substantially pure” form, a purity that allows for the effective use of the protein in any method described herein or known in the art. For a protein to be most useful in any of the methods described herein or in any method utilizing enzymes of the types described herein, it is most often substantially free of contaminants, other proteins and/or chemicals that might interfere or that would interfere with its use in the method (e.g., that might interfere with enzyme activity), or that at least would be undesirable for inclusion with a protein. In an embodiment, a non-naturally occurring enzyme may also be referred to as a recombinant protein Among other things, the present disclosure relates to fusion proteins, chimeric enzymes, for depolymerizing plastics, for example, polyethylene terephthalate (PET). As described herein, fusion proteins are disclosed having at least a two-enzyme system of a first enzyme (i.e., a first polypeptide) for deconstructing PET (i.e., a PETase) to its constituent monomers, including mono-(2-hydroxyethyl) terephthalate (MHET), and a second enzyme (i.e., a second polypeptide), a MHETase, which cleaves the MHET to yield terephthalic acid (TPA) and ethylene glycol (EG).

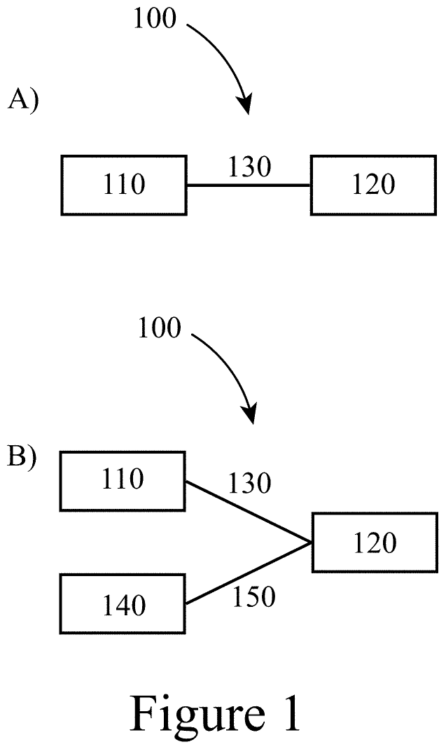

FIG. 1 illustrates fusion proteins 100, according to some embodiments of the present disclosure. Referring to Panel A), a fusion protein 100 may include a first polypeptide 110, for example an enzyme capable of degrading a polyester to an intermediate, smaller molecular weight molecule. This first polypeptide 100 may be covalently linked to a second polypeptide 120, for example an enzyme capable of further degrading (e.g., cleaving) the intermediate to even smaller molecular weight components. The first polypeptide 100 may be covalently linked to the second polypeptide by a third polypeptide 130, for example a flexible chain of amino acids. Panel B) illustrates that, according to some embodiments of the present disclosure, a fusion protein may include three or more catalytically active polypeptides covalently linked together by one or more linker polypeptides. In some embodiments of the present disclosure, a linker polypeptide may have between 1 and 100 amino acids, or between 20 and 100 amino acids, or between 10 and 50 amino acids. Panel B) illustrates that in some embodiments of the present disclosure, a fusion protein 100 may include two linking molecules (130 and 150), linking two enzymes (110 and 140) to a third enzyme 120. In this example, two enzymes (110 and 140) capable of degrading a polyester to an intermediate may be covalently linked by two separate flexible amino acid chains (130 and 150, respectively) to a polypeptide 120 capable of further degrading (e.g., cleaving) the intermediate to even smaller molecular weight components. In some embodiments of the present disclosure, the three or more enzymes may be covalently bound in linear fashion along an unbranched polypeptide chain. For example, using the reference numbers of Panel B) of FIG. 1: 110 to 130 to 140 to 150 to 120.

In some embodiments of the present disclosure, a fusion protein 100 may include a first polypeptide 110 capable of catalyzing hydrolysis of a polyester to produce a first intermediate covalently linked to a second polypeptide 120 capable of catalyzing cleavage of the first intermediate to produce smaller molecular weight compounds. The first polypeptide 110 may be covalently linked to the second polypeptide 120 by a third polypeptide, for example a flexible chain of amino acids. For the example where the polyester includes polyethylene terephthalate (PET), a first polypeptide 110 capable of catalyzing hydrolysis of the PET to produce at least mono-(2-hydroxyethyl) terephthalate (MHET) is referred to herein as a PETase and the second polypeptide 120 capable of further degrading the MHET to at least one of terephthalic acid and/or ethylene glycol is referred to herein as a MHETase.

In some embodiments of the present disclosure, a fusion protein 100 may be capable of degrading a plastic such as a polyester to smaller molecular weight compounds that may be reused to produce valuable materials. Examples of polyesters that may be degraded using the enzymes, organisms, and methods described herein include at least one of polyethylene terephthalate (PET), polyglycolic acid, polylactic acid, polycaprolactone, polyhydroxyalkanoate, polyhydroxybutyrate, polyethylene adipate, polybutylene succinate, poly(3-hydroxybutyrate-co-3-hydroxyvalerate), polybutylene terephthalate, polytrimethylene terephthalate, and/or polyethylene naphthlate.

In some embodiments of the present disclosure, at least one of the first polypeptide (e.g., PETase) and/or the second polypeptide (e.g., MHETase) may be derived from at least one of a bacterium and/or a fungus. In some embodiments of the present disclosure, the first polypeptide and/or the second polypeptide may be derived from a fungus such as Fusarium solani. In some embodiments of the present disclosure, the first polypeptide and/or the second polypeptide may be derived from a bacterium from a family that includes at least one of Comamonadaceae and/or Nocardiopsaceae. In some embodiments of the present disclosure, the first polypeptide and/or the second polypeptide may be derived from a bacterium from a genus that includes at least one of Ideonella, Comamonas, Hydrogenophaga, and/or Thermobifida. In some embodiments of the present disclosure, the first polypeptide and/or the second polypeptide may be derived from a bacterium that includes at least one of Ideonella sakaiensis, and/or Comamonas thiooxydans.

In some embodiments of the present disclosure, a third polypeptide 130 that covalently links a first polypeptide 110 to a second polypeptide may include between 1 amino acid and 100 amino acids, inclusively. In an embodiment, a third peptide is from about 10 to about 50 amino acids. In an embodiment, a third peptide is from about 20 to about 50 amino acids. In an embodiment, a third peptide is from about 10 to about 80 amino acids. In an embodiment, a third peptide is from about 20 to about 80 amino acids. In an embodiment, a third peptide is from about 10 to about 90 amino acids. In an embodiment, a third peptide is from about 20 to about 90 amino acids. In some embodiments of the present disclosure a third polypeptide 130, i.e., a linking protein chain, may include at least 2 amino acids, at least 5 amino acids, at least 8 amino acids, at least 11 amino acids, at least 14 amino acids, at least 17 amino acids, or at least 20 amino acids. In some embodiments of the present disclosure a third polypeptide 130, i.e., a linking protein chain, may include up to 25 amino acids, up to 50 amino acids, up to 75 amino acids, or up to 100 amino acids. A linking protein may be constructed of amino acids that include, among others, at least one of glycine, serine, proline, and/or threonine. In some embodiments of the present disclosure, a third polypeptide 130 (i.e., a linking protein chain) may covalently link the C-terminus of the second polypeptide 120 to the N-terminus of the first polypeptide 110. In some embodiments of the present disclosure, a first polypeptide 110 may be covalently linked to a third polypeptide 130 by a maleimide crosslinker, provided each polypeptide has a sulfhydryl group (—SH). Examples of a maleimide include bis-maleimidoethane and 1,4-di(maleimido)butane.

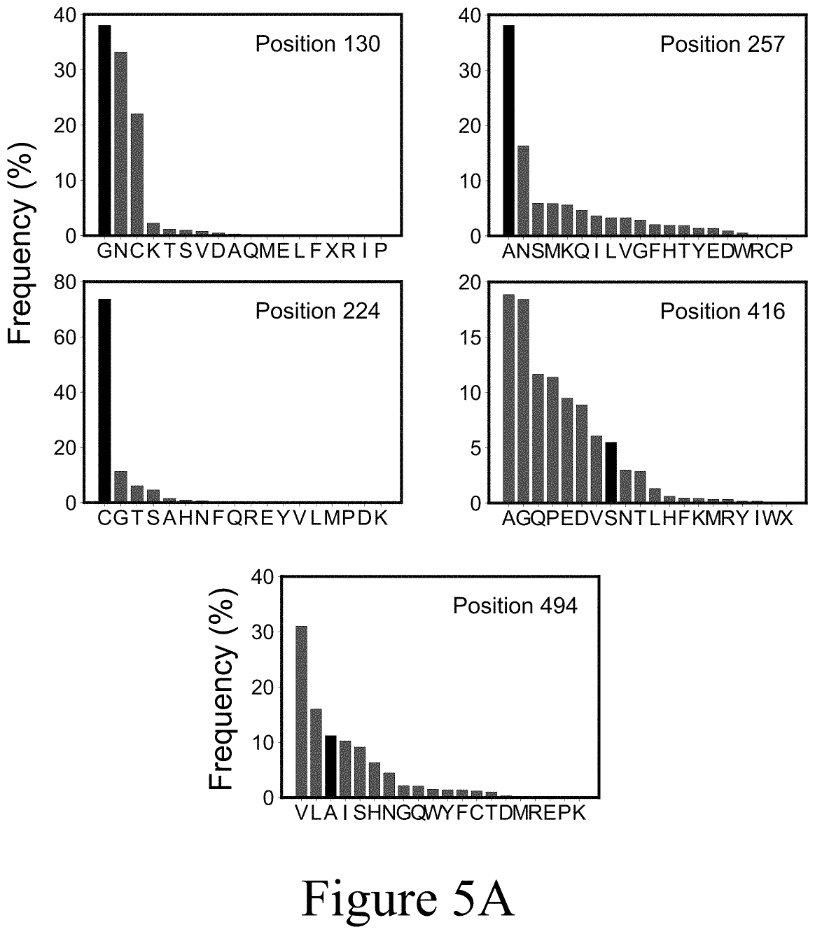

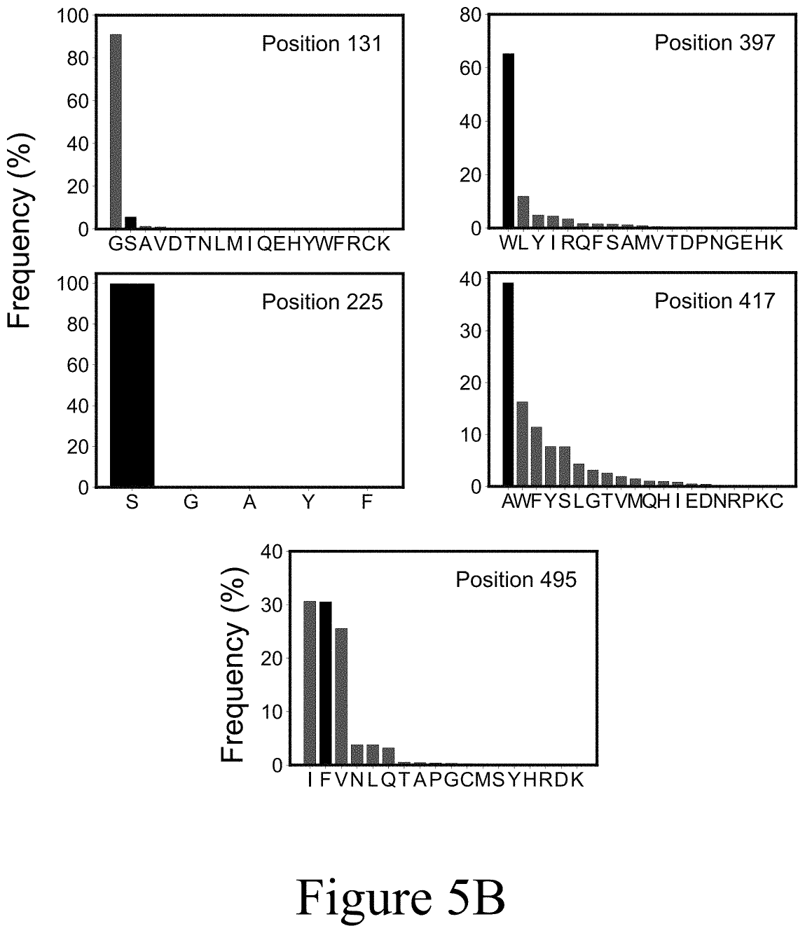

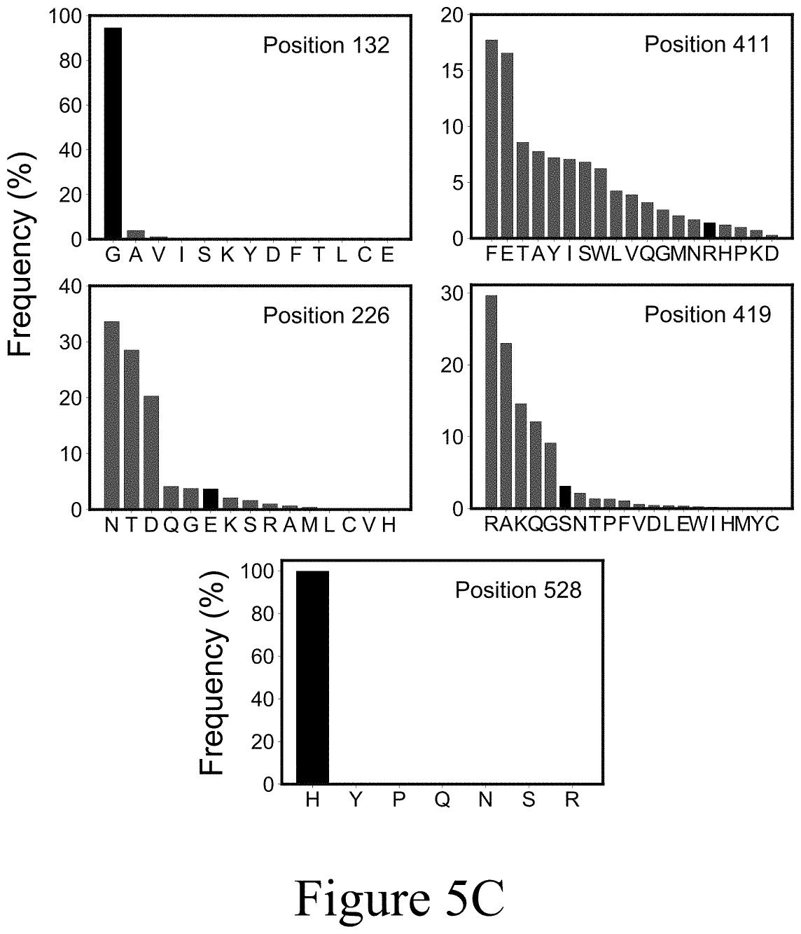

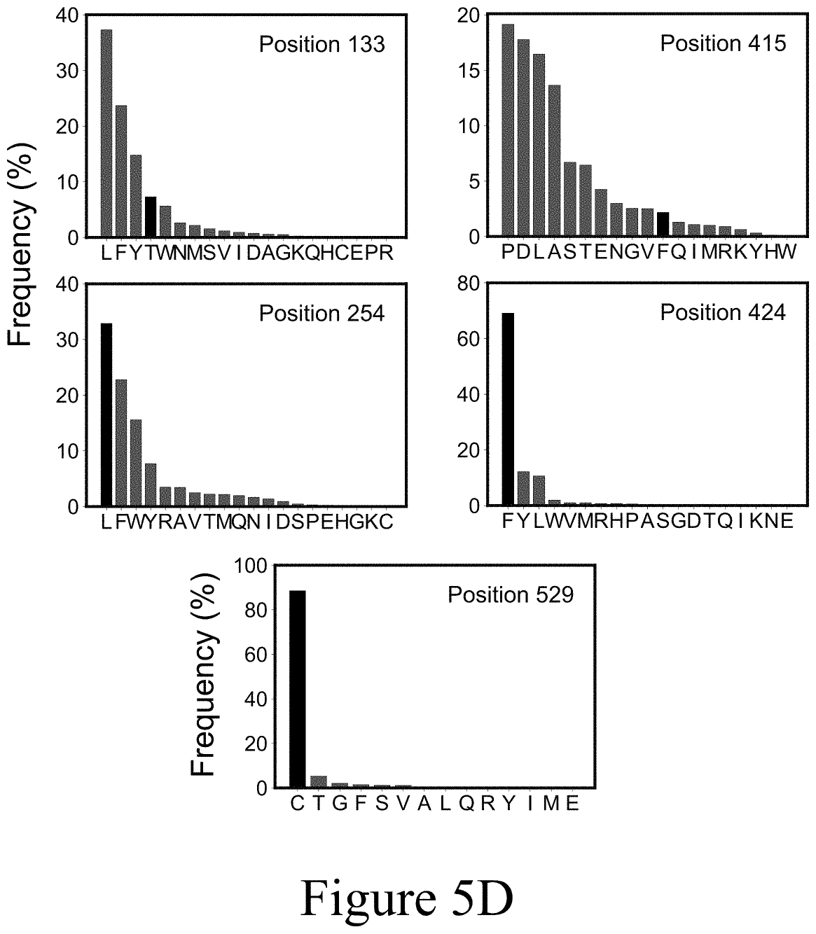

In some embodiments of the present disclosure, at least one of the first polypeptide 110 and/or the second polypeptide 120 may include a mutation to at least one amino acid, resulting in improved catalytic activity by the mutated polypeptide, as described herein. In some embodiments of the present disclosure at least one of the amino acids of a MHETase as described herein, may be mutated at least one of the following locations along the MHETase polypeptide: 131 (S to G), 133 (T to L, F, or Y), 226 (E to N, T, D, Q, or G), 411 (R to F, E, T, A, Y, I, S, W, L, V, Q, G, M, or n), 415 (F to P, D, L, A, S, T, E, N, G, or V), 416 (S to A, G, Q, P, E, D, or V), 419 (S to R, A, K, Q, or G), 494 (a TO V or L), or 495 (F to I). (See FIGS. 5A through 5D.) In some embodiments of the present disclosure, a fusion protein may also include a secretion signal peptide.

In an embodiment, additional enzymes are contemplated herein that at least 80% sequence identity to the enzymes disclosed herein. In other embodiments, additional enzymes are contemplated herein that at least 85%, 90%, 95%, 98%, 99%, and up to 100% sequence identity to the enzymes disclosed herein.

As described herein, an organism may be genetically modified to manufacture the fusion proteins described herein. In some embodiments of the present disclosure, an organism for producing a fusion protein may include a bacterium such as at least one of a Pseudomonas putida and/or Escherichia coli. Further, as described herein, a plastic (e.g., PET) may be degraded to smaller molecular weight compounds by mixing and/or contacting at least one of the fusion proteins and/or organisms producing the fusion proteins with the plastic, where the mixing/contacting results in the degrading of the plastic to smaller molecular weight components.

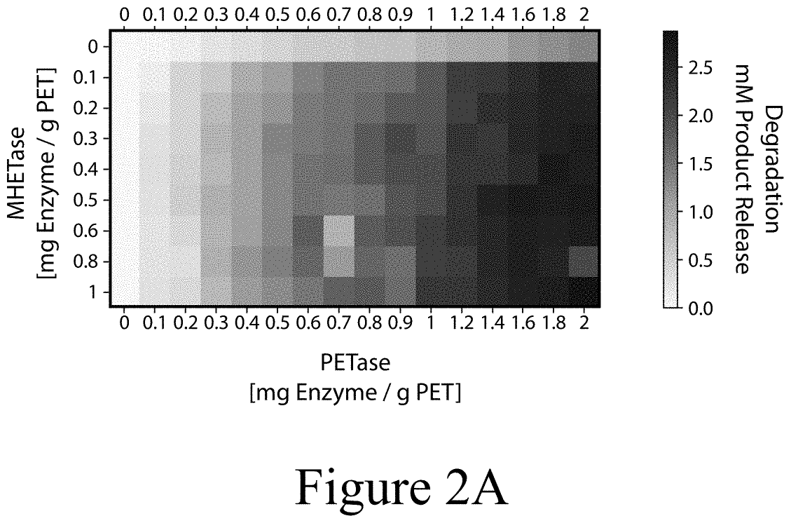

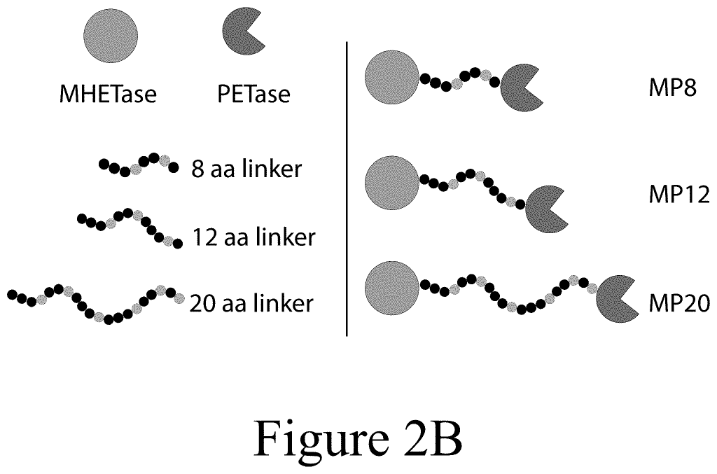

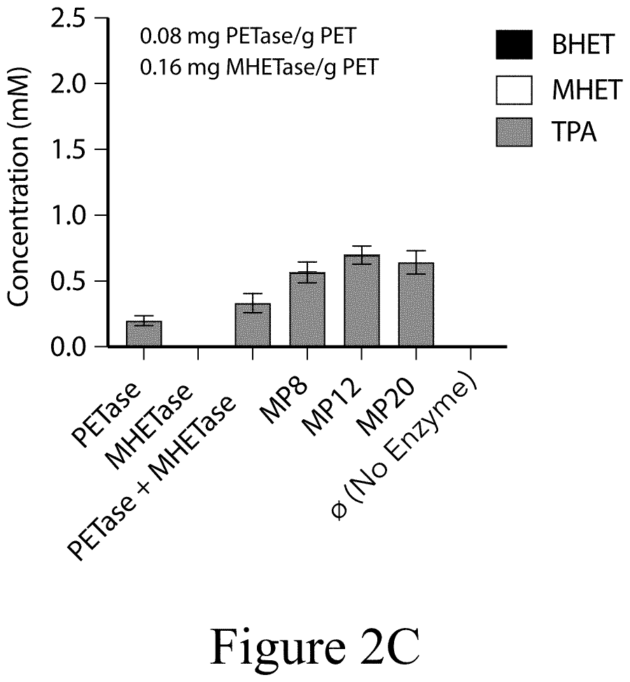

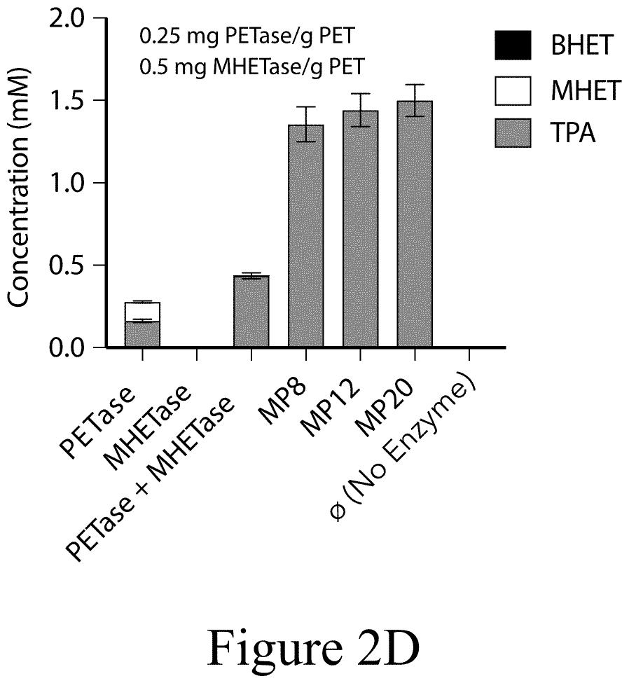

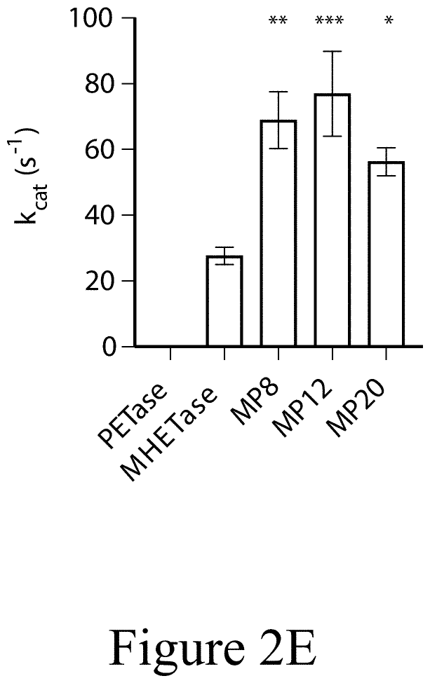

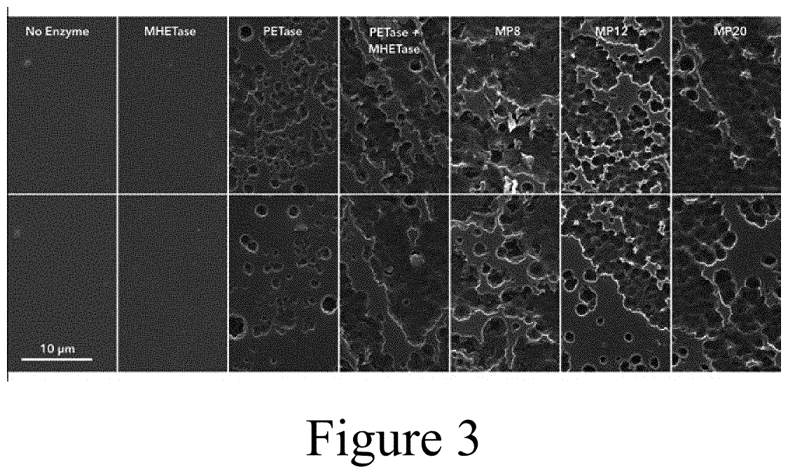

As shown herein, fusion proteins (i.e., chimeric proteins) of MHETase and PETase can improve PET degradation and MHET hydrolysis rates. As described below in more detail, in view of the synergistic relationship between PETase and MHETase on amorphous PET, the relationship between the proximity of the two enzymes and hydrolytic activity was examined. Chimeric proteins covalently linking the C-terminus of MHETase to the N-terminus of PETase using flexible glycine-serine linkers of 8, 12, and 20 total glycine and serine residues were generated and tested for degradation of amorphous PET (see FIG. 2B). Varying linker lengths were explored to understand the effect of increased mobility between the two domains. Furthermore, for comparison to the fusion protein, two loadings of the individual, non-fused enzymes were compared—the lower loading corresponding to approximately 0.08 mg PETase/g PET and 0.16 mg MHETase/g PET, and the higher enzyme loading corresponding to 0.25 mg PETase/g PET and 0.5 mg MHETase/g PET (see FIGS. 2C and 2D). At both loadings, when comparing the extent of degradation achieved by PETase alone, MHETase alone, and an equimolar mix of PETase and MHETase, the fusion proteins outperformed PETase, as well as the mixed reaction containing both PETase and MHETase. Furthermore, the fusion proteins demonstrated a higher catalytic activity on MHET (see FIG. 2E). Fusion proteins linking the C-terminus of PETase to the N-terminus of MHETase did not successfully express protein (see FIG. 2B). SEM analysis of digested amorphous PET film confirms degradation of PET by the fusion proteins described herein (see FIG. 3).

In addition, as shown herein, PETase and MHETase act synergistically during PET depolymerization. While MHET is susceptible to hydrolysis by a number of PET-degrading cutinases, I. sakaiensis favors the action of two enzymes for PET degradation to liberate TPA and EG. To better understand this two-enzyme system, the extent of hydrolysis was measured of a commercial amorphous PET substrate over 96 hours at 30° C. using PETase and MHETase at varying concentrations (see FIG. 2A and Table 1). As expected, MHETase alone has no activity on PET film. Over the range of enzyme loadings tested (between 0 and 2.0 mg enzyme/g PET), degradation by PETase alone, as determined by concentration of product released (the sum of BHET, MHET, and TPA), scaled with enzyme loading and then plateaued. An optimal ratio of PETase:MHETase loading was observed at 0.4 mg each enzyme/g PET, corresponding to an approximately 2:1 molar ratio (see FIG. 2A). The presence of MHETase in concentrations ranging between 0.2 and 0.4 mg enzyme/g PET in the reaction enhanced degradation as compared to that observed for the same loading of PETase without MHETase. At higher levels of MHETase loading, however, degradation was negatively impacted and resulted in less product release than in reactions containing the same loading of PETase with MHETase.

TABLE 1
Synergistic degradation of amorphous PET film over 96 hours at 30° C.

| | | | | | Sum total of |
|---|---|---|---|---|---|
| PETase loading | MHETase loading | TPA | MHET | BHET | product release |
| (mg enzyme/g PET) | (mg enzyme/g PET) | (mM) | (mM) | (mM) | (mM) |
| 0 | 0 | 0.00 ± 0.00 | 0.00 ± 0.00 | 0.00 ± 0.00 | 0.00 ± 0.00 |
| 0 | 0.4 | 0.00 ± 0.00 | 0.00 ± 0.00 | 0.00 ± 0.00 | 0.00 ± 0.00 |
| 0 | 0.8 | 0.00 ± 0.00 | 0.00 ± 0.00 | 0.00 ± 0.00 | 0.00 ± 0.00 |
| 0.2 | 0.2 | 2.42 ± 0.11 | 0.00 ± 0.00 | 0.00 ± 0.00 | 2.43 ± 0.12 |
| 0.2 | 0.6 | 1.47 ± 0.21 | 0.00 ± 0.00 | 0.00 ± 0.00 | 1.47 ± 0.21 |
| 0.2 | 1.0 | 0.10 ± 0.02 | 0.00 ± 0.00 | 0.00 ± 0.00 | 0.10 ± 0.02 |
| 0.4 | 0 | 0.94 ± 0.09 | 1.15 ± 0.13 | 0.00 ± 0.00 | 2.08 ± 0.22 |
| 0.4 | 0.4 | 3.50 ± 0.25 | 0.00 ± 0.00 | 0.01 ± 0.01 | 3.50 ± 0.26 |
| 0.4 | 0.8 | 1.31 ± 0.18 | 0.00 ± 0.00 | 0.00 ± 0.00 | 1.31 ± 0.18 |
| 0.6 | 0.2 | 2.34 ± 0.03 | 0.02 ± 0.03 | 0.01 ± 0.00 | 2.37 ± 0.07 |
| 0.6 | 0.6 | 1.74 ± 0.05 | 0.00 ± 0.00 | 0.01 ± 0.00 | 1.75 ± 0.05 |
| 0.6 | 1.0 | 1.05 ± 0.26 | 0.00 ± 0.00 | 0.00 ± 0.00 | 1.05 ± 0.27 |
| 0.8 | 0 | 1.47 ± 0.04 | 0.58 ± 0.04 | 0.00 ± 0.00 | 2.05 ± 0.08 |
| 0.8 | 0.4 | 2.53 ± 0.19 | 0.03 ± 0.06 | 0.00 ± 0.00 | 2.56 ± 0.25 |
| 0.8 | 0.8 | 1.31 ± 0.22 | 0.00 ± 0.00 | 0.00 ± 0.00 | 1.31 ± 0.22 |
| 1.0 | 0.2 | 2.62 ± 0.17 | 0.07 ± 0.12 | 0.00 ± 0.00 | 2.69 ± 0.29 |
| 1.0 | 0.6 | 2.52 ± 0.33 | 0.00 ± 0.00 | 0.00 ± 0.00 | 2.53 ± 0.33 |
| 1.0 | 1.0 | 1.61 ± 0.21 | 0.00 ± 0.00 | 0.00 ± 0.00 | 1.61 ± 0.21 |
| 1.2 | 0 | 1.30 ± 0.03 | 0.70 ± 0.01 | 0.00 ± 0.00 | 2.00 ± 0.03 |
| 1.2 | 0.4 | 2.57 ± 0.13 | 0.05 ± 0.09 | 0.00 ± 0.00 | 2.62 ± 0.22 |
| 1.2 | 0.8 | 1.47 ± 0.11 | 0.00 ± 0.00 | 0.00 ± 0.00 | 1.47 ± 0.11 |
| 1.4 | 0.2 | 2.39 ± 0.21 | 0.01 ± 0.03 | 0.00 ± 0.00 | 2.40 ± 0.23 |
| 1.4 | 0.6 | 1.67 ± 0.40 | 0.00 ± 0.00 | 0.00 ± 0.00 | 1.67 ± 0.40 |
| 1.4 | 1.0 | 1.84 ± 0.15 | 0.00 ± 0.00 | 0.00 ± 0.00 | 1.84 ± 0.15 |
| 1.6 | 0 | 1.74 ± 0.07 | 0.69 ± 0.06 | 0.00 ± 0.00 | 2.43 ± 0.14 |
| 1.6 | 0.4 | 2.80 ± 0.13 | 0.00 ± 0.00 | 0.00 ± 0.00 | 2.81 ± 0.13 |
| 1.6 | 0.8 | 2.21 ± 0.09 | 0.00 ± 0.00 | 0.00 ± 0.00 | 2.21 ± 0.09 |
| 1.8 | 0.2 | 2.12 ± 0.17 | 0.07 ± 0.06 | 0.00 ± 0.00 | 2.19 ± 0.23 |
| 1.8 | 0.6 | 2.23 ± 0.26 | 0.00 ± 0.00 | 0.01 ± 0.00 | 2.24 ± 0.26 |
| 1.8 | 1.0 | 1.42 ± 0.11 | 0.00 ± 0.00 | 0.01 ± 0.00 | 1.43 ± 0.11 |
| 2.0 | 0 | 1.84 ± 0.25 | 0.21 ± 0.04 | 0.00 ± 0.00 | 2.05 ± 0.30 |
| 2.0 | 0.4 | 2.32 ± 0.59 | 0.06 ± 0.05 | 0.00 ± 0.00 | 2.38 ± 0.64 |
| 2.0 | 0.8 | 1.55 ± 0.29 | 0.00 ± 0.00 | 0.01 ± 0.00 | 1.56 ± 0.29 |

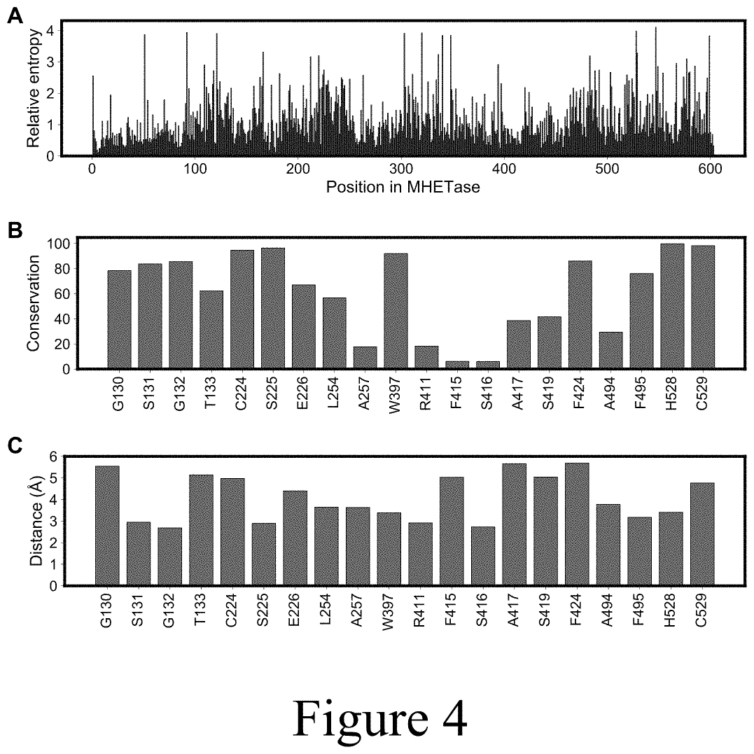

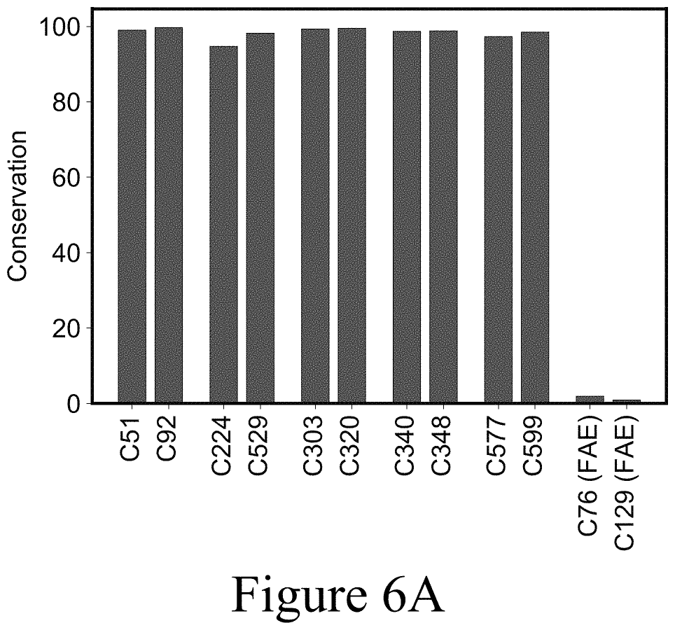

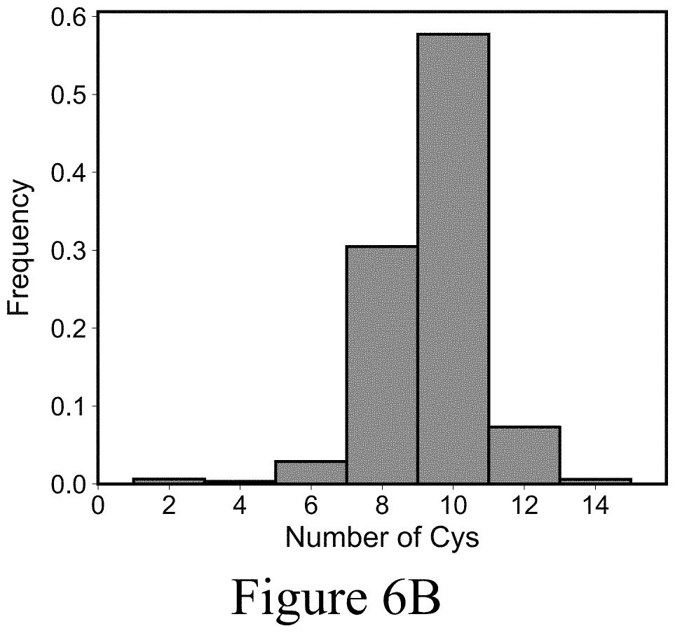

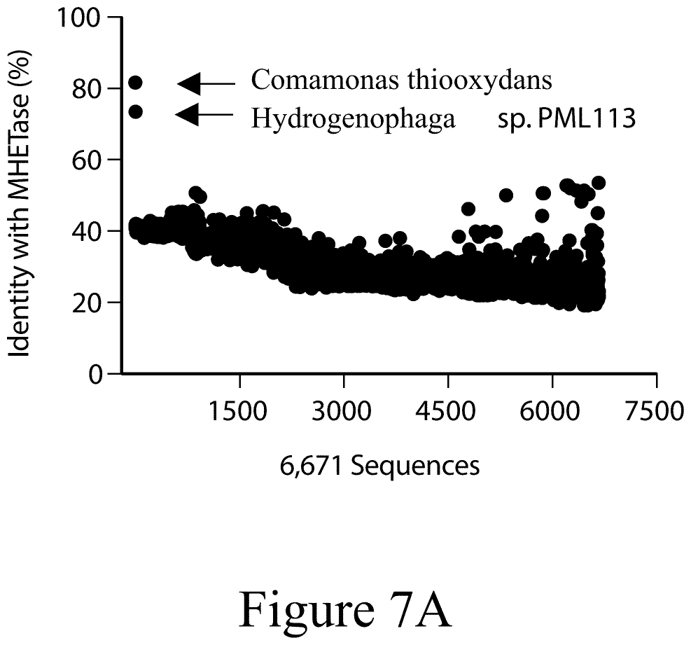

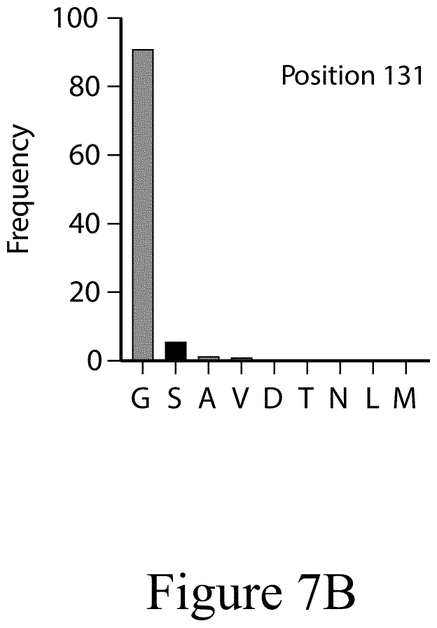

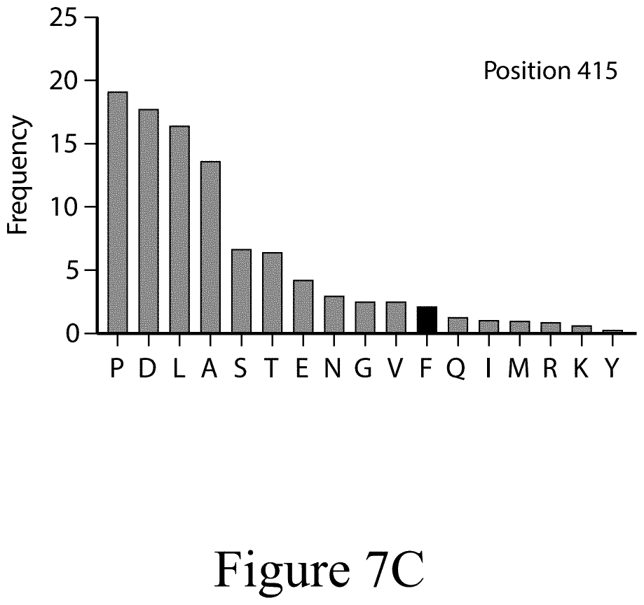

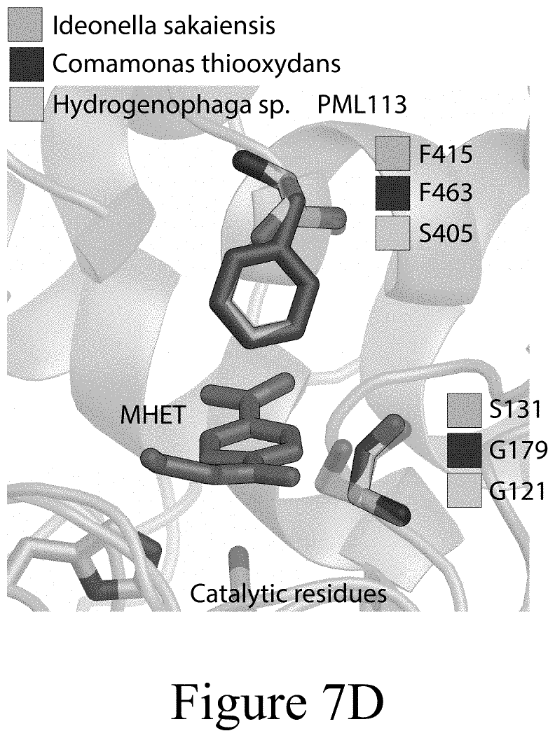

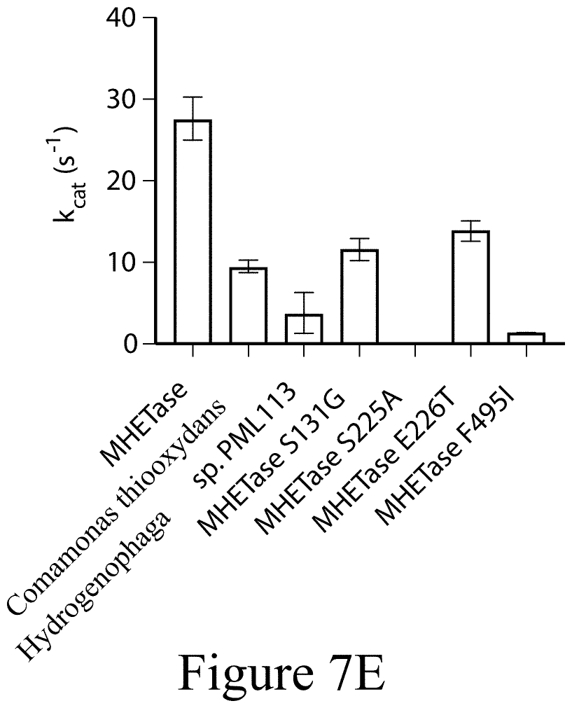

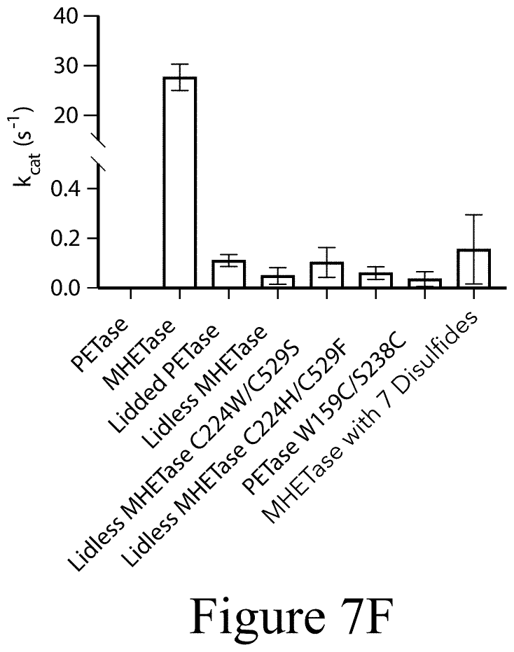

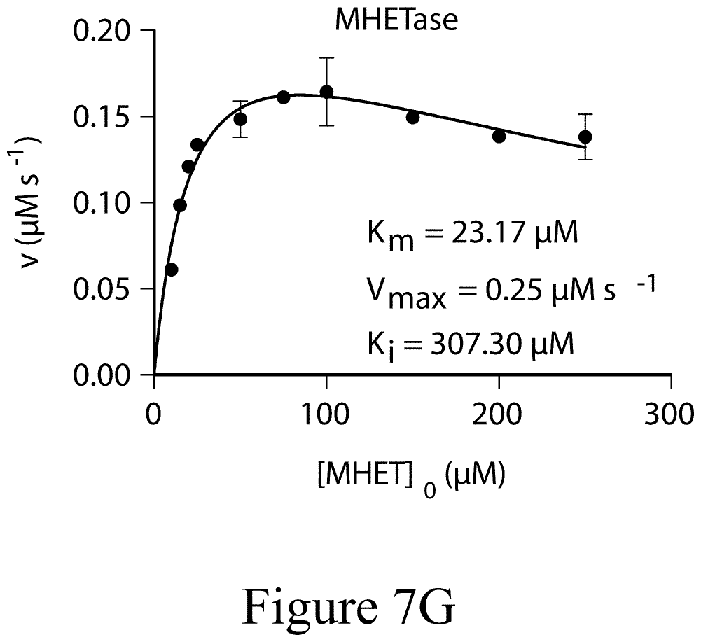

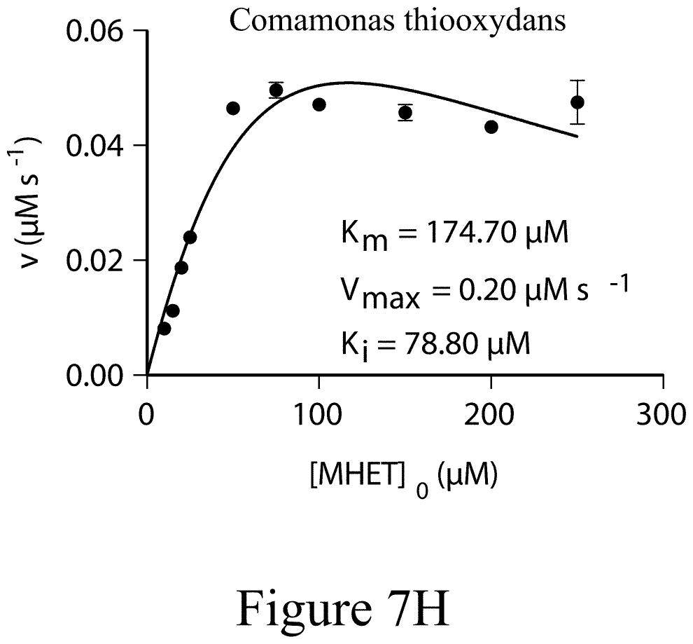

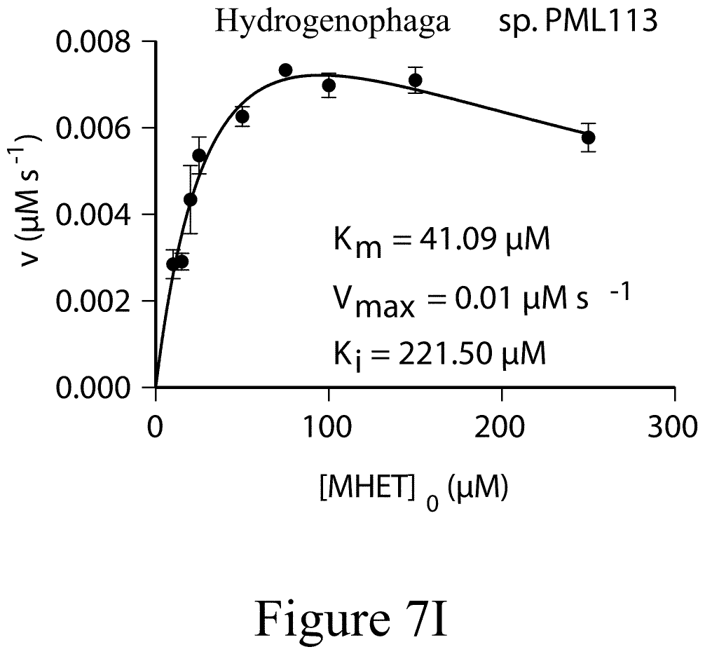

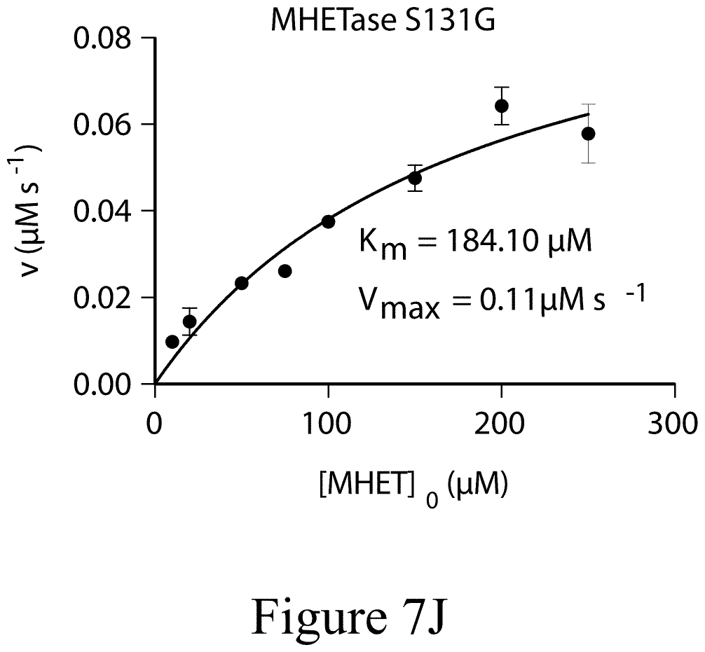

Further, using the multiple sequence alignment of 6,671 tannase family sequences, conservation analysis was performed with MHETase sequence positions as a reference (see FIGS. 4 and 5A through 5D), which shows that most positions in the active site are highly conserved. Notable exceptions are positions 257, 411, 415, and 416, which exhibit low conservation scores and are less conserved than 80% of MHETase positions overall (see Panels B and C of FIG. 4). It is noteworthy that position 131 is a well-conserved glycine in 91% o of tannase family sequences but serine appears at position 131 in MHETase. Furthermore, the ten cysteine positions in MHETase that form five disulfide bonds are highly conserved in the tannase family (see FIGS. 6A and 6B). Although a sixth disulfide bond exists in AoFaeB, less than 8% of tannase family sequences exhibit this sixth disulfide bond, and the sixth 68.91+/−8.66 Mutation of this lipase box residue to threonine (E226T) yielded a ˜50% reduction in MHET activity relative to the wild-type MHETase. Mutation of the catalytic serine (S225A), as expected, produced an inactive enzyme. FIGS. 7A through 7J illustrated other aspects according to some embodiments of the present disclosure.

In an embodiment, a MHETase-8 amino acid linker-PETase chimeric enzyme was created having a DNA sequence of SEQ ID NO: 35 and an expressed polypeptide sequence of SEQ ID NO: 36. The expressed chimeric enzyme with an 8 aa linker (SEQ ID NO: 36) exhibited a turnover number of 68.91+/−8.66⁻¹. In an embodiment, a MHETase-12 amino acid linker-PETase chimeric enzyme was created having a DNA sequence of SEQ ID NO: 37 and an expressed polypeptide sequence of SEQ ID NO: 38. The expressed chimeric enzyme with a 12 aa linker (SEQ ID NO: 38) exhibited a turnover number of 76.94+/−12.89 s⁻¹. In an embodiment, a MHETase-20 amino acid linker-PETase chimeric enzyme was created having a DNA sequence of SEQ ID NO: 39 and an expressed polypeptide sequence of SEQ ID NO: 40. The expressed chimeric enzyme with a 20 amino acid linker (SEQ ID NO: 40) exhibited a turnover number of 56.25+/−4.27 s⁻¹.

### Methods:

Plasmid construction (see Table 2 for plasmid construction, Table 3 for synthesized DNA fragments and (where applicable) translated polypeptide sequences, and Table 3 for primers): pET-21b(+) (EMD Millipore)-based plasmids for expression of the various Ideonella sakaiensis PETase and MHETase enzymes, as well as homologous, and mutant proteins were either synthesized by Twist Bioscience or constructed using NEBuilder® HiFi DNA Assembly Master Mix (New England Biolabs) and/or the Q5® Site-Directed Mutagenesis Kit (New England Biolabs) such that each protein has a C-terminal hexa-histidine epitope tag. For DNA assembly, DNA fragments were either amplified using Q5® High-Fidelity 2X Master Mix (New England Biolabs) or synthesized by Integrated DNA Technologies. Kits and master mixes were used according to the manufacturer's instructions. Plasmids were initially transformed into NEB® 5-alpha F′Iq Competent E. coli (New England Biolabs) and confirmed using Sanger sequencing by GENEWIZ, Inc.

Protein expression and purification: For initial screening for soluble protein expression of the proteins of interest, various cell lines and induction methods were used to purify protein for kinetic assays. For expression and purification, OverExpress™ E. coli C41 (DE3) (Lucigen) cells were transformed with pET21b(+) plasmid constructed with the gene of interest. Single colonies from transformation were then inoculated into a starter culture of Luria Broth (LB) media containing 100 μg/mL ampicillin and grown at 37° C. overnight. The starter culture was inoculated at a 100-fold dilution into a 2xYT medium containing 100 μg/mL ampicillin and grown at 37° C. until the optical density measured at 600 nM (OD600) reached between 0.6 and 0.8. Protein expression was then induced by addition of isopropyl β-D-1-thiogalactopyranoside (IPTG) to a final concentration of 1 mM. Cells were maintained at 20° C. for 18 to 24 hours following IPTG induction, harvested by centrifugation, and stored at −80° C. until purification. Harvested cells were resuspended in a lysis buffer (300 mM NaCl, 10 mM imidazole, 20 mM Tris HCl, pH 8.0) and lysed using a bead beater (BioSpec Products, Inc.). Lysate was clarified by centrifugation at 40,000×g for 45 minutes. Clarified lysate was then applied to a 5 mL HisTrap HP (GE Healthcare) Ni-NTA column using an ÄKTA Pure chromatography system (GE Healthcare) and eluted using 300 mM NaCl, 300 mM imidazole, 20 mM Tris HCl, pH 8.0. Resulting fractions containing proteins of interest were applied to a Sephacryl S-100 26/60 HR (GE Healthcare) size exclusion column equilibrated with 100 mM NaCl, 20 mM Tris HCl, pH 7.5 for biochemical assays, or the fractions were applied to a Superdex 75 pg 16/60 (GE Healthcare) size exclusion column equilibrated with 100 mM NaCl, 20 mM Tris HCl, pH 7.5 for crystallography. Protein in eluted fractions from Ni-NTA and size exclusion columns were assessed using SDS-PAGE with Coomassie staining and Western blot using primary antibody against the hexa-histidine epitope tag (Invitrogen). Total protein was assessed by BCA assay. For proteins that did not express, or expressed in inclusion bodies, using the above-described expression protocol, additional E. coli expression cell lines were tested, including Rosetta 2 (DE3) (Novagen), BL21 (DE3), and Lemo21 (DE3) (New England Biolabs), as was expression by autoinduction at 30° C. in ZYP-5052 media.

MHETase:PETase fusion proteins: Fusion proteins were expressed and purified as described above with the following noted exceptions: Single colonies from transformation into C41 (DE3) competent cells were used to inoculate a starter culture of 200 mL Terrific Broth (TB) media containing 100 μg/mL ampicillin for overnight outgrowth at 37° C. From the starter culture, 50 mL was used to inoculate 1 L of TB media containing 100 μg/mL ampicillin. For purification, cells were disrupted by sonication. In the final chromatography step a Superdex 200 pg 16/600 (GE Healthcare) size exclusion column equilibrated with 100 mM NaCl, 20 mM Tris HCl, pH 7.5 was used.

Crystallography. After purification, as described above, MHETase protein was concentrated to a range of concentrations (9-14 mg/mL) and dialyzed into 100 mM NaCl, 10 mM Tris, pH 7.0 for crystallography.

For seleno-methionine labeling of MHETase, K-MOPS minimal media was used. Cells were grown to an OD600 of 0.5 after which 100 mg/L of DL-seleno-methionine (Sigma), 100 mg/L lysine, threonine and phenylalanine, leucine, isoleucine and valine were added as solids. IPTG (1 mM final concentration) was then added after 20 min and cells were grown for a further 16 h at 20° C. Seleno-methionine labeled protein was purified as described above. MHETase was crystallized at a range of concentrations from 9-14 mg/mL by sitting-drop vapor diffusion. Several conditions yielded crystals, four of which were used to obtain datasets, one of which contained seleno-methionine labelled protein. The crystals were cryo-cooled in liquid nitrogen after the addition of glycerol to 20% (v/v) while leaving the other components of the mother liquor at the same concentration. Seleno-methionine MHETase crystals belonging to space group P22121 were used to obtain phase information using the 103 beamline at the Diamond Light Source (Oxford, UK). Data were obtained from 3600 images collected at 0.9795 A with 0.1° increments. All images were integrated using XDS (4) and scaled using SCALA. Phases were obtained using PHASERSAD in the CCP4i software in combination with PARROT and SHELXD. The initial output was subsequently built using BUCCANEER and further refined using iterative rounds of COOT and PHENIX. One molecule of MHETase was observed in the asymmetric unit of the P22121 seleno-methionine SAD dataset. Three additional native datasets, each containing 1800 images collected at 0.1° increments, were collected at beamline 103 of the Diamond Light Source. The structure of native MHETase were obtained using molecular replacement from a refined molecule of MHETase obtained initially from the seleno-methionine SAD data. All structures were refined using iterative rounds of COOT and PHENIX.

Determination of enzyme turnover rates. Comparative assays for each enzyme were performed at the same enzyme and substrate concentration. Reactions were performed in triplicate over a 15 min time course using 5 nM enzyme concentration and 250 μM MHET in 90 mM NaCl, 10% (v/v) DMSO, 45 mM sodium phosphate, pH 7.5, at 30° C. Reactions were terminated using an equal volume of 100% methanol followed by heat treatment at 85° C. for 10 min. Product and substrate were quantified by HPLC. Apparent turnover rate (kcat) was determined by terephthalic acid (TPA) produced.

Michaelis-Menten kinetics of MHETase and variants. Reactions were performed in triplicate over a 10 min time course using 5 nM enzyme and substrate concentrations ranging from 10 μM to 250 μM MHET in 90 mM NaCl, 10% (v/v) DMSO, 45 mM sodium phosphate, pH 7.5, at 30° C. Each reaction was terminated using an equal volume of 100% methanol and heat treatment at 85° C. for 10 min. Product and substrate were quantified by HPLC. Initial reaction velocities were calculated from TPA produced over time and kinetic parameters were determined by nonlinear regression of the initial velocities fit to the Michealis-Menten equation with substrate inhibition using GraphPad Prism version 8.4.1 for MacOS (GraphPad Software, San Diego, Calif. USA), as follows:

$$v = \frac{{V}_{max} [ S ]}{{K}_{m} + [ S ] ( 1 + \frac{[ S ]}{{K}_{i}} )} \qquad ( Eq . 1 )$$

While both substrate inhibition and product inhibition are possible in these reactions, the relationship between initial reaction velocity and initial substrate concentration indicates substrate inhibition predominates in these reaction conditions. Low substrate concentrations were considered in these kinetic studies in order to minimize the effect of substrate inhibition.

Enzymatic degradation of PET film. Amorphous PET film (2-3% crystallinity, Goodfellow, UK) was incubated with enzyme of interest in polypropylene tubes containing 90 mM NaCl, 10% (v/v) DMSO, 45 mM sodium phosphate, pH 7.5, at 30° C. for 96 hours. The reaction was terminated by addition of equal volume 100% methanol and PET coupons were removed from the reaction solution. The reaction solution was heat treated at 85° C. for 10 minutes. PET coupons were washed with consecutive rinses of 1% SDS, 100% DMSO, ultrapure water, and 95% ethanol. Coupons were then vacuum dried for 24 h at 60° C. for scanning electron microscopy.

Activity assay of MHETase with non-MHET substrates. Evaluation of MHETase activity was performed in triplicate using 5 nM enzyme concentration and 25 μM, 50 μM, and 250 μM substrate concentration at 30° C. for 24 h in a 0.5 mL reaction volume. The reaction was carried out in 90 mM NaCl, 10% (v/v) DMSO, 45 mM sodium phosphate, pH 7.5, reaction buffer with three concentrations of each substrate (MHET, MHEI, or MHEF). Reactions commenced upon addition of enzyme or an equal volume of reaction buffer for the no enzyme controls. At the end of 24 h the reactions were terminated using an equal volume of 100% DMSO and heat treatment at 85° C. for 10 min. Product and substrate were analyzed by HPLC. Values reported as percentage of substrate hydrolyzed into product.

HPLC method. Standards of BHET, TPA, 2,5-furandicarboxylic acid, and isophthalate were obtained from Sigma Aldrich. MHET, MHEI, and MHEF were synthesized as described above. Analyte analysis of samples was performed on an Agilent 1260 LC system (Agilent Technologies, Santa Clara, Calif.) equipped with a G1315A diode array detector (DAD). Each sample and standard were injected using a volume of 10 μL onto a Phenomenex Luna C18(2) column, 5 μm, 4.6×150 mm (Phenomenex, Torrance, Calif.). The column temperature was maintained at 40° C. and the mobile phase used to separate the analytes of interest was composed of 20 mM phosphoric acid in water (A) and 100% methanol (B). The separation was carried out using a constant flow rate of 0.6 mL/min and a gradient program of: at t=0 min (A)=80% and (B)=20%; at t=15 min (A)=35% and (B)=65%; at t=15.01 min through 20 min (A) =80% and (B)=20% for a total run time of 20 min. The calibration curve for each analyte was evaluated between concentrations of 0.1-200 mg/L. DAD detection at a wavelength of 240 nm was performed for each analyte. Ten calibration standards were used with an r2 coefficient of 0.995 or better and a calibration verification standard (CVS) at 100 mg/L for each analyte was analyzed every 18 samples to ensure the integrity of the initial calibration. Samples were diluted with an equal volume of ultrapure water for analysis.

TABLE 2
Plasmid Construction.

| Protein | Plasmid | Plasmid description | Construction details, reference, and other notes |
|---|---|---|---|
| PETase | pCJ135 | pET-21b(+) based plasmid for expression | Described previously in Austin, H. P., Allen, M. D., Donohoe, B. S., |
| | | of PETase from Ideonella sakaiensis 201- | Rorrer, N. A., Kearns, F. L., Silveira, R. L., Pollard, B. C., Dominick, |
| | | F6 (Genbank GAP38373.1), codon | G., Duman, R., Omari, El, K., Mykhaylyk, V., Wagner, A., Michener, |
| | | optimized for expression in E. coli K12, | W. E., Amore, A., Skaf, M. S., Crowley, M. F., Thorne, A. W., |
| | | with C-terminal His tag. Deposited to | Johnson, C. W., Woodcock, H. L., McGeehan, J. E., Beckham, G. T., |
| | | addgene as pET-21b(+)-Is-PETase | 2018. Characterization and engineering of a plastic-degrading |
| | | (Plasmid 112202). | aromatic polyesterase. Proc. Natl. Acad. Sci. U.S.A. 39, |
| | | | 201718804-8. |
| MHETase | pCJ136 | pET-21b(+) based plasmid for expression | pCJ136 was constructed by assembling the DNA fragment |
| | | of MHETase from Ideonella sakaiensis | CJ_MHETase_opt_Ec (synthesized by IDT), which omitted the |
| | | 201-F6 (Genbank GAP38911.1), codon | stop codon to enable a C-terminal His tag, into pET-21b(+) |
| | | optimized for expression in E. coli K12, | digested with Ndel and Xhol. |
| | | with C-terminal His tag. | |
| Lidded PETase | pCJ208 | pET-21b(+) based plasmid for expression | pCJ208 was constructed by assembling the DNA fragment |
| | | of PETase from Ideonella sakaiensis 201- | CJ_MHETLid (synthesized by IDT), which omitted the stop codon |
| | | F6 (Genbank GAP38373.1) incorporating | to enable a C-terminal His tag, into pCJ135 digested with with |
| | | the MHETase lid, codon optimized for | Ncol and Agel. |
| | | expression in E. coli K12, with C-terminal | |
| | | His tag. | |
| Lidless MHETase | pCJ209 | pET-21b(+) based plasmid for expression | pCJ209 was constructed by site-directed mutagenesis of pCJ136 |
| | | of MHETase from Ideonella sakaiensis | using NEB's Q5 ® Site-Directed Mutagenesis Kit according to the |
| | | 201-F6 (Genbank GAP38911.1) with the | manufacturer's instructions. pCJ136 was amplified using primer |
| | | lid removed, codon optimized for | pair oCJ787/oCJ788, incorporating the lid replacement from |
| | | expression in E. coli K12, with C-terminal | PETase. The resulting PCR product was treated with NEB's |
| | | His tag. | Kinase, Ligase, and Dpnl (KLD) enzyme mix. |
| MHETase | pCJ205 | pET-21b(+) based plasmid for expression | pCJ205 was constructed by site-directed mutagenesis of pCJ136 |
| C224A/C529A | | of MHETase from Ideonella sakaiensis | using NEB's Q5 ® Site-Directed Mutagenesis Kit according to the |
| | | 201-F6 (Genbank GAP38911.1), codon | manufacturer's instructions. pCJ136 was amplified using primer |
| | | optimized for expression in E. coli K12, | pair oCJ756/oCJ757 to generate a Cys224Ala mutation in the |
| | | with C-terminal His tag, incorporating | MHETase. The resulting PCR product was treated with NEB's |
| | | C224A and C529A mutations. | Kinase, Ligase, and Dpnl (KLD) enzyme mix. This plasmid was |
| | | | used as template for amplification with primer pair |
| | | | oCJ758/oCJ759 to generate a Cys529Ala mutation in the |
| | | | MHETase gene and the resulting PCR product was treated with |
| | | | NEB's Kinase, Ligase, and Dpnl (KLD) enzyme mix. |
| MHETase | pCJ201 | pET-21b(+) based plasmid for expression | pCJ201 was constructed by site-directed mutagenesis of pCJ136 |
| C224W/C529S | | of MHETase from Ideonella sakaiensis | using NEB's Q5 ® Site-Directed Mutagenesis Kit according to the |
| | | 201-F6 (Genbank GAP38911.1), codon | manufacturer's instructions. pCJ136 was amplified using primer |
| | | optimized for expression in E. coli K12, | pair oCJ756/oCJ760 to generate a Cys224Trp mutation in the |
| | | with C-terminal His tag, incorporating | MHETase gene. The resulting PCR product was treated with |
| | | C224W and C529SS mutations. | NEB's Kinase, Ligase, and Dpnl (KLD) enzyme mix. This plasmid |
| | | | was used as template for amplification with primer pair |
| | | | oCJ758/oCJ761 to generate a Cys529Ser mutation in the |
| | | | MHETase gene. The resulting PCR product was treated with |
| | | | NEB's Kinase, Ligase, and Dpnl (KLD) enzyme mix. |
| MHETase | pCJ204 | pET-21b(+) based plasmid for expression | pCJ204 was constructed by site-directed mutagenesis of pCJ136 |
| C224H/C529F | | of MHETase from Ideonella sakaiensis | using NEB's Q5 ® Site-Directed Mutagenesis Kit according to the |
| | | 201-F6 (Genbank GAP38911.1), codon | manufacturer's instructions. pCJ136 was amplified using primer |
| | | optimized for expression in E. coli K12, | pair oCJ756/oCJ762 to generate a Cys224His mutation in the |
| | | with C-terminal His tag, incorporating | MHETase gene. The resulting PCR product was treated with |
| | | C224H and C529F mutations. | NEB's Kinase, Ligase, and Dpnl (KLD) enzyme mix. This plasmid |
| | | | was used as template for amplification with primer pair |
| | | | oCJ758/oCJ763 to generate a Cys529Phe mutation in the |
| | | | MHETase gene. The resulting PCR product was treated with |
| | | | NEB's Kinase, Ligase, and Dpnl (KLD) enzyme mix. |
| PETase | pCJ202 | pET-21b(+) based plasmid for expression | pCJ202 was constructed by site-directed mutagenesis of pCJ135 |
| W159C/S238C | | of PETase from Ideonella sakaiensis 201- | using NEB's Q5 ® Site-Directed Mutagenesis Kit according to the |
| | | F6 (Genbank GAP38373.1), codon | manufacturer's instructions. pCJ135 was amplified using primer |
| | | optimized for expression in E. coli K12, | pair oCJ764/oCJ765 to generate a Trp159Cys mutation in the |
| | | with C-terminal His tag, incorporating | PETase gene. The resulting PCR product was treated with NEB's |
| | | W159C and S238C mutations. | Kinase, Ligase, and Dpnl (KLD) enzyme mix. This plasmid was |
| | | | used as template for amplification with primer pair |
| | | | oCJ766/oCJ767 to generate a Ser238Cys mutation in the PETase |
| | | | gene. The resulting PCR product was treated with NEB's Kinase, |
| | | | Ligase, and Dpnl (KLD) enzyme mix. |
| MHETase S225A | pCJ196 | pET-21b(+) based plasmid for expression | pCJ196 was constructed by site-directed mutagenesis of pCJ136 |
| | | of MHETase from Ideonella sakaiensis | using NEB's Q5 ® Site-Directed Mutagenesis Kit according to the |
| | | 201-F6 (Genbank GAP38911.1), codon | manufacturer's instructions. pCJ136 was amplified using primer |
| | | optimized for expression in E. coli K12, | pair oCJ756/oCJ757 to generate a Ser225Ala mutation. The |
| | | with C-terminal His tag, incorporating | resulting PCR product was treated with NEB's Kinase, Ligase, and |
| | | catalytic mutation, S225A | Dpnl (KLD) enzyme mix. |
| Comamonas | pCJ199 | pET-21b(+) based plasmid for expression | pCJ199 was synthesized by Twist Bioscience. |
| thiooxydans | | of the putative MHETase from | |
| | | Comamonas thiooxydans (Genbank | |
| | | WP_080747404.1) with signal peptide | |
| | | and C-terminal His tag, codon optimized | |
| | | for expression in E. coli K12. | |
| Comamonas | pCJ203 | pET-21b(+) based plasmid for expression | pCJ203 was constructed by removing the signal peptide from |
| thiooxydans with | | of the putative MHETase from | pCJ199 using NEB's Q5 ® Site-Directed Mutagenesis Kit according |
| truncated signal | | Comomonas thiooxydans (Genbank | to the manufacturer's instructions. pCJ199 was amplified with |
| peptide (Δ75aa) | | WP_080747404.1) without signal | oCJ770/oCJ771 to exclude the 75-residue signal peptide. The |
| | | peptide, with C-terminal His tag, codon | resulting PCR product was treated with NEB's Kinase, Ligase, and |
| | | optimized for expression in E. coli K12. | Dpnl (KLD) enzyme mix. |
| Hydrogenophaga | pCJ207 | pET-21b(+) based plasmid for expression | pCJ207 was synthesized by Twist Bioscience. |
| sp. PML113 | | of the putative MHETase from | |
| | | Hydrogenophaga sp. PML113 (Genbank | |
| | | WP_083293388.1) with signal peptide | |
| | | and C-terminal His tag, codon optimized | |
| | | for expression in E. coli K12. | |
| Hydrogenophaga | pCJ211 | pET-21b(+) based plasmid for expression | pCJ211 was constructed by removing the signal peptide from |
| sp. PML113 with | | of the putative MHETase from | pCJ207 using NEB's Q5 ® Site-Directed Mutagenesis Kit according |
| truncated signal | | Hydrogenophaga sp. PML113 (Genbank | to the manufacturer's instructions. pCJ207 was amplified using |
| peptide ((Δ19aa) | | WP_083293388.1) without signal | primer pair oCJ771/oCJ772 to exclude the 19-residue signal |
| | | peptide, with C-terminal His tag, codon | peptide. The resulting PCR product was treated with NEB's |
| | | optimized for expression in E. coli K12. | Kinase, Ligase, and Dpnl (KLD) enzyme mix. |
| Lidless MHETase | pCJ220 | pET-21b(+) based plasmid for expression | pCJ220 was constructed by site-directed mutagenesis of pCJ201 |
| C224W/C529S | | of MHETase from Ideonella sakaiensis | using NEB's Q5 ® Site-Directed Mutagenesis Kit according to the |
| | | 201-F6 (Genbank GAP38911.1), codon | manufacturer's instructions. pCJ201 was amplified with primer |
| | | optimized for expression in E. coli K12, | pair oCJ788/oCJ787 on Jan. 24, 2019 to generate a lid replacement |
| | | with C-terminal His tag, incorporating lid | from PETase. The resulting PCR product was treated with NEB's |
| | | deletion and C224W and C529S | Kinase, Ligase, and Dpnl (KLD) enzyme mix. |
| | | mutations from PETase active site. | |
| Lidless MHETase | pCJ221 | pET-21b(+) based plasmid for expression | pCJ221 was constructed by site-directed mutagenesis of pCJ204 |
| C224H/C549F | | of MHETase from Ideonella sakaiensis | using NEB's Q5 ® Site-Directed Mutagenesis Kit according to the |
| | | 201-F6 (Genbank GAP38911.1), codon | manufacturer's instructions. pCJ204 was amplified with primer |
| | | optimized for expression in E. coli K12, | pair oCJ788/oCJ787 on Jan. 24, 2019 to generate a lid replacement |
| | | with C-terminal His tag, incorporating lid | from wtPETase. The resulting PCR product was treated with |
| | | deletion and C224H and C529F | NEB's Kinase, Ligase, and Dpnl (KLD) enzyme mix. |
| | | mutations from PETase active site. | |
| MHETase S131G | pCJ197 | pET-21b(+) based plasmid for expression | pCJ197 was constructed by site-directed mutagenesis of pCJ136 |
| | | of MHETase from Ideonella sakaiensis | using NEB's Q5 ® Site-Directed Mutagenesis Kit according to the |
| | | 201-F6 (Genbank GAP38911.1), codon | manufacturer's instructions. pCJ136 was amplified using primer |
| | | optimized for expression in E. coli K12, | pair oCJ773/oCJ774 to generate a Ser131Gly mutation in the |
| | | with C-terminal His tag, incorporating | MHETase gene. The resulting PCR product was treated with |
| | | S131G mutation. | NEB's Kinase, Ligase, and Dpnl (KLD) enzyme mix. |
| MHETase F495I | pCJ198 | pET-21b(+) based plasmid for expression | pCJ198 was constructed by site-directed mutagenesis of pCJ136 |
| | | of MHETase from Ideonella sakaiensis | using NEB's Q5 ® Site-Directed Mutagenesis Kit according to the |
| | | 201-F6 (Genbank GAP38911.1), codon | manufacturer's instructions. pCJ136 was amplified using primer |
| | | optimized for expression in E. coli K12, | pair oCJ775/oCJ776 to generate a Phe495Ile mutation in the |
| | | with C-terminal His tag, incorporating | MHETase gene. The resulting PCR product was treated with |
| | | F495I mutation. | NEB's Kinase, Ligase, and Dpnl (KLD) enzyme mix. |
| MHETase with 6^{th} | pCJ200 | pET-21b(+) based plasmid for expression | pCJ200 was synthesized by Twist Bioscience. |
| Disulfide as | | of MHETase from Ideonella sakaiensis | |
| AoFaeB | | 201-F6 (Genbank GAP38911.1), codon | |
| | | optimized for expression in E. coli K12, | |
| | | with C-terminal His tag, incorporating | |
| | | mutations to introduce a 6th disulfide | |
| | | bond as in AoFaeB-2. | |
| MHETase E226T | pCJ206 | pET-21b(+) based plasmid for expression | pCJ206 was constructed by site-directed mutagenesis of pCJ136 |
| | | of MHETase from Ideonella sakaiensis | using NEB's Q5 ® Site-Directed Mutagenesis Kit according to the |
| | | 201-F6 (Genbank GAP38911.1), codon | manufacturer's instructions. pCJ136 was amplified using primer |
| | | optimized for expression in E. coli K12, | pair oCJ777/oCJ778 to generate a Glu226Thr mutation in the |
| | | with C-terminal His tag, incorporating | MHETase gene. The resulting PCR product was treated with |
| | | the E226T mutation to the putative | NEB's Kinase, Ligase, and Dpnl (KLD) enzyme mix. |
| | | lipase box. | |
| MHETase | pCJ217 | pET-21b(+) based plasmid for expression | pCJ217 was constructed by site-directed mutagenesis of pCJ136 |
| G489C/S530C | | of MHETase from Ideonella sakaiensis | using NEB's Q5 ® Site-Directed Mutagenesis Kit according to the |
| | | 201-F6 (Genbank GAP38911.1), codon | manufacturer's i]nstructions. pCJ136 was amplified using primer |
| | | optimized for expression in E. coli K12, | pair oCJ779/oCJ780 to generate Gly489Cys mutation in the |
| | | with C-terminal His tag, incorporating | MHETase gene. The resulting PCR product was treated with |
| | | two point mutations, G489C and S530C, | NEB's Kinase, Ligase, and Dpnl (KLD) enzyme mix. This plasmid |
| | | to introduce a 6th disulfide bond (from | was used as template for amplification with primer pair |
| | | PETase). | oCJ781/oCJ782 to generate a Ser530Cys mutation in the |
| | | | MHETase gene. The resulting PCR product was treated with |
| | | | NEB's Kinase, Ligase, and Dpnl (KLD) enzyme mix. |
| MHETase with 6^{th} | pCJ210 | pET-21b(+) based plasmid for expression | pCJ210 was constructed by site-directed mutagenesis of pCJ200 |
| and 7^{th} Disulfide | | of MHETase from Ideonella sakaiensis | using NEB's Q5 ® Site-Directed Mutagenesis Kit according to the |
| as AoFaeB | | 201-F6 (Genbank GAP38911.1), codon | manufacturer's instructions. pCJ200 was amplified using primer |
| | | optimized for expression in E. coli K12, | pair oCJ779/oCJ780 to generate Gly489Cys mutation in the |
| | | with C-terminal His tag, incorporating | MHETase gene. The resulting PCR product was treated with |
| | | mutations to introduce a 6th and 7th | NEB's Kinase, Ligase, and Dpnl (KLD) enzyme mix. This plasmid |
| | | disulfide bond as in AoFaeB-2. | was used as template for amplification with primer pair |
| | | | oCJ781/oCJ782 to generate a Ser530Cys mutation in the |
| | | | MHETase gene. The resulting PCR product was treated with |
| | | | NEB's Kinase, Ligase, and Dpnl (KLD) enzyme mix. |

TABLE 3
Synthesized DNA Fragments.

| Fragment | Sequence (5′-3′) | Description |
|---|---|---|
| CJ_MHETase_ | ctttaagaaggagatataCATATGcagaccaccgtgacca | The MHETase from |
| opt_Ec | ccatgctcctcgcgtccgtagcattagcggcttgcgccgg | Ideonella sakaiensis |
| (SEQ ID NO: 1) | aggaggttccactcctctgcctctaccgcagcagcagccg | strain 201-F6 |
| | cctcagcaggaaccgccacctcctcctgttccgctagcca | (Genbank GAP38911.1) |
| | gtcgcgccgcgtgtgaggcgctcaaagatggtaatggcga | was codon optimized for |
| | catggtttggccgaatgccgccacggttgtagaggttgca | expression in E. coli |
| | gcctggcgtgatgcagcaccggccacggcatcagccgcag | K12 MG1655 (Highly |
| | ccctgccggagcattgcgaagtatcaggcgcgattgccaa | Expressed Genes) |
| | gcgtactgggattgatgggtacccgtatgaaattaagttt | using the codon |
| | cgcctgcgcatgcccgctgagtggaacggccgttttttca | optimizer at |
| | tggagggtggcagtggtacgaacggctctctctcagcggc | http://genomes.urv.es/ |
| | gaccggaagtatcggcggcggtcagatcgcctcagcgctg | OPTIMIZER/ (guided random |
| | agtcgtaactttgcaacaattgctaccgacggaggacatg | method) (CAI: 0.658, |
| | acaatgcggtgaatgataatccggatgcgctcggtaccgt | ENc: 52, %GC: 58.5). |
| | cgcatttggtctcgatccccaggcacgcttagacatgggc | The stop codon was |
| | tacaactcctatgatcaggtgactcaggccggcaaagccg | omitted and overlaps |
| | ccgttgcacgcttttatggtcgcgcagccgacaagagcta | were added for assembly |
| | cttcatcggctgttcggagggcggccgcgagggcatgatg | into the expression |
| | ctgtcccagcgctttccatcacattacgatggcattgtgg | vector pET-21b(+) |
| | cgggcgcaccgggatatcagttgccgaaggccggaattag | digested with NdeI and |
| | tggcgcgtggaccacccagagcttagcgcccgccgccgtt | XhoI such that the |
| | ggcctggatgcccagggagtgccgctgattaataagagct | assembly would results |
| | tttctgacgcagacctccatttactgtcgcaggcgattct | in the addition of a |
| | cggaacatgcgacgccttggatggcctggccgacggcatc | C-terminal 6 His tag. |
| | gttgacaactaccgagcgtgccaagcggcttttgatccgg | The 6 His sequence in |
| | cgactgcagccaacccagcgaatggccaagccctgcagtg | pET-21b(+) is the same |
| | cgtgggcgcaaagacagccgattgcttatcgcccgtccaa | codon (CAC) repeated |
| | gttacggcgattaaacgagcgatggccggtccggtaaata | 6 times and so one of |
| | gcgcgggtacgccgttatataatagatgggcctgggacgc | the His codons in the |
| | aggtatgagcggtcttagtggtaccacttacaatcagggt | synthesized DNA |
| | tggcgcagctggtggctgggatcgtttaacagctcggcga | fragment was changed |
| | ataacgcacaacgtgtatctggtttctcagcgcggagctg | to CAT to enable |
| | gctggtggactttgctaccccgccggagccgatgcccatg | assembly. |
| | acccaagtcgccgcccgtatgatgaaatttgatttcgata | |
| | tcgatcctctgaaaatatgggctacttcgggccaatttac | |
| | ccagagtagtatggactggcacggtgccactagcaccgac | |
| | cttgctgcctttcgggaccgcggcggtaaaatgattctgt | |
| | atcacggaatgagcgatgccgcattctctgcactagatac | |
| | agcagattattatgaacgcctgggtgccgcaatgccgggc | |
| | gccgcgggctttgctcgtctgttcttggttccgggaatga | |
| | accattgctccgggggtccaggtaccgaccgctttgatat | |
| | gctaacaccgttagttgcatgggttgaacgtggggaagcc | |
| | cctgaccaaattagcgcctggagcggcacccccggctact | |
| | ttggtgtggccgcccgcactcgaccgttatgtccctatcc | |
| | gcagattgcgcgctataagggatcaggcgatatcaatacc | |
| | gaagcaaattttgcgtgtgccgctccaccgCTCGAGcacc | |
| | accatcaccaccactgagatccggct | |
| C_MHETase_Lid | tccgcgcttagctagccatggctttgtggttattaccatc | Lid region from |
| (SEQ ID NO: 2) | gatacgaacagcactctagaccagcccagcagccgtagct | the Ideonella |
| | cgcaacagatggccgcgcttcgtcaagttgcgagcttgaa | sakaiensis |
| | cgggaccagcagtagcccgatttacggaaaggtcgatact | strain |
| | gcccgcatgggtgtgatgggctggtcaatggggggcggcg | 201-F6 (Genbank |
| | gttcacttattagcgccgcgaacaacccgagtttaaaagc | GAP38911.1) MHETase, |
| | agcggcaccgcaggcgccaggatatcagttgccgaaggcc | with |
| | ggaattagtggcgcgtggaccacccagagcttagcgcccg | overlaps for assembly |
| | ccgccgttggcctggatgcccagggagtgccgctgattaa | into pCJ135. Assembly |
| | taagagcttttctgacgcagacctccatttactgtcgcag | will generate PETase |
| | gcgattctcggaacatgcgacgccttggatggcctggccg | with the MHETase lid. |
| | acggcatcgttgacaactaccgagcgtgccaagcggcttt | |
| | tgatccggcgactgcagccaacccagcgaatggccaagcc | |
| | ctgcagtgcgtgggcgcaaagacagccgattgcttatcgc | |
| | ccgtccaagttacggcgattaaacgagcgatggccggtcc | |
| | ggtaaatagcgcgggtacgccgttatataatagatgggcc | |
| | tgggacgcaggtatgagcggtcttagtggtaccacttaca | |
| | atcagggttggcgcagctggtggctgggatcgtttaacag | |
| | ctcggcgaataacgcacaacgtgtatctggtttctcagcg | |
| | cggagctggctggtggactttgctaccccgccggagccga | |
| | tgcccatgacccaagtcgccgcccgtatgatgaaatttga | |
| | tttcgatatcgatcctctgaaaatatgggctacttcgggc | |
| | caatttacccagagtagtatggactggcacggtgccacta | |
| | gcaccagcagtgttaccgtgccgacgctgattttcgcgtg | |
| | cgagaatgatagcattgcaccggtgaacagcagcgcgct | |
| pCJ199 | CATATGttcgtacgcaacgccgaccgtgccaagaattgta | pCJ199 was synthesized |
| (SEQ ID NO: 3) | tgcgcgcacctttacgcgtattcccactcaaggatacttt | by Twist Bioscience. |
| | tagcgcccagtgtgcgaatgtttcggtctggattaccagc | Only the sequence |
| | agcgtaccaccgctccgcgagcgtcacatggatcgccgcg | integrated between the |
| | tgacgcgccgcgatttaatgcaaactcgcatcttattaat | NdeI and XhoI sites |
| | gctcattgcagccactggcgtggcagcgtgtggcggagac | in pET-21b(+) is shown. |
| | ggtggttccacacctgccgcgcaaaatccccctttgcccc | |
| | tggccagtcgtgcggcttgcgaagcttttcaaggcaatag | |
| | caatagtatcgcgtggccccatcgcgcaaccgttgtggaa | |
| | gtggccacttggcacgaagcagagcctgcgaatgccacag | |
| | cagcggcgacgcccgagcactgtgagatttccggcgccat | |
| | tgctcgccgcaccggaattgatggatatccttacgagatt | |
| | aagtttcgcttacgtatgccctcagaatggaatggtcgct | |
| | tctttatggaaggggggggtggaaccaatgggtcattgag | |
| | tgccgctacagggtcccttggcggtggacaaactgcgtcg | |
| | gccttgagtcgtaattttgcaactattgcaaccgatggtg | |
| | gtcatgataatgctgtcaacaataatcctgatgcgctcgg | |
| | cactgtcgcttttggcatggaccctcaagcgcgcattgat | |
| | atgggatataattcctacgaccaggtgacccaagcgggaa | |
| | aggcggccgtagcgcaattttatggccgtgccccggataa | |
| | aagctattttattggctgtagcgaaggtgggcgcgagggg | |
| | atgatgttgtcccaacgctttccgagtcattatgacggaa | |
| | ttgtggcgggagcaccgggctaccaactcccaaaggcggg | |
| | catttctggcgcatggacgacgcaaagtctggcaccagca | |
| | gcggtgggcgtggatgctcaaaatgtacctttgatcaata | |
| | aggcgttttcggatgtcgatttacatcttctttcacgcgg | |
| | cattcttggtacttgcgatgccttggatggactcagcgat | |
| | ggaattgtgaacgacttccgtgcctgtcaagccgcctttg | |
| | accctgccactgcgttgaatcccgacaccagtcaaccctt | |
| | acaatgcactggtgctaagacgcctgattgcttaagtgcc | |
| | gcccaagtcactggcattaaacgcgccatgggtgggcctg | |
| | tggacagcgccggtgcggcattatataatcgctgggcatg | |
| | ggaccctggcatgtcggggctcaatggcacctcttacaat | |
| | cagggatggcgctcttggtggttagggagctacgcatcca | |
| | gcactaataatgcccaacgcgtcaatggcttttccgcccg | |
| | ctcttggttggttgattttgcgacgccgcctgaaccaatg | |
| | ccagtcacacaggttgctgctcgcatgatgaacttcaact | |
| | ttgacaccgacccgcctaagattcgcgcgactagtggccc | |
| | ctttactccatcgtctatggagtggcatggtgcaacgagc | |
| | actaatcttgcggccttccgcgaccgcggtgggaagctga | |
| | tgctctatcatggcatgtcagatgccgcgttctccgcatt | |
| | agacaccgcggattactacgagcgtttaggtgccgcgatg | |
| | ccgggcgccgccggcttcgcacgtcttttcttagttccag | |
| | gtatgaatcattgtagtggtggacccggcactgatcgttt | |
| | tgatatgcttactccccttgtggcctgggtggaacgtgat | |
| | aaggcgccagatcaagttagcgcttgggcaggcacaccgg | |
| | gctatttcggcgcaaccgcccgtacacgccccctttgtcc | |
| | atacccccaaatcgcacgttataagggctctggtgatatc | |
| | aatgccgaggcatcgtttgtgtgtgtggccccaCTCGAG | |
| pCJ200 | CATATGcagaccaccgtgaccaccatgctcctcgcgtccg | pCJ200 was synthesized |
| (SEQ ID NO: 4) | tagcattagcggcttgcgccggaggaggttccactcctct | by Twist Bioscience. |
| | gcctctaccgcagcagcagccgcctcagcaggaaccgcca | Only the sequence |
| | cctcctcctgttccgctagccagtcgcgccgcgtgtgagg | integrated between the |
| | cgctcaaagatggtaatggcgacatggtttggccgaatgc | NdeI and XhoI sites |
| | cgccacggttgtagaggttgcagcctacgtgccggcaggc | in pET-21b(+) is shown. |
| | gttaacatcagcatggcggataacccgagcatctgtggtg | |
| | gcgacgaggacccgattacttccaccttcgcgttctgcga | |
| | agtatcaggcgcgattgccaagcgtactgggattgatggg | |
| | tacccgtatgaaattaagtttcgcctgcgcatgcccgctg | |
| | agtggaacggccgttttttcatggagggtggcagtggtac | |
| | gaacggctgcctctcagcggcgaccggaagtatcggcggc | |
| | ggtcagatcgcctcagcgctgagtcgtaactttgcaacaa | |
| | ttgctaccgacggaggacatgacaatgcggtgaatgataa | |
| | tccggatgcgctcggtaccgtcgcatttggtctcgatccc | |
| | caggcacgcttagacatgggctacaactcctatgatcagg | |
| | tgactcaggccggcaaagccgccgttgcacgcttttatgg | |
| | tcgcgcagccgacaagagctacttcatcggctgttcggag | |
| | ggcggccgcgagggcatgatgctgtcccagcgctttccat | |
| | cacattacgatggcattgtggcgggcgcaccgggatatca | |
| | gttgccgaaggccggaattagtggcgcgtggaccacccag | |
| | agcttagcgcccgccgccgttggcctggatgcccagggag | |
| | tgccgctgattaataagagcttttctgacgcagacctcca | |
| | tttactgtcgcaggcgattctcggaacatgcgacgccttg | |
| | gatggcctggccgacggcatcgttgacaactaccgagcgt | |
| | gccaagcggcttttgatccggcgactgcagccaacccagc | |
| | gaatggccaagccctgcagtgcgtgggcgcaaagacagcc | |
| | gattgcttatcgcccgtccaagttacggcgattaaacgag | |
| | cgatggccggtccggtaaatagcgcgggtacgccgttata | |
| | taatagatgggcctgggacgcaggtatgagcggtcttagt | |
| | ggtaccacttacaatcagggttggcgcagctggtggctgg | |
| | gatcgtttaacagctcggcgaataacgcacaacgtgtatc | |
| | tggtttctcagcgcggagctggctggtggactttgctacc | |
| | ccgccggagccgatgcccatgacccaagtcgccgcccgta | |
| | tgatgaaatttgatttcgatatcgatcctctgaaaatatg | |
| | ggctacttcgggccaatttacccagagtagtatggactgg | |
| | cacggtgccactagcaccgaccttgctgcctttcgggacc | |
| | gcggcggtaaaatgattctgtatcacggaatgagcgatgc | |
| | cgcattctctgcactagatacagcagattattatgaacgc | |
| | ctgggtgccgcaatgccgggcgccgcgggctttgctcgtc | |
| | tgttcttggttccgggaatgaaccattgctccgggggtcc | |
| | aggtaccgaccgctttgatatgctaacaccgttagttgca | |
| | tgggttgaacgtggggaagcccctgaccaaattagcgcct | |
| | ggagcggcacccccggctactttggtgtggccgcccgcac | |
| | tcgaccgttatgtccctatccgcagattgcgcgctataag | |
| | ggatcaggcgatatcaataccgaagcaaattttgcgtgtg | |
| | ccgctccaccgCTCGAG | |
| pCJ207 | CATATGaagtctagcattccgattagcgtaggtatgctgg | pCJ207 was synthesized |
| (SEQ ID NO: 5) | ctaccgctctgatttccggttgcggtagcgttccggataa | by Twist Bioscience. |
| | caccagcaatgaaccgacggttccgctggcatccaaagaa | Only the sequence |
| | ttgtgcgagggcatcgcgtctggtgcgaccaaagtaaact | integrated between |
| | ggccgaaccagaacaccgtcgtaaaagcttcagtttggca | the NdeI and XhoI |
| | cgctgttaccccggcaaccgccaacgccccggaactgccg | sites in pET-21b(+) |
| | gaacattgcgaggtcactggctctatcaaccagcgtactg | is shown. |
| | gcgtggacggctatccgtatgaaatcaaaatgcgtctgcg | |
| | catgccggcagattggaacggccgtttcttcatggaaggc | |
| | ggtggaggtactaacgggagcctgtctgccgctctgggtt | |
| | cgctgggcggtggtcagaccagcaatgctctgagccgtcg | |
| | tttcgctaccgtttctaccgatggtggtcatgataacgca | |
| | gtgaacaacaatccggcggcgctgggttcggtcgctttcg | |
| | gcatggacccgcaggctcgcctggatcatggttacaactc | |
| | atacgatcaggttaccctggcgggcaagtcagcagtaagc | |
| | actttttacgggcgcggcccggacaaatcatacttcatcg | |
| | gctgttccgaaggcggtcgtgagggcatgatgttcagcca | |
| | gcgtttcccggcgcactatgacggcatcgtcgccggtgca | |
| | ccgggctaccagctgccgaaagcaggcatcagcggtgcat | |
| | ggacaacgcaatctctggcaccagcggccgttggtgttga | |
| | cccggacggtgcaccgctggtgaacaaatccttcagcgat | |
| | ccggacctgtatctgctgactcaggcaatcctgggcagct | |
| | gcgacgctctggacggtctggctgacggtatcgtcggcaa | |
| | ttattccgcgtgtcagtcgctgtttgacccgtctaccgcc | |
| | ttgaaccctgcgacgggacaaccactgcgttgcacgggcg | |
| | ctaagaccgacgactgcctaagcccggttcaggtggatgc | |
| | gatcaaacgtgctatgtccggtccagttgatactgccgga | |
| | accgccctgtataacaaatggccgtgggataccggtatgt | |
| | cgggcctgaacggcaccacttatttccagggctggcgcag | |
| | ctggtggctgggctcctacgacagctctactaacaacgcg | |
| | cagcgtgttaacggcagcagcgcacgctcttggctggtag | |
| | atttcgctactccgccggaacctgtaccgctgaaccaggt | |
| | ggccactcgtatgatgaactttgattttgatgttgacccg | |
| | ccgaaaatctttgctacctctggtctctttacccagccgt | |
| | ccatgcaatggcacggtgccacctcaaccgatctgaacgc | |
| | ttttcgctctcgcggtggcaagctgatgctgtaccacggc | |
| | atggctgacgcggcattcagcgcactggataccattgctt | |
| | attatgagcgcctgagcaccgcaatgccttccgtgtccga | |
| | cttttctcgcctgtttctggtgcctggtatggggcactgt | |
| | tccggcggtccgggcaccgatcgctttgatatgctgactc | |
| | cgctggtggcgtgggttgagaacggtactgcaccggctcg | |
| | cgtcgaagcgtcgtcctccactccgggttacttcggtgtt | |
| | tcggcccgcagccgccccctgtgcccgcatccgcagattg | |
| | cacgttataccgggtccggcgacattaacgaagccaccaa | |
| | ctttgtatgcggtaacccgCTCGAG | |
| MP8 DNA | atgcagaccaccgtgaccaccatgctcctcgcgtccgtag | DNA sequence for |
| (SEQ ID NO: 35) | cattagcggcttgcgccggaggaggttccactcctctgcc | MHETase-8 aa linker |
| | tctaccgcagcagcagccgcctcagcaggaaccgccacct | PETase |
| | cctcctgttccgctagccagtcgcgccgcgtgtgaggcgc | |
| | tcaaagatggtaatggcgacatggtttggccgaatgccgc | |
| | cacggttgtagaggttgcagcctggcgtgatgcagcaccg | |
| | gccacggcatcagccgcagccctgccggagcattgcgaag | |
| | tatcaggcgcgattgccaagcgtactgggattgatgggta | |
| | cccgtatgaaattaagtttcgcctgcgcatgcccgctgag | |
| | tggaacggccgttttttcatggagggtggcagtggtacga | |
| | acggctctctctcagcggcgaccggaagtatcggcggcgg | |
| | tcagatcgcctcagcgctgagtcgtaactttgcaacaatt | |
| | gctaccgacggaggacatgacaatgcggtgaatgataatc | |
| | cggatgcgctcggtaccgtcgcatttggtctcgatcccca | |
| | ggcacgcttagacatgggctacaactcctatgatcaggtg | |
| | actcaggccggcaaagccgccgttgcacgcttttatggtc | |
| | gcgcagccgacaagagctacttcatcggctgttcggaggg | |
| | cggccgcgagggcatgatgctgtcccagcgctttccatca | |
| | cattacgatggcattgtggcgggcgcaccgggatatcagt | |
| | tgccgaaggccggaattagtggcgcgtggaccacccagag | |
| | cttagcgcccgccgccgttggcctggatgcccagggagtg | |
| | ccgctgattaataagagcttttctgacgcagacctccatt | |
| | tactgtcgcaggcgattctcggaacatgcgacgccttgga | |
| | tggcctggccgacggcatcgttgacaactaccgagcgtgc | |
| | caagcggcttttgatccggcgactgcagccaacccagcga | |
| | atggccaagccctgcagtgcgtgggcgcaaagacagccga | |
| | ttgcttatcgcccgtccaagttacggcgattaaacgagcg | |
| | atggccggtccggtaaatagcgcgggtacgccgttatata | |
| | atagatgggcctgggacgcaggtatgagcggtcttagtgg | |
| | taccacttacaatcagggttggcgcagctggtggctggga | |
| | tcgtttaacagctcggcgaataacgcacaacgtgtatctg | |
| | gtttctcagcgcggagctggctggtggactttgctacccc | |
| | gccggagccgatgcccatgacccaagtcgccgcccgtatg | |
| | atgaaatttgatttcgatatcgatcctctgaaaatatggg | |
| | ctacttcgggccaatttacccagagtagtatggactggca | |
| | cggtgccactagcaccgaccttgctgcctttcgggaccgc | |
| | ggcggtaaaatgattctgtatcacggaatgagcgatgccg | |
| | cattctctgcactagatacagcagattattatgaacgcct | |
| | gggtgccgcaatgccgggcgccgcgggctttgctcgtctg | |
| | ttcttggttccgggaatgaaccattgctccgggggtccag | |
| | gtaccgaccgctttgatatgctaacaccgttagttgcatg | |
| | ggttgaacgtggggaagcccctgaccaaattagcgcctgg | |
| | agcggcacccccggctactttggtgtggccgcccgcactc | |
| | gaccgttatgtccctatccgcagattgcgcgctataaggg | |
| | atcaggcgatatcaataccgaagcaaattttgcgtgtgcc | |
| | gctccaccgggtggtggttctggtggttctggtcagacca | |
| | atccgtatgcgcgcggccccaaccctaccgccgcctcgtt | |
| | ggaagccagcgcgggaccctttaccgttcgtagctttacc | |
| | gttagccgtccgtccggatatggtgcagggaccgtctatt | |
| | acccaaccaatgcaggcggcaccgttggcgcgattgcaat | |
| | cgtccccgggtacaccgcgcgtcaaagcagcattaagtgg | |
| | tggggtccgcgcttagctagccatggctttgtggttatta | |
| | ccatcgatacgaacagcactctagaccagcccagcagccg | |
| | tagctcgcaacagatggccgcgcttcgtcaagttgcgagc | |
| | ttgaacgggaccagcagtagcccgatttacggaaaggtcg | |
| | atactgcccgcatgggtgtgatgggctggtcaatgggggg | |
| | cggcggttcacttattagcgccgcgaacaacccgagttta | |
| | aaagcagcggcaccgcaggcgccatgggactcttcaacca | |
| | acttcagcagtgttaccgtgccgacgctgattttcgcgtg | |
| | cgagaatgatagcattgcaccggtgaacagcagcgcgctg | |
| | ccgatttatgatagcatgtcccgcaacgcaaaacagtttc | |
| | tggaaattaacggcggtagccactcttgtgccaactctgg | |
| | gaacagcaaccaggcactgatcggaaaaaaaggggttgca | |
| | tggatgaaacgattcatggataatgacacccgttactcaa | |
| | ccttcgcctgtgagaatcccaacagcacacgcgtgtcgga | |
| | ttttcgcaccgcgaactgttccctcgagcaccaccatcac | |
| | caccactga | |
| MP8 aa | MQTTVTTMLLASVALAACAGGGSTPLPLPQQQPPQQEPPP | Amino acid sequence |
| (SEQ ID NO: 36) | PPVPLASRAACEALKDGNGDMVWPNAATVVEVAAWRDAAP | for MHETase- |
| | ATASAAALPEHCEVSGAIAKRTGIDGYPYEIKFRRMPAEW | 8 aa linker-PETase |
| | NGRFFMEGGSGTNGSLSAATGSIGGGQIASALSRNFATIA | |
| | TDGGHDNAVNDNPDALGTVAFGLDPQARLDMGYNSYDQVT | |
| | QAGKAAVARFYGRAADKSYFIGCSEGGREGMMLSQRFPSH | |
| | YDGIVAGAPGYQLPKAGISGAWTTQSLAPAAVGLDAQGVP | |
| | LINKSFSDADLHLLSQAILGTCDALDGLADGIVDNYRACQ | |
| | AAFDPATAANPANGQALQCVGAKTADCLSPVQVTAIKRAM | |
| | AGPVNSAGTPLYNRWAWDAGMSGLSGTTYNQGWRSWWLGS | |
| | FNSSANNAQRVSGFSARSWLVDFATPPEPMPMTQVAARMM | |
| | KFDFDIDPLKIWATSGQFTQSSMDWHGATSTDLAAFRDRG | |
| | GKMILYHGMSDAAFSALDTADYYERLGAAMPGAAGFARLF | |
| | LVPGMNHCSGGPGTDRFDMLTPLVAWVERGEAPDQISAWS | |
| | GTPGYFGVAARTRPLCPYPQJARYKGSGDINTEANFACAA | |
| | PPGGGSGGSGQTNPYARGPNPTAASLEASAGPFTVRSFTV | |
| | SRPSGYGAGTVYYPTNAGGTVGAIAIVPGYTARQSSIKWW | |
| | GPRLASHGFVVITIDTNSTLDQPSSRSSQQMAALRaVASL | |
| | NGTSSSPIYGKVDTARMGVMGWSMGGGGSLISAANNPSLK | |
| | AAAPQAPWDSSTNFSSVTVPTLIFACENDSIAPVNSSALP | |
| | IYDSMSRNAKQFLEINGGSHSCANSGNSNQALIGKKGVAW | |
| | MKRFMDNDTRYSTFACENPNSTRVSDFRTANCSLEHHHHH | |
| | H | |
| MP12 DNA | atgcagaccaccgtgaccaccatgctcctcgcgtccgtag | DNA sequence |
| (SEQ ID NO: 37) | cattagcggcttgcgccggaggaggttccactcctctgcc | for MHETase- |
| | tctaccgcagcagcagccgcctcagcaggaaccgccacct | 12 aa linker- |
| | cctcctgttccgctagccagtcgcgccgcgtgtgaggcgc | PETase |
| | tcaaagatggtaatggcgacatggtttggccgaatgccgc | |
| | cacggttgtagaggttgcagcctggcgtgatgcagcaccg | |
| | gccacggcatcagccgcagccctgccggagcattgcgaag | |
| | tatcaggcgcgattgccaagcgtactgggattgatgggta | |
| | cccgtatgaaattaagtttcgcctgcgcatgcccgctgag | |
| | tggaacggccgttttttcatggagggtggcagtggtacga | |
| | acggctctctctcagcggcgaccggaagtatcggcggcgg | |
| | tcagatcgcctcagcgctgagtcgtaactttgcaacaatt | |
| | gctaccgacggaggacatgacaatgcggtgaatgataatc | |
| | cggatgcgctcggtaccgtcgcatttggtctcgatcccca | |
| | ggcacgcttagacatgggctacaactcctatgatcaggtg | |
| | actcaggccggcaaagccgccgttgcacgcttttatggtc | |
| | gcgcagccgacaagagctacttcatcggctgttcggaggg | |
| | cggccgcgagggcatgatgctgtcccagcgctttccatca | |
| | cattacgatggcattgtggcgggcgcaccgggatatcagt | |
| | tgccgaaggccggaattagtggcgcgtggaccacccagag | |
| | cttagcgcccgccgccgttggcctggatgcccagggagtg | |
| | ccgctgattaataagagcttttctgacgcagacctccatt | |
| | tactgtcgcaggcgattctcggaacatgcgacgccttgga | |
| | tggcctggccgacggcatcgttgacaactaccgagcgtgc | |
| | caagcggcttttgatccggcgactgcagccaacccagcga | |
| | atggccaagccctgcagtgcgtgggcgcaaagacagccga | |
| | ttgcttatcgcccgtccaagttacggcgattaaacgagcg | |
| | atggccggtccggtaaatagcgcgggtacgccgttatata | |
| | atagatgggcctgggacgcaggtatgagcggtcttagtgg | |
| | taccacttacaatcagggttggcgcagctggtggctggga | |
| | tcgtttaacagctcggcgaataacgcacaacgtgtatctg | |
| | gtttctcagcgcggagctggctggtggactttgctacccc | |
| | gccggagccgatgcccatgacccaagtcgccgcccgtatg | |
| | atgaaatttgatttcgatatcgatcctctgaaaatatggg | |
| | ctacttcgggccaatttacccagagtagtatggactggca | |
| | cggtgccactagcaccgaccttgctgcctttcgggaccgc | |
| | ggcggtaaaatgattctgtatcacggaatgagcgatgccg | |
| | cattctctgcactagatacagcagattattatgaacgcct | |
| | gggtgccgcaatgccgggcgccgcgggctttgctcgtctg | |
| | ttcttggttccgggaatgaaccattgctccgggggtccag | |
| | gtaccgaccgctttgatatgctaacaccgttagttgcatg | |
| | ggttgaacgtggggaagcccctgaccaaattagcgcctgg | |
| | agcggcacccccggctactttggtgtggccgcccgcactc | |
| | gaccgttatgtccctatccgcagattgcgcgctataaggg | |
| | atcaggcgatatcaataccgaagcaaattttgcgtgtgcc | |
| | gctccaccgggtggtggttctggtggttctggtggtggtt | |
| | ctggtcagaccaatccgtatgcgcgcggccccaaccctac | |
| | cgccgcctcgttggaagccagcgcgggaccctttaccgtt | |
| | cgtagctttaccgttagccgtccgtccggatatggtgcag | |
| | ggaccgtctattacccaaccaatgcaggcggcaccgttgg | |
| | cgcgattgcaatcgtccccgggtacaccgcgcgtcaaagc | |
| | agcattaagtggtggggtccgcgcttagctagccatggct | |
| | ttgtggttattaccatcgatacgaacagcactctagacca | |
| | gcccagcagccgtagctcgcaacagatggccgcgcttcgt | |
| | caagttgcgagcttgaacgggaccagcagtagcccgattt | |
| | acggaaaggtcgatactgcccgcatgggtgtgatgggctg | |
| | gtcaatggggggcggcggttcacttattagcgccgcgaac | |
| | aacccgagtttaaaagcagcggcaccgcaggcgccatggg | |
| | actcttcaaccaacttcagcagtgttaccgtgccgacgct | |
| | gattttcgcgtgcgagaatgatagcattgcaccggtgaac | |
| | agcagcgcgctgccgatttatgatagcatgtcccgcaacg | |
| | caaaacagtttctggaaattaacggcggtagccactcttg | |
| | tgccaactctgggaacagcaaccaggcactgatcggaaaa | |
| | aaaggggttgcatggatgaaacgattcatggataatgaca | |
| | cccgttactcaaccttcgcctgtgagaatcccaacagcac | |
| | acgcgtgtcggattttcgcaccgcgaactgttccctcgag | |
| | caccaccatcaccaccactga | |
| MP12 aa | MQTTVTTMLLASVALAACAGGGSTPLPLPQQQPPQQEPPP | Amino Acid sequence |
| (SEQ ID NO: 38) | PPVPLASRAACEALKDGNGDMVWPNAATVVEVAAWRDAAP | for MHETase-12 aa |
| | ATASAAALPEHCEVSGAIAKRTGIDGYPYEIKFRLRMPAE | linker-PETase |
| | WNGRFFMEGGSGTNGSLSAATGSIGGGQIASALSRNFATI | |
| | ATDGGHDNAVNDNPDALGTVAFGLDPQARLDMGYNSYDQV | |
| | TQAGKAAVARFYGRAADKSYFIGCSEGGREGMMLSQRFPS | |
| | HYDGIVAGAPGYQLPKAGISGAWTTQSLAPAAVGLDAQGV | |
| | PLINKSFSDADLHLLSQAILGTCDALDGLADGIVDNYRAC | |
| | QAAFDPATAANPANGQALQCVGAKTADCLSPVQVTAIKRA | |
| | MAGPVNSAGTPLYNRWAWDAGMSGLSGTTYNQGWRSWWLG | |
| | SFNSSANNAQRVSGFSARSWLVDFATPPEPMPMTQVAARM | |
| | MKFDFDIDPLKIWATSGQFTQSSMDWHGATSTDLAAFRDR | |
| | GGKMILYHGMSDAAFSALDTADYYERLGAAMPGAAGFARL | |
| | FLVPGMNHCSGGPGTDRFDMLTPLVAWVERGEAPDQISAW | |
| | SGTPGYFGVAARTRPLCPYPQIARYKGSGDINTEANFACA | |
| | APPGGGSGGSGGGSGQTNPYARGPNPTAASLEASAGPFTV | |
| | RSFTVSRPSGYGAGTVYYPTNAGGTVGAIAIVPGYTARQS | |
| | SIKWWGPRLASHGFWITIDTNSTLDQPSSRSSQQMAALRQ | |
| | VASLNGTSSSPIYGKVDTARMGVMGWSMGGGGSLISAANN | |
| | PSLKAAAPQAPWDSSTNFSSVTVPTLIFACENDSIAPVNS | |
| | SALPIYDSMSRNAKQFLEINGGSHSCANSGNSNQALIGKK | |
| | GVAWMKRFMDNDTRYSTFACENPNSTRVSDFRTANCSLEH | |
| | HHHHH | |
| MP20 DNA | atgcagaccaccgtgaccaccatgctcctcgcgtccgtag | DNA sequence |
| (SEQ ID NO: 39) | cattagcggcttgcgccggaggaggttccactcctctgcc | for MHETase- |
| | tctaccgcagcagcagccgcctcagcaggaaccgccacct | 20 aa linker- |
| | cctcctgttccgctagccagtcgcgccgcgtgtgaggcgc | PETase |
| | tcaaagatggtaatggcgacatggtttggccgaatgccgc | |
| | cacggttgtagaggttgcagcctggcgtgatgcagcaccg | |
| | gccacggcatcagccgcagccctgccggagcattgcgaag | |
| | tatcaggcgcgattgccaagcgtactgggattgatgggta | |
| | cccgtatgaaattaagtttcgcctgcgcatgcccgctgag | |
| | tggaacggccgttttttcatggagggtggcagtggtacga | |
| | acggctctctctcagcggcgaccggaagtatcggcggcgg | |
| | tcagatcgcctcagcgctgagtcgtaactttgcaacaatt | |
| | gctaccgacggaggacatgacaatgcggtgaatgataatc | |
| | cggatgcgctcggtaccgtcgcatttggtctcgatcccca | |
| | ggcacgcttagacatgggctacaactcctatgatcaggtg | |
| | actcaggccggcaaagccgccgttgcacgcttttatggtc | |
| | gcgcagccgacaagagctacttcatcggctgttcggaggg | |
| | cggccgcgagggcatgatgctgtcccagcgctttccatca | |
| | cattacgatggcattgtggcgggcgcaccgggatatcagt | |
| | tgccgaaggccggaattagtggcgcgtggaccacccagag | |
| | cttagcgcccgccgccgttggcctggatgcccagggagtg | |
| | ccgctgattaataagagcttttctgacgcagacctccatt | |
| | tactgtcgcaggcgattctcggaacatgcgacgccttgga | |
| | tggcctggccgacggcatcgttgacaactaccgagcgtgc | |
| | caagcggcttttgatccggcgactgcagccaacccagcga | |
| | atggccaagccctgcagtgcgtgggcgcaaagacagccga | |
| | ttgcttatcgcccgtccaagttacggcgattaaacgagcg | |
| | atggccggtccggtaaatagcgcgggtacgccgttatata | |
| | atagatgggcctgggacgcaggtatgagcggtcttagtgg | |
| | taccacttacaatcagggttggcgcagctggtggctggga | |
| | tcgtttaacagctcggcgaataacgcacaacgtgtatctg | |
| | gtttctcagcgcggagctggctggtggactttgctacccc | |
| | gccggagccgatgcccatgacccaagtcgccgcccgtatg | |
| | atgaaatttgatttcgatatcgatcctctgaaaatatggg | |
| | ctacttcgggccaatttacccagagtagtatggactggca | |
| | cggtgccactagcaccgaccttgctgcctttcgggaccgc | |
| | ggcggtaaaatgattctgtatcacggaatgagcgatgccg | |
| | cattctctgcactagatacagcagattattatgaacgcct | |
| | gggtgccgcaatgccgggcgccgcgggctttgctcgtctg | |
| | ttcttggttccgggaatgaaccattgctccgggggtccag | |
| | gtaccgaccgctttgatatgctaacaccgttagttgcatg | |
| | ggttgaacgtggggaagcccctgaccaaattagcgcctgg | |
| | agcggcacccccggctactttggtgtggccgcccgcactc | |
| | gaccgttatgtccctatccgcagattgcgcgctataaggg | |
| | atcaggcgatatcaataccgaagcaaattttgcgtgtgcc | |
| | gctccaccgggtggtggttctggtggttctggtggtggtt | |
| | ctggtggtggtggttctggtggttctggtcagaccaatcc | |
| | gtatgcgcgcggccccaaccctaccgccgcctcgttggaa | |
| | gccagcgcgggaccctttaccgttcgtagctttaccgtta | |
| | gccgtccgtccggatatggtgcagggaccgtctattaccc | |
| | aaccaatgcaggcggcaccgttggcgcgattgcaatcgtc | |
| | cccgggtacaccgcgcgtcaaagcagcattaagtggtggg | |
| | gtccgcgcttagctagccatggctttgtggttattaccat | |
| | cgatacgaacagcactctagaccagcccagcagccgtagc | |
| | tcgcaacagatggccgcgcttcgtcaagttgcgagcttga | |
| | acgggaccagcagtagcccgatttacggaaaggtcgatac | |
| | tgcccgcatgggtgtgatgggctggtcaatggggggcggc | |
| | ggttcacttattagcgccgcgaacaacccgagtttaaaag | |
| | cagcggcaccgcaggcgccatgggactcttcaaccaactt | |
| | cagcagtgttaccgtgccgacgctgattttcgcgtgcgag | |
| | aatgatagcattgcaccggtgaacagcagcgcgctgccga | |
| | tttatgatagcatgtcccgcaacgcaaaacagtttctgga | |
| | aattaacggcggtagccactcttgtgccaactctgggaac | |
| | agcaaccaggcactgatcggaaaaaaaggggttgcatgga | |
| | tgaaacgattcatggataatgacacccgttactcaacctt | |
| | cgcctgtgagaatcccaacagcacacgcgtgtcggatttt | |
| | cgcaccgcgaactgttccctcgagcaccaccatcaccacc | |
| | actga | |
| MP20 aa | MQTTVTTMLLASVALAACAGGGSTPLPLPQQQPPQQEPPP | Amino acid sequence |
| (SEQ ID NO: 40) | PPVPLASRAACEALKDGNGDMVWPNAATVVEVAAWRDAAP | for MHETase-20 aa |
| | ATASAAALPEHCEVSGAIAKRTGIDGYPYEIKFRLRMPAE | linker-PETase |
| | WNGRFFMEGGSGTNGSLSAATGSIGGGQIASALSRNFATI | |
| | ATDGGHDNAVNDNPDALGTVAFGLDPQARLDMGYNSYDQV | |
| | TQAGKAAVARFYGRAADKSYFIGCSEGGREGMMLSQRFPS | |
| | HYDGIVAGAPGYQLPKAGISGAWTTQSLAPAAVGLDAQGV | |
| | PLINKSFSDADLHLLSQAILGTCDALDGLADGIVDNYRAC | |
| | QAAFDPATAANPANGQALQCVGAKTADCLSPVQVTAIKRA | |
| | MAGPVNSAGTPLYNRWAWDAGMSGLSGTTYNQGWRSWWLG | |
| | SFNSSANNAQRVSGFSARSWLVDFATPPEPMPMTQVAARM | |
| | MKFDFDIDPLKIWATSGQFTQSSMDWHGATSTDLAAFRDR | |
| | GGKMILYHGMSDAAFSALDTADYYERLGAAMPGAAGFARL | |
| | FLVPGMNHCSGGPGTDRFDMLTPLVAWVERGEAPDQISAW | |
| | SGTPGYFGVAARTRPLCPYPQIARYKGSGDINTEANFACA | |
| | APPGGGSGGSGGGSGGGGSGGSGQTNPYARGPNPTAASLE | |
| | ASAGPFTVRSFTVSRPSGYGAGTVYYPTNAGGTVGAIAIV | |
| | PGYTARQSSIKWWGPRLASHGFWITIDTNSTLDQPSSRSS | |
| | QQMAALRQVASLNGTSSSPIYGKVDTARMGVMGWSMGGGG | |
| | SLISAANNPSLKAAAPQAPWDSSTNFSSVTVPTLIFACEN | |
| | DSIAPVNSSALPIYDSMSRNAKQFLEINGGSHSCANSGNS | |
| | NQALIGKKGVAWMKRFMDNDTRYSTFACENPNSTRVSDFR | |
| | TANCSLEHHHHHH | |
| PM8 DNA | atgaacttcccccgtgcctcgcgccttatgcaggctgctg | DNA sequence for PETase- |
| (SEQ ID NO: 41) | tgctgggcggccttatggccgtttccgcagcggccaccgc | 8 aa linker- |
| | gcagaccaatccgtatgcgcgcggccccaaccctaccgcc | MHETase |
| | gcctcgttggaagccagcgcgggaccctttaccgttcgta | |
| | gctttaccgttagccgtccgtccggatatggtgcagggac | |
| | cgtctattacccaaccaatgcaggcggcaccgttggcgcg | |
| | attgcaatcgtccccgggtacaccgcgcgtcaaagcagca | |
| | ttaagtggtggggtccgcgcttagctagccatggctttgt | |
| | ggttattaccatcgatacgaacagcactctagaccagccc | |
| | agcagccgtagctcgcaacagatggccgcgcttcgtcaag | |
| | ttgcgagcttgaacgggaccagcagtagcccgatttacgg | |
| | aaaggtcgatactgcccgcatgggtgtgatgggctggtca | |
| | atggggggcggcggttcacttattagcgccgcgaacaacc | |
| | cgagtttaaaagcagcggcaccgcaggcgccatgggactc | |
| | ttcaaccaacttcagcagtgttaccgtgccgacgctgatt | |
| | ttcgcgtgcgagaatgatagcattgcaccggtgaacagca | |
| | gcgcgctgccgatttatgatagcatgtcccgcaacgcaaa | |
| | acagtttctggaaattaacggcggtagccactcttgtgcc | |
| | aactctgggaacagcaaccaggcactgatcggaaaaaaag | |
| | gggttgcatggatgaaacgattcatggataatgacacccg | |
| | ttactcaaccttcgcctgtgagaatcccaacagcacacgc | |
| | gtgtcggattttcgcaccgcgaactgttccggtggtggtt | |
| | ctggtggttctggttgcgccggaggaggttccactcctct | |
| | gcctctaccgcagcagcagccgcctcagcaggaaccgcca | |
| | cctcctcctgttccgctagccagtcgcgccgcgtgtgagg | |
| | cgctcaaagatggtaatggcgacatggtttggccgaatgc | |
| | cgccacggttgtagaggttgcagcctggcgtgatgcagca | |
| | ccggccacggcatcagccgcagccctgccggagcattgcg | |
| | aagtatcaggcgcgattgccaagcgtactgggattgatgg | |
| | gtacccgtatgaaattaagtttcgcctgcgcatgcccgct | |
| | gagtggaacggccgttttttcatggagggtggcagtggta | |
| | cgaacggctctctctcagcggcgaccggaagtatcggcgg | |
| | cggtcagatcgcctcagcgctgagtcgtaactttgcaaca | |
| | attgctaccgacggaggacatgacaatgcggtgaatgata | |
| | atccggatgcgctcggtaccgtcgcatttggtctcgatcc | |
| | ccaggcacgcttagacatgggctacaactcctatgatcag | |
| | gtgactcaggccggcaaagccgccgttgcacgcttttatg | |
| | gtcgcgcagccgacaagagctacttcatcggctgttcgga | |
| | gggcggccgcgagggcatgatgctgtcccagcgctttcca | |
| | tcacattacgatggcattgtggcgggcgcaccgggatatc | |
| | agttgccgaaggccggaattagtggcgcgtggaccaccca | |
| | gagcttagcgcccgccgccgttggcctggatgcccaggga | |
| | gtgccgctgattaataagagcttttctgacgcagacctcc | |
| | atttactgtcgcaggcgattctcggaacatgcgacgcctt | |
| | ggatggcctggccgacggcatcgttgacaactaccgagcg | |
| | tgccaagcggcttttgatccggcgactgcagccaacccag | |
| | cgaatggccaagccctgcagtgcgtgggcgcaaagacagc | |
| | cgattgcttatcgcccgtccaagttacggcgattaaacga | |
| | gcgatggccggtccggtaaatagcgcgggtacgccgttat | |
| | ataatagatgggcctgggacgcaggtatgagcggtcttag | |
| | tggtaccacttacaatcagggttggcgcagctggtggctg | |
| | ggatcgtttaacagctcggcgaataacgcacaacgtgtat | |
| | ctggtttctcagcgcggagctggctggtggactttgctac | |
| | cccgccggagccgatgcccatgacccaagtcgccgcccgt | |
| | atgatgaaatttgatttcgatatcgatcctctgaaaatat | |
| | gggctacttcgggccaatttacccagagtagtatggactg | |
| | gcacggtgccactagcaccgaccttgctgcctttcgggac | |
| | cgcggcggtaaaatgattctgtatcacggaatgagcgatg | |
| | ccgcattctctgcactagatacagcagattattatgaacg | |
| | cctgggtgccgcaatgccgggcgccgcgggctttgctcgt | |
| | ctgttcttggttccgggaatgaaccattgctccgggggtc | |
| | caggtaccgaccgctttgatatgctaacaccgttagttgc | |
| | atgggttgaacgtggggaagcccctgaccaaattagcgcc | |
| | tggagcggcacccccggctactttggtgtggccgcccgca | |
| | ctcgaccgttatgtccctatccgcagattgcgcgctataa | |
| | gggatcaggcgatatcaataccgaagcaaattttgcgtgt | |
| | gccgctccaccgctcgagcaccaccatcaccaccactga | |
| PM12 DNA | atgaacttcccccgtgcctcgcgccttatgcaggctgctg | DNA sequence for PETase- |
| (SEQ ID NO: 42) | tgctgggcggccttatggccgtttccgcagcggccaccgc | 12 aa linker- |
| | gcagaccaatccgtatgcgcgcggccccaaccctaccgcc | MHETase |
| | gcctcgttggaagccagcgcgggaccctttaccgttcgta | |
| | gctttaccgttagccgtccgtccggatatggtgcagggac | |
| | cgtctattacccaaccaatgcaggcggcaccgttggcgcg | |
| | attgcaatcgtccccgggtacaccgcgcgtcaaagcagca | |
| | ttaagtggtggggtccgcgcttagctagccatggctttgt | |
| | ggttattaccatcgatacgaacagcactctagaccagccc | |
| | agcagccgtagctcgcaacagatggccgcgcttcgtcaag | |
| | ttgcgagcttgaacgggaccagcagtagcccgatttacgg | |
| | aaaggtcgatactgcccgcatgggtgtgatgggctggtca | |
| | atggggggcggcggttcacttattagcgccgcgaacaacc | |
| | cgagtttaaaagcagcggcaccgcaggcgccatgggactc | |
| | ttcaaccaacttcagcagtgttaccgtgccgacgctgatt | |
| | ttcgcgtgcgagaatgatagcattgcaccggtgaacagca | |
| | gcgcgctgccgatttatgatagcatgtcccgcaacgcaaa | |
| | acagtttctggaaattaacggcggtagccactcttgtgcc | |
| | aactctgggaacagcaaccaggcactgatcggaaaaaaag | |
| | gggttgcatggatgaaacgattcatggataatgacacccg | |
| | ttactcaaccttcgcctgtgagaatcccaacagcacacgc | |
| | gtgtcggattttcgcaccgcgaactgttccggtggtggtt | |
| | ctggtggttctggtggtggttctggttgcgccggaggagg | |
| | ttccactcctctgcctctaccgcagcagcagccgcctcag | |
| | caggaaccgccacctcctcctgttccgctagccagtcgcg | |
| | ccgcgtgtgaggcgctcaaagatggtaatggcgacatggt | |
| | ttggccgaatgccgccacggttgtagaggttgcagcctgg | |
| | cgtgatgcagcaccggccacggcatcagccgcagccctgc | |
| | cggagcattgcgaagtatcaggcgcgattgccaagcgtac | |
| | tgggattgatgggtacccgtatgaaattaagtttcgcctg | |
| | cgcatgcccgctgagtggaacggccgttttttcatggagg | |
| | gtggcagtggtacgaacggctctctctcagcggcgaccgg | |
| | aagtatcggcggcggtcagatcgcctcagcgctgagtcgt | |
| | aactttgcaacaattgctaccgacggaggacatgacaatg | |
| | cggtgaatgataatccggatgcgctcggtaccgtcgcatt | |
| | tggtctcgatccccaggcacgcttagacatgggctacaac | |
| | tcctatgatcaggtgactcaggccggcaaagccgccgttg | |
| | cacgcttttatggtcgcgcagccgacaagagctacttcat | |
| | cggctgttcggagggcggccgcgagggcatgatgctgtcc | |
| | cagcgctttccatcacattacgatggcattgtggcgggcg | |
| | caccgggatatcagttgccgaaggccggaattagtggcgc | |
| | gtggaccacccagagcttagcgcccgccgccgttggcctg | |
| | gatgcccagggagtgccgctgattaataagagcttttctg | |
| | acgcagacctccatttactgtcgcaggcgattctcggaac | |
| | atgcgacgccttggatggcctggccgacggcatcgttgac | |
| | aactaccgagcgtgccaagcggcttttgatccggcgactg | |
| | cagccaacccagcgaatggccaagccctgcagtgcgtggg | |
| | cgcaaagacagccgattgcttatcgcccgtccaagttacg | |
| | gcgattaaacgagcgatggccggtccggtaaatagcgcgg | |
| | gtacgccgttatataatagatgggcctgggacgcaggtat | |
| | gagcggtcttagtggtaccacttacaatcagggttggcgc | |
| | agctggtggctgggatcgtttaacagctcggcgaataacg | |
| | cacaacgtgtatctggtttctcagcgcggagctggctggt | |
| | ggactttgctaccccgccggagccgatgcccatgacccaa | |
| | gtcgccgcccgtatgatgaaatttgatttcgatatcgatc | |
| | ctctgaaaatatgggctacttcgggccaatttacccagag | |
| | tagtatggactggcacggtgccactagcaccgaccttgct | |
| | gcctttcgggaccgcggcggtaaaatgattctgtatcacg | |
| | gaatgagcgatgccgcattctctgcactagatacagcaga | |
| | ttattatgaacgcctgggtgccgcaatgccgggcgccgcg | |
| | ggctttgctcgtctgttcttggttccgggaatgaaccatt | |
| | gctccgggggtccaggtaccgaccgctttgatatgctaac | |
| | accgttagttgcatgggttgaacgtggggaagcccctgac | |
| | caaattagcgcctggagcggcacccccggctactttggtg | |
| | tggccgcccgcactcgaccgttatgtccctatccgcagat | |
| | tgcgcgctataagggatcaggcgatatcaataccgaagca | |
| | aattttgcgtgtgccgctccaccgctcgagcaccaccatc | |
| | accaccactga | |
| PM20 DNA | atgaacttcccccgtgcctcgcgccttatgcaggctgctg | DNA sequence for PETase- |
| (SEQ ID NO: 43) | tgctgggcggccttatggccgtttccgcagcggccaccgc | 20 aa linker- |
| | gcagaccaatccgtatgcgcgcggccccaaccctaccgcc | MHETase |
| | gcctcgttggaagccagcgcgggaccctttaccgttcgta | |
| | gctttaccgttagccgtccgtccggatatggtgcagggac | |
| | cgtctattacccaaccaatgcaggcggcaccgttggcgcg | |
| | attgcaatcgtccccgggtacaccgcgcgtcaaagcagca | |
| | ttaagtggtggggtccgcgcttagctagccatggctttgt | |
| | ggttattaccatcgatacgaacagcactctagaccagccc | |
| | agcagccgtagctcgcaacagatggccgcgcttcgtcaag | |
| | ttgcgagcttgaacgggaccagcagtagcccgatttacgg | |
| | aaaggtcgatactgcccgcatgggtgtgatgggctggtca | |
| | atggggggcggcggttcacttattagcgccgcgaacaacc | |
| | cgagtttaaaagcagcggcaccgcaggcgccatgggactc | |
| | ttcaaccaacttcagcagtgttaccgtgccgacgctgatt | |
| | ttcgcgtgcgagaatgatagcattgcaccggtgaacagca | |
| | gcgcgctgccgatttatgatagcatgtcccgcaacgcaaa | |
| | acagtttctggaaattaacggcggtagccactcttgtgcc | |
| | aactctgggaacagcaaccaggcactgatcggaaaaaaag | |
| | gggttgcatggatgaaacgattcatggataatgacacccg | |
| | ttactcaaccttcgcctgtgagaatcccaacagcacacgc | |
| | gtgtcggattttcgcaccgcgaactgttccggtggtggtt | |
| | ctggtggttctggtggtggttctggtggtggtggttctgg | |
| | tggttctggttgcgccggaggaggttccactcctctgcct | |
| | ctaccgcagcagcagccgcctcagcaggaaccgccacctc | |
| | ctcctgttccgctagccagtcgcgccgcgtgtgaggcgct | |
| | caaagatggtaatggcgacatggtttggccgaatgccgcc | |
| | acggttgtagaggttgcagcctggcgtgatgcagcaccgg | |
| | ccacggcatcagccgcagccctgccggagcattgcgaagt | |
| | atcaggcgcgattgccaagcgtactgggattgatgggtac | |
| | ccgtatgaaattaagtttcgcctgcgcatgcccgctgagt | |
| | ggaacggccgttttttcatggagggtggcagtggtacgaa | |
| | cggctctctctcagcggcgaccggaagtatcggcggcggt | |
| | cagatcgcctcagcgctgagtcgtaactttgcaacaattg | |
| | ctaccgacggaggacatgacaatgcggtgaatgataatcc | |
| | ggatgcgctcggtaccgtcgcatttggtctcgatccccag | |
| | gcacgcttagacatgggctacaactcctatgatcaggtga | |
| | ctcaggccggcaaagccgccgttgcacgcttttatggtcg | |
| | cgcagccgacaagagctacttcatcggctgttcggagggc | |
| | ggccgcgagggcatgatgctgtcccagcgctttccatcac | |
| | attacgatggcattgtggcgggcgcaccgggatatcagtt | |
| | gccgaaggccggaattagtggcgcgtggaccacccagagc | |
| | ttagcgcccgccgccgttggcctggatgcccagggagtgc | |
| | cgctgattaataagagcttttctgacgcagacctccattt | |
| | actgtcgcaggcgattctcggaacatgcgacgccttggat | |
| | ggcctggccgacggcatcgttgacaactaccgagcgtgcc | |
| | aagcggcttttgatccggcgactgcagccaacccagcgaa | |
| | tggccaagccctgcagtgcgtgggcgcaaagacagccgat | |
| | tgcttatcgcccgtccaagttacggcgattaaacgagcga | |
| | tggccggtccggtaaatagcgcgggtacgccgttatataa | |
| | tagatgggcctgggacgcaggtatgagcggtcttagtggt | |
| | accacttacaatcagggttggcgcagctggtggctgggat | |
| | cgtttaacagctcggcgaataacgcacaacgtgtatctgg | |
| | tttctcagcgcggagctggctggtggactttgctaccccg | |
| | ccggagccgatgcccatgacccaagtcgccgcccgtatga | |
| | tgaaatttgatttcgatatcgatcctctgaaaatatgggc | |
| | tacttcgggccaatttacccagagtagtatggactggcac | |
| | ggtgccactagcaccgaccttgctgcctttcgggaccgcg | |
| | gcggtaaaatgattctgtatcacggaatgagcgatgccgc | |
| | attctctgcactagatacagcagattattatgaacgcctg | |
| | ggtgccgcaatgccgggcgccgcgggctttgctcgtctgt | |
| | tcttggttccgggaatgaaccattgctccgggggtccagg | |
| | taccgaccgctttgatatgctaacaccgttagttgcatgg | |
| | gttgaacgtggggaagcccctgaccaaattagcgcctgga | |
| | gcggcacccccggctactttggtgtggccgcccgcactcg | |
| | accgttatgtccctatccgcagattgcgcgctataaggga | |
| | tcaggcgatatcaataccgaagcaaattttgcgtgtgccg | |
| | ctccaccgctcgagcaccaccatcaccaccactga | |

TABLE 4
Primers.

| Oligo | Sequence (5′ -> 3′) | Description |
|---|---|---|
| oC756 | teggagggcggccg | For linear amplification of Ideonella sakaiensis |
| (SEQ ID | | MHETase expression plasmid, |
| NO: 6) | | pCJ136, at Ser225 F |
| oC757 | ggcgccgatgaagta | For linear amplification of Ideonella sakaiensis |
| (SEQ ID | gctcttgtcggc | MHETase expression plasmid, |
| NO: 7) | | pCJ136, at Ser225 R with Cys224Ala mutation |
| oC758 | tccgggggtccaggt | For linear amplification of Ideonella sakaiensis |
| (SEQ ID | acc | MHETase expression plasmid, |
| NO: 8) | | pCJ136, at Ser530 F |
| oCJ759 | ggcatggttcattcc | For linear amplification of Ideonella sakaiensis |
| (SEQ ID | cggaaccaagaacag | MHETase expression plasmid, |
| NO: 9) | | pCJ136, at Ser530 R with Cys529Ala mutation |
| oCJ760 | ccagccgatgaagta | For linear amplification of Ideonella sakaiensis |
| (SEQ ID | gctcttgtcggc | MHETase expression plasmid, |
| NO: 10) | | pCJ136, at Ser225 R with Cys224Trp mutation |
| oCJ761 | gctatggttcattcc | For linear amplification of Ideonella sakaiensis |
| (SEQ ID | cggaaccaagaacag | MHETase expression plasmid, |
| NO: 11) | | pCJ136, at Ser530 R with Cys529Ser mutation |
| oCJ762 | gtggccgatgaagta | For linear amplification of Ideonella sakaiensis |
| (SEQ ID | gctcttgtcggc | MHETase expression plasmid, |
| NO: 12) | | pCJ136, at Ser225 R with Cys224His mutation |
| oC763 | gaaatggttcattcc | For linear amplification of Ideonella sakaiensis |
| (SEQ ID | cggaaccaagaacag | MHETase expression plasmid, |
| NO: 13) | | pCJ136, at Ser530 R with Cys529Phe mutation |
| oC764 | tcaatggggggcggc | For linear amplification of Ideonella sakaiensis |
| (SEQ ID | g | PETase expression plasmid, |
| NO: 14) | | pCJ135, at Ser160 F |
| oC765 | gcagcccatcacacc | For linear amplification of Ideonella sakaiensis |
| (SEQ ID | catgcgg | PETase expression plasmid, |
| NO: 15) | | pCJ135, at Ser160 R with Trypl59Cys mutation |
| oC766 | tgtgccaactctggg | For linear amplification of Ideonella sakaiensis |
| (SEQ ID | aacagc | PETase expression plasmid, |
| NO: 16) | | pCJ135, at Cys239 F |
| oC767 | gcagtggctaccgcc | For linear amplification of Ideonella sakaiensis |
| (SEQ ID | gttaatttccag | PETase expression plasmid, |
| NO: 17) | | pCJ135, at Cys239 R with Ser238Cys mutation |
| oC768 | gagggcggccgcga | For linear amplification of Ideonella sakaiensis |
| (SEQ ID | | MHETase expression plasmid, |
| NO: 18) | | pCJ136, at Glu226 F |
| oCJ769 | ggcacagccgatgaa | For linear amplification of Ideonella sakaiensis |
| (SEQ ID | gtagctcttgtcg | MHETase expression plasmid, |
| NO: 19) | | pCJ136, at Glu226 R with Ser225Ala mutation |
| oC770 | tgtggcggagacggt | For linear amplification of Comamonas thiooxydans |
| (SEQ ID | g | expression plasmid, |
| NO: 20) | | pCJ199, at Cys76 F |
| oCJ771 | catatgtatatctcc | For linear amplification of putative Comomonas |
| (SEQ ID | ttctta | thiooxydans expression |
| NO: 21) | aagttaaacaaaatt | plasmid, pCJ199, and Hydrogenophaga sp. PML113 |
| | atttcta | expression plasmid, |
| | | pCJ211, at MetIR |
| oCJ772 | tgcggtagcgttccg | For linear amplification of Hydrogenophaga sp. |
| (SEQ ID | g | PML113 expression plasmid, |
| NO: 22) | | pCJ211, at Cys20 F |
| oC773 | ggcggtacgaacggc | For linear amplification of Ideonella sakaiensis |
| (SEQ ID | tctctctcag | MHETase expression plasmid, |
| NO: 23) | | pCJ136, at Ser131 F with Ser131Gly mutation |
| oCJ774 | gccaccctccatgaa | For linear amplification of Ideonella sakaiensis |
| (SEQ ID | aaaacgg | MHETase expression plasmid, |
| NO: 24) | | pCJ136, at Ser131 R |
| oC775 | atctctgcactagat | For linear amplification of Ideonella sakaiensis |
| (SEQ ID | acagcagattattat | MHETase expression plasmid, |
| NO: 25) | gaac | pCJ136, at Phe495 F with Phe495Ile mutation |
| oC776 | tgcggcatcgctcat | For linear amplification of Ideonella sakaiensis |
| (SEQ ID | tcc | MHETase expression plasmid, |
| NO: 26) | | pCJ136, at Phe495 R |
| oC777 | ggcggccgcgagg | For linear amplification of Ideonella sakaiensis |
| (SEQ ID | | MHETase expression plasmid, |
| NO: 27) | | pCJ136, atGly227 F |
| oC778 | ggtcgaacagccgat | For linear amplification of Ideonella sakaiensis |
| (SEQ ID | gaagtagctcttgtc | MHETase expression plasmid, |
| NO: 28) | | pCJ136, at Gly227 R with Glu226Thr mutation |
| oC779 | tgcatgagcgatgcc | For linear amplification of Ideonella sakaiensis |
| (SEQ ID | gcattctctg | MHETase expression plasmid, |
| NO: 29) | | pCJ136, at Gly489 F with Gly489Cys mutation |
| oC780 | gtgatacagaatcat | For linear amplification of Ideonella sakaiensis |
| (SEQ ID | tttaccgccgcg | MHETase expression plasmid, |
| NO: 30) | | pCJ136, atGly489 R |
| oC781 | gggggtccaggtacc | For linear amplification of Ideonella sakaiensis |
| (SEQ ID | gac | MHETase expression plasmid, |
| NO: 31) | | pCJ136, at Ser530 F |
| oC782 | gcagcaatggttcat | For linear amplification of Ideonella sakaiensis |
| (SEQ ID | tcccggaacc | MHETase expression plasmid, |
| NO: 32) | | pCJ136, at Ser530 R with Ser530Cys mutation |
| oC787 | caaccaacttcgacc | For linear amplification of Ideonella sakaiensis |
| (SEQ ID | ttgctgcctttcggg | MHETase expression plasmid, |
| NO: 33) | ac | pCJ136, F |
| oC788 | aagagtcccacggtg | For linear amplification of Ideonella sakaiensis |
| (SEQ ID | cgcccgcc | MHETase expression plasmid, |
| NO: 34) | | pCJ136, R |

Table 5 depicts the Michaelis-Menten kinetic parameters of fitting initial reaction velocities of enzymatic turnover for Is MHETase, Is MHETase S131G, Comamonas thiooxydans MHETase, and Hydrogenophaga sp. PML113 MHETase at MHET substrate concentrations between 10.M and 250.M using the Michaelis-Menten model with substrate inhibition. Non-linear regression was performed using GraphPad Prism (8.4.1) along with 95 confidence intervals for each parameter and R² value given for fit of the model to the data.

TABLE 5

| | Kₘ | Vₘₐₓ | Kᵢ | | k_{cat}/Kₘ |
|---|---|---|---|---|---|
| Enzyme | (μM) | (μM s⁻¹) | (μM) | R² | (μM⁻¹ s⁻¹) |
| Is MHETase | 23.17 ± 1.65 | 0.252 ± 0.045 | 307.3 ± 20.65 | 0.9027 | 2.17 |
| Is MHETase | 995.10 ± 19.58 | 0.455 ± 0.071 | 102.7 ± 6.05 | 0.9174 | 0.09 |
| S131G | | | | | |
| Comamonas | 174.70 ± 4.75 | 0.203 ± 0.047 | 78.8 ± 3.04 | 0.9328 | 0.23 |
| thiooxydans | | | | | |
| MHETase | | | | | |
| Hydrogenophaga | 41.09 ± 3.38 | 0.013 ± 0.003 | 221.5 ± 19.01 | 0.9269 | 0.13 |
| sp. PML113 | | | | | |
| MHETase | | | | | |

The foregoing discussion and examples have been presented for purposes of illustration and description. The foregoing is not intended to limit the aspects, embodiments, or configurations to the form or forms disclosed herein. In the foregoing Detailed Description for example, various features of the aspects, embodiments, or configurations are grouped together in one or more embodiments, configurations, or aspects for the purpose of streamlining the disclosure. The features of the aspects, embodiments, or configurations, may be combined in alternate aspects, embodiments, or configurations other than those discussed above. This method of disclosure is not to be interpreted as reflecting an intention that the aspects, embodiments, or configurations require more features than are expressly recited in each claim. Rather, as the following claims reflect, inventive aspects lie in less than all features of a single foregoing disclosed embodiment, configuration, or aspect. While certain aspects of conventional technology have been discussed to facilitate disclosure of some embodiments of the present invention, the Applicants in no way disclaim these technical aspects, and it is contemplated that the claimed invention may encompass one or more of the conventional technical aspects discussed herein. Thus, the following claims are hereby incorporated into this Detailed Description, with each claim standing on its own as a separate aspect, embodiment, or configuration.

What is claimed is:

1. A non-naturally occurring enzyme comprising:
a first polypeptide that catalyzes the hydrolysis of a polyester to produce mono-(2-hydroxyethyl) terephthalate (MHET);
a second polypeptide that catalyzes the cleavage of MHET to produce at least one of terephthalic acid or ethylene glycol; and
a third polypeptide that links the first polypeptide with the second polypeptide.

2. The enzyme of claim 1, wherein the enzyme has a sequence identity that is greater than 80% to SEQ ID NO: 36.

3. The enzyme of claim 2, having a turnover rate of up to 69⁻¹.

4. The enzyme of claim 2, wherein the third polypeptide is 8 amino acids.

5. The enzyme of claim 1, wherein the enzyme has a sequence identity that is greater than 80% to SEQ ID NO: 38.

6. The enzyme of claim 5, having a turnover rate of up to 77⁻¹.

7. The enzyme of claim 6, wherein the third polypeptide is 12 amino acids.

8. The enzyme of claim 1, wherein the enzyme has a sequence identity that is greater than 80% to SEQ ID NO: 40.

9. The enzyme of claim 8, having a turnover rate of up to 56⁻¹.

10. The enzyme of claim 9, wherein the third polypeptide is 20 amino acids.

11. The enzyme of claim 1, wherein the polyester comprises at least one of polyethylene terephthalate (PET), polyglycolic acid, polylactic acid, polycaprolactone, polyhydroxyalkanoate, polyhydroxybutyrate, polyethylene adipate, polybutylene succinate, poly(3-hydroxybutyrate-co-3-hydroxyvalerate), polybutylene terephthalate, polytrimethylene terephthalate, or polyethylene naphthlate.

12. The enzyme of claim 1, wherein the third polypeptide comprises between 1 and 100 amino acids.

13. The enzyme of claim 1, wherein the third polypeptide comprises at least one of glycine, serine, proline, or threonine.

14. The enzyme of claim 1, wherein the third polypeptide covalently links the C-terminus of the second polypeptide to the N-terminus of the first polypeptide.

15. The enzyme of claim 1, further comprising:
a fourth polypeptide capable of catalyzing hydrolysis of a polyester to produce mono-(2-hydroxyethyl) terephthalate (MHET); and
a fifth polypeptide, wherein:
the fifth polypeptide covalently links the fourth polypeptide with the second polypeptide.

16. The enzyme of claim 1, further comprising a mutation of at least one of a S to G, a T to L, F, or Y, a E to N, T, D, Q, or G, a R to F, E, T, A, Y, I, S, W, L, V, Q, G, M, or N, a F to P, D, L, A, S, T, E, N, G, or V, a S to A, G, Q, P, E, D, or V, a S to R, A, K, Q, or G, a T to V or L, or a F to I.

17. The enzyme of claim 16, wherein the mutation occurs in the second polypeptide.

18. A genetically modified organism that expresses the enzyme of claim 1.

19. The organism of claim 18, wherein the organism comprises at least one of Pseudomonas putida or Escherichia coli.

20. A method for degrading a polyester, the method comprising contacting the organism of claim 18 with the polyester.


SEQUENCE LISTING (43 sequences)

SEQ ID NO: 1 - DNA, 1866 bp, Artificial Sequence
  Codon optimized MHETase
ctttaagaaggagatatacatatgcagaccaccgtgaccaccatgctcctcgcgtccgtagcattagcggcttgcgccggaggaggttccactcctctgcctctaccgcagcagcagccgcctcagcaggaaccgccacctcctcctgttccgctagccagtcgcgccgcgtgtgaggcgctcaaagatggtaatggcgacatggtttggccgaatgccgccacggttgtagaggttgcagcctggcgtgatgcagcaccggccacggcatcagccgcagccctgccggagcattgcgaagtatcaggcgcgattgccaagcgtactgggattgatgggtacccgtatgaaattaagtttcgcctgcgcatgcccgctgagtggaacggccgttttttcatggagggtggcagtggtacgaacggctctctctcagcggcgaccggaagtatcggcggcggtcagatcgcctcagcgctgagtcgtaactttgcaacaattgctaccgacggaggacatgacaatgcggtgaatgataatccggatgcgctcggtaccgtcgcatttggtctcgatccccaggcacgcttagacatgggctacaactcctatgatcaggtgactcaggccggcaaagccgccgttgcacgcttttatggtcgcgcagccgacaagagctacttcatcggctgttcggagggcggccgcgagggcatgatgctgtcccagcgctttccatcacattacgatggcattgtggcgggcgcaccgggatatcagttgccgaaggccggaattagtggcgcgtggaccacccagagcttagcgcccgccgccgttggcctggatgcccagggagtgccgctgattaataagagcttttctgacgcagacctccatttactgtcgcaggcgattctcggaacatgcgacgccttggatggcctggccgacggcatcgttgacaactaccgagcgtgccaagcggcttttgatccggcgactgcagccaacccagcgaatggccaagccctgcagtgcgtgggcgcaaagacagccgattgcttatcgcccgtccaagttacggcgattaaacgagcgatggccggtccggtaaatagcgcgggtacgccgttatataatagatgggcctgggacgcaggtatgagcggtcttagtggtaccacttacaatcagggttggcgcagctggtggctgggatcgtttaacagctcggcgaataacgcacaacgtgtatctggtttctcagcgcggagctggctggtggactttgctaccccgccggagccgatgcccatgacccaagtcgccgcccgtatgatgaaatttgatttcgatatcgatcctctgaaaatatgggctacttcgggccaatttacccagagtagtatggactggcacggtgccactagcaccgaccttgctgcctttcgggaccgcggcggtaaaatgattctgtatcacggaatgagcgatgccgcattctctgcactagatacagcagattattatgaacgcctgggtgccgcaatgccgggcgccgcgggctttgctcgtctgttcttggttccgggaatgaaccattgctccgggggtccaggtaccgaccgctttgatatgctaacaccgttagttgcatgggttgaacgtggggaagcccctgaccaaattagcgcctggagcggcacccccggctactttggtgtggccgcccgcactcgaccgttatgtccctatccgcagattgcgcgctataagggatcaggcgatatcaataccgaagcaaattttgcgtgtgccgctccaccgctcgagcaccaccatcaccaccactgagatccggct

SEQ ID NO: 2 - DNA, 999 bp, Artificial Sequence
  CJ MHETase Lid
tccgcgcttagctagccatggctttgtggttattaccatcgatacgaacagcactctagaccagcccagcagccgtagctcgcaacagatggccgcgcttcgtcaagttgcgagcttgaacgggaccagcagtagcccgatttacggaaaggtcgatactgcccgcatgggtgtgatgggctggtcaatggggggcggcggttcacttattagcgccgcgaacaacccgagtttaaaagcagcggcaccgcaggcgccaggatatcagttgccgaaggccggaattagtggcgcgtggaccacccagagcttagcgcccgccgccgttggcctggatgcccagggagtgccgctgattaataagagcttttctgacgcagacctccatttactgtcgcaggcgattctcggaacatgcgacgccttggatggcctggccgacggcatcgttgacaactaccgagcgtgccaagcggcttttgatccggcgactgcagccaacccagcgaatggccaagccctgcagtgcgtgggcgcaaagacagccgattgcttatcgcccgtccaagttacggcgattaaacgagcgatggccggtccggtaaatagcgcgggtacgccgttatataatagatgggcctgggacgcaggtatgagcggtcttagtggtaccacttacaatcagggttggcgcagctggtggctgggatcgtttaacagctcggcgaataacgcacaacgtgtatctggtttctcagcgcggagctggctggtggactttgctaccccgccggagccgatgcccatgacccaagtcgccgcccgtatgatgaaatttgatttcgatatcgatcctctgaaaatatgggctacttcgggccaatttacccagagtagtatggactggcacggtgccactagcaccagcagtgttaccgtgccgacgctgattttcgcgtgcgagaatgatagcattgcaccggtgaacagcagcgcgct

SEQ ID NO: 3 - DNA, 1959 bp, Artificial Sequence
  pCJ199
catatgttcgtacgcaacgccgaccgtgccaagaattgtatgcgcgcacctttacgcgtattcccactcaaggatacttttagcgcccagtgtgcgaatgtttcggtctggattaccagcagcgtaccaccgctccgcgagcgtcacatggatcgccgcgtgacgcgccgcgatttaatgcaaactcgcatcttattaatgctcattgcagccactggcgtggcagcgtgtggcggagacggtggttccacacctgccgcgcaaaatccccctttgcccctggccagtcgtgcggcttgcgaagcttttcaaggcaatagcaatagtatcgcgtggccccatcgcgcaaccgttgtggaagtggccacttggcacgaagcagagcctgcgaatgccacagcagcggcgacgcccgagcactgtgagatttccggcgccattgctcgccgcaccggaattgatggatatccttacgagattaagtttcgcttacgtatgccctcagaatggaatggtcgcttctttatggaaggggggggtggaaccaatgggtcattgagtgccgctacagggtcccttggcggtggacaaactgcgtcggccttgagtcgtaattttgcaactattgcaaccgatggtggtcatgataatgctgtcaacaataatcctgatgcgctcggcactgtcgcttttggcatggaccctcaagcgcgcattgatatgggatataattcctacgaccaggtgacccaagcgggaaaggcggccgtagcgcaattttatggccgtgccccggataaaagctattttattggctgtagcgaaggtgggcgcgaggggatgatgttgtcccaacgctttccgagtcattatgacggaattgtggcgggagcaccgggctaccaactcccaaaggcgggcatttctggcgcatggacgacgcaaagtctggcaccagcagcggtgggcgtggatgctcaaaatgtacctttgatcaataaggcgttttcggatgtcgatttacatcttctttcacgcggcattcttggtacttgcgatgccttggatggactcagcgatggaattgtgaacgacttccgtgcctgtcaagccgcctttgaccctgccactgcgttgaatcccgacaccagtcaacccttacaatgcactggtgctaagacgcctgattgcttaagtgccgcccaagtcactggcattaaacgcgccatgggtgggcctgtggacagcgccggtgcggcattatataatcgctgggcatgggaccctggcatgtcggggctcaatggcacctcttacaatcagggatggcgctcttggtggttagggagctacgcatccagcactaataatgcccaacgcgtcaatggcttttccgcccgctcttggttggttgattttgcgacgccgcctgaaccaatgccagtcacacaggttgctgctcgcatgatgaacttcaactttgacaccgacccgcctaagattcgcgcgactagtggcccctttactccatcgtctatggagtggcatggtgcaacgagcactaatcttgcggccttccgcgaccgcggtgggaagctgatgctctatcatggcatgtcagatgccgcgttctccgcattagacaccgcggattactacgagcgtttaggtgccgcgatgccgggcgccgccggcttcgcacgtcttttcttagttccaggtatgaatcattgtagtggtggacccggcactgatcgttttgatatgcttactccccttgtggcctgggtggaacgtgataaggcgccagatcaagttagcgcttgggcaggcacaccgggctatttcggcgcaaccgcccgtacacgccccctttgtccatacccccaaatcgcacgttataagggctctggtgatatcaatgccgaggcatcgtttgtgtgtgtggccccactcgag

SEQ ID NO: 4 - DNA, 1857 bp, Artificial Sequence
  pCJ200
catatgcagaccaccgtgaccaccatgctcctcgcgtccgtagcattagcggcttgcgccggaggaggttccactcctctgcctctaccgcagcagcagccgcctcagcaggaaccgccacctcctcctgttccgctagccagtcgcgccgcgtgtgaggcgctcaaagatggtaatggcgacatggtttggccgaatgccgccacggttgtagaggttgcagcctacgtgccggcaggcgttaacatcagcatggcggataacccgagcatctgtggtggcgacgaggacccgattacttccaccttcgcgttctgcgaagtatcaggcgcgattgccaagcgtactgggattgatgggtacccgtatgaaattaagtttcgcctgcgcatgcccgctgagtggaacggccgttttttcatggagggtggcagtggtacgaacggctgcctctcagcggcgaccggaagtatcggcggcggtcagatcgcctcagcgctgagtcgtaactttgcaacaattgctaccgacggaggacatgacaatgcggtgaatgataatccggatgcgctcggtaccgtcgcatttggtctcgatccccaggcacgcttagacatgggctacaactcctatgatcaggtgactcaggccggcaaagccgccgttgcacgcttttatggtcgcgcagccgacaagagctacttcatcggctgttcggagggcggccgcgagggcatgatgctgtcccagcgctttccatcacattacgatggcattgtggcgggcgcaccgggatatcagttgccgaaggccggaattagtggcgcgtggaccacccagagcttagcgcccgccgccgttggcctggatgcccagggagtgccgctgattaataagagcttttctgacgcagacctccatttactgtcgcaggcgattctcggaacatgcgacgccttggatggcctggccgacggcatcgttgacaactaccgagcgtgccaagcggcttttgatccggcgactgcagccaacccagcgaatggccaagccctgcagtgcgtgggcgcaaagacagccgattgcttatcgcccgtccaagttacggcgattaaacgagcgatggccggtccggtaaatagcgcgggtacgccgttatataatagatgggcctgggacgcaggtatgagcggtcttagtggtaccacttacaatcagggttggcgcagctggtggctgggatcgtttaacagctcggcgaataacgcacaacgtgtatctggtttctcagcgcggagctggctggtggactttgctaccccgccggagccgatgcccatgacccaagtcgccgcccgtatgatgaaatttgatttcgatatcgatcctctgaaaatatgggctacttcgggccaatttacccagagtagtatggactggcacggtgccactagcaccgaccttgctgcctttcgggaccgcggcggtaaaatgattctgtatcacggaatgagcgatgccgcattctctgcactagatacagcagattattatgaacgcctgggtgccgcaatgccgggcgccgcgggctttgctcgtctgttcttggttccgggaatgaaccattgctccgggggtccaggtaccgaccgctttgatatgctaacaccgttagttgcatgggttgaacgtggggaagcccctgaccaaattagcgcctggagcggcacccccggctactttggtgtggccgcccgcactcgaccgttatgtccctatccgcagattgcgcgctataagggatcaggcgatatcaataccgaagcaaattttgcgtgtgccgctccaccgctcgag

SEQ ID NO: 5 - DNA, 1785 bp, Artificial Sequence
  pCJ207
catatgaagtctagcattccgattagcgtaggtatgctggctaccgctctgatttccggttgcggtagcgttccggataacaccagcaatgaaccgacggttccgctggcatccaaagaattgtgcgagggcatcgcgtctggtgcgaccaaagtaaactggccgaaccagaacaccgtcgtaaaagcttcagtttggcacgctgttaccccggcaaccgccaacgccccggaactgccggaacattgcgaggtcactggctctatcaaccagcgtactggcgtggacggctatccgtatgaaatcaaaatgcgtctgcgcatgccggcagattggaacggccgtttcttcatggaaggcggtggaggtactaacgggagcctgtctgccgctctgggttcgctgggcggtggtcagaccagcaatgctctgagccgtcgtttcgctaccgtttctaccgatggtggtcatgataacgcagtgaacaacaatccggcggcgctgggttcggtcgctttcggcatggacccgcaggctcgcctggatcatggttacaactcatacgatcaggttaccctggcgggcaagtcagcagtaagcactttttacgggcgcggcccggacaaatcatacttcatcggctgttccgaaggcggtcgtgagggcatgatgttcagccagcgtttcccggcgcactatgacggcatcgtcgccggtgcaccgggctaccagctgccgaaagcaggcatcagcggtgcatggacaacgcaatctctggcaccagcggccgttggtgttgacccggacggtgcaccgctggtgaacaaatccttcagcgatccggacctgtatctgctgactcaggcaatcctgggcagctgcgacgctctggacggtctggctgacggtatcgtcggcaattattccgcgtgtcagtcgctgtttgacccgtctaccgccttgaaccctgcgacgggacaaccactgcgttgcacgggcgctaagaccgacgactgcctaagcccggttcaggtggatgcgatcaaacgtgctatgtccggtccagttgatactgccggaaccgccctgtataacaaatggccgtgggataccggtatgtcgggcctgaacggcaccacttatttccagggctggcgcagctggtggctgggctcctacgacagctctactaacaacgcgcagcgtgttaacggcagcagcgcacgctcttggctggtagatttcgctactccgccggaacctgtaccgctgaaccaggtggccactcgtatgatgaactttgattttgatgttgacccgccgaaaatctttgctacctctggtctctttacccagccgtccatgcaatggcacggtgccacctcaaccgatctgaacgcttttcgctctcgcggtggcaagctgatgctgtaccacggcatggctgacgcggcattcagcgcactggataccattgcttattatgagcgcctgagcaccgcaatgccttccgtgtccgacttttctcgcctgtttctggtgcctggtatggggcactgttccggcggtccgggcaccgatcgctttgatatgctgactccgctggtggcgtgggttgagaacggtactgcaccggctcgcgtcgaagcgtcgtcctccactccgggttacttcggtgtttcggcccgcagccgccccctgtgcccgcatccgcagattgcacgttataccgggtccggcgacattaacgaagccaccaactttgtatgcggtaacccgctcgag

SEQ ID NO: 6 - DNA, 14 bp, Artificial Sequence
  Primer CJ756
tcggagggcggccg

SEQ ID NO: 7 - DNA, 27 bp, Artificial Sequence
  primer CJ757
ggcgccgatgaagtagctcttgtcggc

SEQ ID NO: 8 - DNA, 18 bp, Artificial Sequence
  primer CJ758
tccgggggtccaggtacc

SEQ ID NO: 9 - DNA, 30 bp, Artificial Sequence
  primer CJ759
ggcatggttcattcccggaaccaagaacag

SEQ ID NO: 10 - DNA, 27 bp, Artificial Sequence
  primer CJ760
ccagccgatgaagtagctcttgtcggc

SEQ ID NO: 11 - DNA, 30 bp, Artificial Sequence
  primer CJ761
gctatggttcattcccggaaccaagaacag

SEQ ID NO: 12 - DNA, 27 bp, Artificial Sequence
  primer CJ762
gtggccgatgaagtagctcttgtcggc

SEQ ID NO: 13 - DNA, 30 bp, Artificial Sequence
  primer CJ763
gaaatggttcattcccggaaccaagaacag

SEQ ID NO: 14 - DNA, 16 bp, Artificial Sequence
  primer CJ764
tcaatggggggcggcg

SEQ ID NO: 15 - DNA, 22 bp, Artificial Sequence
  primer CJ765
gcagcccatcacacccatgcgg

SEQ ID NO: 16 - DNA, 21 bp, Artificial Sequence
  primer CJ766
tgtgccaactctgggaacagc

SEQ ID NO: 17 - DNA, 27 bp, Artificial Sequence
  primer CJ767
gcagtggctaccgccgttaatttccag

SEQ ID NO: 18 - DNA, 14 bp, Artificial Sequence
  primer CJ768
gagggcggccgcga

SEQ ID NO: 19 - DNA, 28 bp, Artificial Sequence
  primer CJ769
ggcacagccgatgaagtagctcttgtcg

SEQ ID NO: 20 - DNA, 16 bp, Artificial Sequence
  primer CJ770
tgtggcggagacggtg

SEQ ID NO: 21 - DNA, 43 bp, Artificial Sequence
  primer CJ771
catatgtatatctccttcttaaagttaaacaaaattatttcta

SEQ ID NO: 22 - DNA, 16 bp, Artificial Sequence
  primer CJ772
tgcggtagcgttccgg

SEQ ID NO: 23 - DNA, 25 bp, Artificial Sequence
  primer CJ773
ggcggtacgaacggctctctctcag

SEQ ID NO: 24 - DNA, 22 bp, Artificial Sequence
  primer CJ774
gccaccctccatgaaaaaacgg

SEQ ID NO: 25 - DNA, 34 bp, Artificial Sequence
  primer CJ775
atctctgcactagatacagcagattattatgaac

SEQ ID NO: 26 - DNA, 18 bp, Artificial Sequence
  primer CJ776
tgcggcatcgctcattcc

SEQ ID NO: 27 - DNA, 13 bp, Artificial Sequence
  primer CJ777
ggcggccgcgagg

SEQ ID NO: 28 - DNA, 30 bp, Artificial Sequence
  primer CJ778
ggtcgaacagccgatgaagtagctcttgtc

SEQ ID NO: 29 - DNA, 25 bp, Artificial Sequence
  primer CJ779
tgcatgagcgatgccgcattctctg

SEQ ID NO: 30 - DNA, 27 bp, Artificial Sequence
  primer CJ780
gtgatacagaatcattttaccgccgcg

SEQ ID NO: 31 - DNA, 18 bp, Artificial Sequence
  primer CJ781
gggggtccaggtaccgac

SEQ ID NO: 32 - DNA, 25 bp, Artificial Sequence
  primer CJ782
gcagcaatggttcattcccggaacc

SEQ ID NO: 33 - DNA, 32 bp, Artificial Sequence
  primer CJ787
caaccaacttcgaccttgctgcctttcgggac

SEQ ID NO: 34 - DNA, 23 bp, Artificial Sequence
  primer CJ788
aagagtcccacggtgcgcccgcc

SEQ ID NO: 35 - DNA, 2649 bp, Artificial Sequence
  DNA sequence for MHETase - 8 aa linker - PETase
atgcagaccaccgtgaccaccatgctcctcgcgtccgtagcattagcggcttgcgccggaggaggttccactcctctgcctctaccgcagcagcagccgcctcagcaggaaccgccacctcctcctgttccgctagccagtcgcgccgcgtgtgaggcgctcaaagatggtaatggcgacatggtttggccgaatgccgccacggttgtagaggttgcagcctggcgtgatgcagcaccggccacggcatcagccgcagccctgccggagcattgcgaagtatcaggcgcgattgccaagcgtactgggattgatgggtacccgtatgaaattaagtttcgcctgcgcatgcccgctgagtggaacggccgttttttcatggagggtggcagtggtacgaacggctctctctcagcggcgaccggaagtatcggcggcggtcagatcgcctcagcgctgagtcgtaactttgcaacaattgctaccgacggaggacatgacaatgcggtgaatgataatccggatgcgctcggtaccgtcgcatttggtctcgatccccaggcacgcttagacatgggctacaactcctatgatcaggtgactcaggccggcaaagccgccgttgcacgcttttatggtcgcgcagccgacaagagctacttcatcggctgttcggagggcggccgcgagggcatgatgctgtcccagcgctttccatcacattacgatggcattgtggcgggcgcaccgggatatcagttgccgaaggccggaattagtggcgcgtggaccacccagagcttagcgcccgccgccgttggcctggatgcccagggagtgccgctgattaataagagcttttctgacgcagacctccatttactgtcgcaggcgattctcggaacatgcgacgccttggatggcctggccgacggcatcgttgacaactaccgagcgtgccaagcggcttttgatccggcgactgcagccaacccagcgaatggccaagccctgcagtgcgtgggcgcaaagacagccgattgcttatcgcccgtccaagttacggcgattaaacgagcgatggccggtccggtaaatagcgcgggtacgccgttatataatagatgggcctgggacgcaggtatgagcggtcttagtggtaccacttacaatcagggttggcgcagctggtggctgggatcgtttaacagctcggcgaataacgcacaacgtgtatctggtttctcagcgcggagctggctggtggactttgctaccccgccggagccgatgcccatgacccaagtcgccgcccgtatgatgaaatttgatttcgatatcgatcctctgaaaatatgggctacttcgggccaatttacccagagtagtatggactggcacggtgccactagcaccgaccttgctgcctttcgggaccgcggcggtaaaatgattctgtatcacggaatgagcgatgccgcattctctgcactagatacagcagattattatgaacgcctgggtgccgcaatgccgggcgccgcgggctttgctcgtctgttcttggttccgggaatgaaccattgctccgggggtccaggtaccgaccgctttgatatgctaacaccgttagttgcatgggttgaacgtggggaagcccctgaccaaattagcgcctggagcggcacccccggctactttggtgtggccgcccgcactcgaccgttatgtccctatccgcagattgcgcgctataagggatcaggcgatatcaataccgaagcaaattttgcgtgtgccgctccaccgggtggtggttctggtggttctggtcagaccaatccgtatgcgcgcggccccaaccctaccgccgcctcgttggaagccagcgcgggaccctttaccgttcgtagctttaccgttagccgtccgtccggatatggtgcagggaccgtctattacccaaccaatgcaggcggcaccgttggcgcgattgcaatcgtccccgggtacaccgcgcgtcaaagcagcattaagtggtggggtccgcgcttagctagccatggctttgtggttattaccatcgatacgaacagcactctagaccagcccagcagccgtagctcgcaacagatggccgcgcttcgtcaagttgcgagcttgaacgggaccagcagtagcccgatttacggaaaggtcgatactgcccgcatgggtgtgatgggctggtcaatggggggcggcggttcacttattagcgccgcgaacaacccgagtttaaaagcagcggcaccgcaggcgccatgggactcttcaaccaacttcagcagtgttaccgtgccgacgctgattttcgcgtgcgagaatgatagcattgcaccggtgaacagcagcgcgctgccgatttatgatagcatgtcccgcaacgcaaaacagtttctggaaattaacggcggtagccactcttgtgccaactctgggaacagcaaccaggcactgatcggaaaaaaaggggttgcatggatgaaacgattcatggataatgacacccgttactcaaccttcgcctgtgagaatcccaacagcacacgcgtgtcggattttcgcaccgcgaactgttccctcgagcaccaccatcaccaccactga

SEQ ID NO: 36 - protein, 882 aa, Artificial Sequence
  Amino acid sequence for MHETase - 8 aa linker - PETase
MQTTVTTMLLASVALAACAGGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRNFATIATDGGHDNAVNDNPDALGTVAFGLDPQARLDMGYNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPPGGGSGGSGQTNPYARGPNPTAASLEASAGPFTVRSFTVSRPSGYGAGTVYYPTNAGGTVGAIAIVPGYTARQSSIKWWGPRLASHGFVVITIDTNSTLDQPSSRSSQQMAALRQVASLNGTSSSPIYGKVDTARMGVMGWSMGGGGSLISAANNPSLKAAAPQAPWDSSTNFSSVTVPTLIFACENDSIAPVNSSALPIYDSMSRNAKQFLEINGGSHSCANSGNSNQALIGKKGVAWMKRFMDNDTRYSTFACENPNSTRVSDFRTANCSLEHHHHHH

SEQ ID NO: 37 - DNA, 2661 bp, Artificial Sequence
  DNA sequence for MHETase - 12 aa linker - PETase
atgcagaccaccgtgaccaccatgctcctcgcgtccgtagcattagcggcttgcgccggaggaggttccactcctctgcctctaccgcagcagcagccgcctcagcaggaaccgccacctcctcctgttccgctagccagtcgcgccgcgtgtgaggcgctcaaagatggtaatggcgacatggtttggccgaatgccgccacggttgtagaggttgcagcctggcgtgatgcagcaccggccacggcatcagccgcagccctgccggagcattgcgaagtatcaggcgcgattgccaagcgtactgggattgatgggtacccgtatgaaattaagtttcgcctgcgcatgcccgctgagtggaacggccgttttttcatggagggtggcagtggtacgaacggctctctctcagcggcgaccggaagtatcggcggcggtcagatcgcctcagcgctgagtcgtaactttgcaacaattgctaccgacggaggacatgacaatgcggtgaatgataatccggatgcgctcggtaccgtcgcatttggtctcgatccccaggcacgcttagacatgggctacaactcctatgatcaggtgactcaggccggcaaagccgccgttgcacgcttttatggtcgcgcagccgacaagagctacttcatcggctgttcggagggcggccgcgagggcatgatgctgtcccagcgctttccatcacattacgatggcattgtggcgggcgcaccgggatatcagttgccgaaggccggaattagtggcgcgtggaccacccagagcttagcgcccgccgccgttggcctggatgcccagggagtgccgctgattaataagagcttttctgacgcagacctccatttactgtcgcaggcgattctcggaacatgcgacgccttggatggcctggccgacggcatcgttgacaactaccgagcgtgccaagcggcttttgatccggcgactgcagccaacccagcgaatggccaagccctgcagtgcgtgggcgcaaagacagccgattgcttatcgcccgtccaagttacggcgattaaacgagcgatggccggtccggtaaatagcgcgggtacgccgttatataatagatgggcctgggacgcaggtatgagcggtcttagtggtaccacttacaatcagggttggcgcagctggtggctgggatcgtttaacagctcggcgaataacgcacaacgtgtatctggtttctcagcgcggagctggctggtggactttgctaccccgccggagccgatgcccatgacccaagtcgccgcccgtatgatgaaatttgatttcgatatcgatcctctgaaaatatgggctacttcgggccaatttacccagagtagtatggactggcacggtgccactagcaccgaccttgctgcctttcgggaccgcggcggtaaaatgattctgtatcacggaatgagcgatgccgcattctctgcactagatacagcagattattatgaacgcctgggtgccgcaatgccgggcgccgcgggctttgctcgtctgttcttggttccgggaatgaaccattgctccgggggtccaggtaccgaccgctttgatatgctaacaccgttagttgcatgggttgaacgtggggaagcccctgaccaaattagcgcctggagcggcacccccggctactttggtgtggccgcccgcactcgaccgttatgtccctatccgcagattgcgcgctataagggatcaggcgatatcaataccgaagcaaattttgcgtgtgccgctccaccgggtggtggttctggtggttctggtggtggttctggtcagaccaatccgtatgcgcgcggccccaaccctaccgccgcctcgttggaagccagcgcgggaccctttaccgttcgtagctttaccgttagccgtccgtccggatatggtgcagggaccgtctattacccaaccaatgcaggcggcaccgttggcgcgattgcaatcgtccccgggtacaccgcgcgtcaaagcagcattaagtggtggggtccgcgcttagctagccatggctttgtggttattaccatcgatacgaacagcactctagaccagcccagcagccgtagctcgcaacagatggccgcgcttcgtcaagttgcgagcttgaacgggaccagcagtagcccgatttacggaaaggtcgatactgcccgcatgggtgtgatgggctggtcaatggggggcggcggttcacttattagcgccgcgaacaacccgagtttaaaagcagcggcaccgcaggcgccatgggactcttcaaccaacttcagcagtgttaccgtgccgacgctgattttcgcgtgcgagaatgatagcattgcaccggtgaacagcagcgcgctgccgatttatgatagcatgtcccgcaacgcaaaacagtttctggaaattaacggcggtagccactcttgtgccaactctgggaacagcaaccaggcactgatcggaaaaaaaggggttgcatggatgaaacgattcatggataatgacacccgttactcaaccttcgcctgtgagaatcccaacagcacacgcgtgtcggattttcgcaccgcgaactgttccctcgagcaccaccatcaccaccactga

SEQ ID NO: 38 - protein, 886 aa, Artificial Sequence
  Amino Acid sequence for MHETase - 12 aa linker - PETase
MQTTVTTMLLASVALAACAGGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRNFATIATDGGHDNAVNDNPDALGTVAFGLDPQARLDMGYNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPPGGGSGGSGGGSGQTNPYARGPNPTAASLEASAGPFTVRSFTVSRPSGYGAGTVYYPTNAGGTVGAIAIVPGYTARQSSIKWWGPRLASHGFVVITIDTNSTLDQPSSRSSQQMAALRQVASLNGTSSSPIYGKVDTARMGVMGWSMGGGGSLISAANNPSLKAAAPQAPWDSSTNFSSVTVPTLIFACENDSIAPVNSSALPIYDSMSRNAKQFLEINGGSHSCANSGNSNQALIGKKGVAWMKRFMDNDTRYSTFACENPNSTRVSDFRTANCSLEHHHHHH

SEQ ID NO: 39 - DNA, 2685 bp, Artificial Sequence
  DNA sequence for MHETase - 20 aa linker - PETase
atgcagaccaccgtgaccaccatgctcctcgcgtccgtagcattagcggcttgcgccggaggaggttccactcctctgcctctaccgcagcagcagccgcctcagcaggaaccgccacctcctcctgttccgctagccagtcgcgccgcgtgtgaggcgctcaaagatggtaatggcgacatggtttggccgaatgccgccacggttgtagaggttgcagcctggcgtgatgcagcaccggccacggcatcagccgcagccctgccggagcattgcgaagtatcaggcgcgattgccaagcgtactgggattgatgggtacccgtatgaaattaagtttcgcctgcgcatgcccgctgagtggaacggccgttttttcatggagggtggcagtggtacgaacggctctctctcagcggcgaccggaagtatcggcggcggtcagatcgcctcagcgctgagtcgtaactttgcaacaattgctaccgacggaggacatgacaatgcggtgaatgataatccggatgcgctcggtaccgtcgcatttggtctcgatccccaggcacgcttagacatgggctacaactcctatgatcaggtgactcaggccggcaaagccgccgttgcacgcttttatggtcgcgcagccgacaagagctacttcatcggctgttcggagggcggccgcgagggcatgatgctgtcccagcgctttccatcacattacgatggcattgtggcgggcgcaccgggatatcagttgccgaaggccggaattagtggcgcgtggaccacccagagcttagcgcccgccgccgttggcctggatgcccagggagtgccgctgattaataagagcttttctgacgcagacctccatttactgtcgcaggcgattctcggaacatgcgacgccttggatggcctggccgacggcatcgttgacaactaccgagcgtgccaagcggcttttgatccggcgactgcagccaacccagcgaatggccaagccctgcagtgcgtgggcgcaaagacagccgattgcttatcgcccgtccaagttacggcgattaaacgagcgatggccggtccggtaaatagcgcgggtacgccgttatataatagatgggcctgggacgcaggtatgagcggtcttagtggtaccacttacaatcagggttggcgcagctggtggctgggatcgtttaacagctcggcgaataacgcacaacgtgtatctggtttctcagcgcggagctggctggtggactttgctaccccgccggagccgatgcccatgacccaagtcgccgcccgtatgatgaaatttgatttcgatatcgatcctctgaaaatatgggctacttcgggccaatttacccagagtagtatggactggcacggtgccactagcaccgaccttgctgcctttcgggaccgcggcggtaaaatgattctgtatcacggaatgagcgatgccgcattctctgcactagatacagcagattattatgaacgcctgggtgccgcaatgccgggcgccgcgggctttgctcgtctgttcttggttccgggaatgaaccattgctccgggggtccaggtaccgaccgctttgatatgctaacaccgttagttgcatgggttgaacgtggggaagcccctgaccaaattagcgcctggagcggcacccccggctactttggtgtggccgcccgcactcgaccgttatgtccctatccgcagattgcgcgctataagggatcaggcgatatcaataccgaagcaaattttgcgtgtgccgctccaccgggtggtggttctggtggttctggtggtggttctggtggtggtggttctggtggttctggtcagaccaatccgtatgcgcgcggccccaaccctaccgccgcctcgttggaagccagcgcgggaccctttaccgttcgtagctttaccgttagccgtccgtccggatatggtgcagggaccgtctattacccaaccaatgcaggcggcaccgttggcgcgattgcaatcgtccccgggtacaccgcgcgtcaaagcagcattaagtggtggggtccgcgcttagctagccatggctttgtggttattaccatcgatacgaacagcactctagaccagcccagcagccgtagctcgcaacagatggccgcgcttcgtcaagttgcgagcttgaacgggaccagcagtagcccgatttacggaaaggtcgatactgcccgcatgggtgtgatgggctggtcaatggggggcggcggttcacttattagcgccgcgaacaacccgagtttaaaagcagcggcaccgcaggcgccatgggactcttcaaccaacttcagcagtgttaccgtgccgacgctgattttcgcgtgcgagaatgatagcattgcaccggtgaacagcagcgcgctgccgatttatgatagcatgtcccgcaacgcaaaacagtttctggaaattaacggcggtagccactcttgtgccaactctgggaacagcaaccaggcactgatcggaaaaaaaggggttgcatggatgaaacgattcatggataatgacacccgttactcaaccttcgcctgtgagaatcccaacagcacacgcgtgtcggattttcgcaccgcgaactgttccctcgagcaccaccatcaccaccactga

SEQ ID NO: 40 - protein, 894 aa, Artificial Sequence
  Amino acid sequence for MHETase - 20 aa linker - PETase
MQTTVTTMLLASVALAACAGGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRNFATIATDGGHDNAVNDNPDALGTVAFGLDPQARLDMGYNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPPGGGSGGSGGGSGGGGSGGSGQTNPYARGPNPTAASLEASAGPFTVRSFTVSRPSGYGAGTVYYPTNAGGTVGAIAIVPGYTARQSSIKWWGPRLASHGFVVITIDTNSTLDQPSSRSSQQMAALRQVASLNGTSSSPIYGKVDTARMGVMGWSMGGGGSLISAANNPSLKAAAPQAPWDSSTNFSSVTVPTLIFACENDSIAPVNSSALPIYDSMSRNAKQFLEINGGSHSCANSGNSNQALIGKKGVAWMKRFMDNDTRYSTFACENPNSTRVSDFRTANCSLEHHHHHH

SEQ ID NO: 41 - DNA, 2679 bp, Artificial Sequence
  DNA sequence for PETase - 8 aa linker - MHETase
atgaacttcccccgtgcctcgcgccttatgcaggctgctgtgctgggcggccttatggccgtttccgcagcggccaccgcgcagaccaatccgtatgcgcgcggccccaaccctaccgccgcctcgttggaagccagcgcgggaccctttaccgttcgtagctttaccgttagccgtccgtccggatatggtgcagggaccgtctattacccaaccaatgcaggcggcaccgttggcgcgattgcaatcgtccccgggtacaccgcgcgtcaaagcagcattaagtggtggggtccgcgcttagctagccatggctttgtggttattaccatcgatacgaacagcactctagaccagcccagcagccgtagctcgcaacagatggccgcgcttcgtcaagttgcgagcttgaacgggaccagcagtagcccgatttacggaaaggtcgatactgcccgcatgggtgtgatgggctggtcaatggggggcggcggttcacttattagcgccgcgaacaacccgagtttaaaagcagcggcaccgcaggcgccatgggactcttcaaccaacttcagcagtgttaccgtgccgacgctgattttcgcgtgcgagaatgatagcattgcaccggtgaacagcagcgcgctgccgatttatgatagcatgtcccgcaacgcaaaacagtttctggaaattaacggcggtagccactcttgtgccaactctgggaacagcaaccaggcactgatcggaaaaaaaggggttgcatggatgaaacgattcatggataatgacacccgttactcaaccttcgcctgtgagaatcccaacagcacacgcgtgtcggattttcgcaccgcgaactgttccggtggtggttctggtggttctggttgcgccggaggaggttccactcctctgcctctaccgcagcagcagccgcctcagcaggaaccgccacctcctcctgttccgctagccagtcgcgccgcgtgtgaggcgctcaaagatggtaatggcgacatggtttggccgaatgccgccacggttgtagaggttgcagcctggcgtgatgcagcaccggccacggcatcagccgcagccctgccggagcattgcgaagtatcaggcgcgattgccaagcgtactgggattgatgggtacccgtatgaaattaagtttcgcctgcgcatgcccgctgagtggaacggccgttttttcatggagggtggcagtggtacgaacggctctctctcagcggcgaccggaagtatcggcggcggtcagatcgcctcagcgctgagtcgtaactttgcaacaattgctaccgacggaggacatgacaatgcggtgaatgataatccggatgcgctcggtaccgtcgcatttggtctcgatccccaggcacgcttagacatgggctacaactcctatgatcaggtgactcaggccggcaaagccgccgttgcacgcttttatggtcgcgcagccgacaagagctacttcatcggctgttcggagggcggccgcgagggcatgatgctgtcccagcgctttccatcacattacgatggcattgtggcgggcgcaccgggatatcagttgccgaaggccggaattagtggcgcgtggaccacccagagcttagcgcccgccgccgttggcctggatgcccagggagtgccgctgattaataagagcttttctgacgcagacctccatttactgtcgcaggcgattctcggaacatgcgacgccttggatggcctggccgacggcatcgttgacaactaccgagcgtgccaagcggcttttgatccggcgactgcagccaacccagcgaatggccaagccctgcagtgcgtgggcgcaaagacagccgattgcttatcgcccgtccaagttacggcgattaaacgagcgatggccggtccggtaaatagcgcgggtacgccgttatataatagatgggcctgggacgcaggtatgagcggtcttagtggtaccacttacaatcagggttggcgcagctggtggctgggatcgtttaacagctcggcgaataacgcacaacgtgtatctggtttctcagcgcggagctggctggtggactttgctaccccgccggagccgatgcccatgacccaagtcgccgcccgtatgatgaaatttgatttcgatatcgatcctctgaaaatatgggctacttcgggccaatttacccagagtagtatggactggcacggtgccactagcaccgaccttgctgcctttcgggaccgcggcggtaaaatgattctgtatcacggaatgagcgatgccgcattctctgcactagatacagcagattattatgaacgcctgggtgccgcaatgccgggcgccgcgggctttgctcgtctgttcttggttccgggaatgaaccattgctccgggggtccaggtaccgaccgctttgatatgctaacaccgttagttgcatgggttgaacgtggggaagcccctgaccaaattagcgcctggagcggcacccccggctactttggtgtggccgcccgcactcgaccgttatgtccctatccgcagattgcgcgctataagggatcaggcgatatcaataccgaagcaaattttgcgtgtgccgctccaccgctcgagcaccaccatcaccaccactga

SEQ ID NO: 42 - DNA, 2691 bp, Artificial Sequence
  DNA sequence for PETase - 12 aa linker - MHETase
atgaacttcccccgtgcctcgcgccttatgcaggctgctgtgctgggcggccttatggccgtttccgcagcggccaccgcgcagaccaatccgtatgcgcgcggccccaaccctaccgccgcctcgttggaagccagcgcgggaccctttaccgttcgtagctttaccgttagccgtccgtccggatatggtgcagggaccgtctattacccaaccaatgcaggcggcaccgttggcgcgattgcaatcgtccccgggtacaccgcgcgtcaaagcagcattaagtggtggggtccgcgcttagctagccatggctttgtggttattaccatcgatacgaacagcactctagaccagcccagcagccgtagctcgcaacagatggccgcgcttcgtcaagttgcgagcttgaacgggaccagcagtagcccgatttacggaaaggtcgatactgcccgcatgggtgtgatgggctggtcaatggggggcggcggttcacttattagcgccgcgaacaacccgagtttaaaagcagcggcaccgcaggcgccatgggactcttcaaccaacttcagcagtgttaccgtgccgacgctgattttcgcgtgcgagaatgatagcattgcaccggtgaacagcagcgcgctgccgatttatgatagcatgtcccgcaacgcaaaacagtttctggaaattaacggcggtagccactcttgtgccaactctgggaacagcaaccaggcactgatcggaaaaaaaggggttgcatggatgaaacgattcatggataatgacacccgttactcaaccttcgcctgtgagaatcccaacagcacacgcgtgtcggattttcgcaccgcgaactgttccggtggtggttctggtggttctggtggtggttctggttgcgccggaggaggttccactcctctgcctctaccgcagcagcagccgcctcagcaggaaccgccacctcctcctgttccgctagccagtcgcgccgcgtgtgaggcgctcaaagatggtaatggcgacatggtttggccgaatgccgccacggttgtagaggttgcagcctggcgtgatgcagcaccggccacggcatcagccgcagccctgccggagcattgcgaagtatcaggcgcgattgccaagcgtactgggattgatgggtacccgtatgaaattaagtttcgcctgcgcatgcccgctgagtggaacggccgttttttcatggagggtggcagtggtacgaacggctctctctcagcggcgaccggaagtatcggcggcggtcagatcgcctcagcgctgagtcgtaactttgcaacaattgctaccgacggaggacatgacaatgcggtgaatgataatccggatgcgctcggtaccgtcgcatttggtctcgatccccaggcacgcttagacatgggctacaactcctatgatcaggtgactcaggccggcaaagccgccgttgcacgcttttatggtcgcgcagccgacaagagctacttcatcggctgttcggagggcggccgcgagggcatgatgctgtcccagcgctttccatcacattacgatggcattgtggcgggcgcaccgggatatcagttgccgaaggccggaattagtggcgcgtggaccacccagagcttagcgcccgccgccgttggcctggatgcccagggagtgccgctgattaataagagcttttctgacgcagacctccatttactgtcgcaggcgattctcggaacatgcgacgccttggatggcctggccgacggcatcgttgacaactaccgagcgtgccaagcggcttttgatccggcgactgcagccaacccagcgaatggccaagccctgcagtgcgtgggcgcaaagacagccgattgcttatcgcccgtccaagttacggcgattaaacgagcgatggccggtccggtaaatagcgcgggtacgccgttatataatagatgggcctgggacgcaggtatgagcggtcttagtggtaccacttacaatcagggttggcgcagctggtggctgggatcgtttaacagctcggcgaataacgcacaacgtgtatctggtttctcagcgcggagctggctggtggactttgctaccccgccggagccgatgcccatgacccaagtcgccgcccgtatgatgaaatttgatttcgatatcgatcctctgaaaatatgggctacttcgggccaatttacccagagtagtatggactggcacggtgccactagcaccgaccttgctgcctttcgggaccgcggcggtaaaatgattctgtatcacggaatgagcgatgccgcattctctgcactagatacagcagattattatgaacgcctgggtgccgcaatgccgggcgccgcgggctttgctcgtctgttcttggttccgggaatgaaccattgctccgggggtccaggtaccgaccgctttgatatgctaacaccgttagttgcatgggttgaacgtggggaagcccctgaccaaattagcgcctggagcggcacccccggctactttggtgtggccgcccgcactcgaccgttatgtccctatccgcagattgcgcgctataagggatcaggcgatatcaataccgaagcaaattttgcgtgtgccgctccaccgctcgagcaccaccatcaccaccactga

SEQ ID NO: 43 - DNA, 2715 bp, Artificial Sequence
  DNA sequence for PETase - 20 aa linker - MHETase
atgaacttcccccgtgcctcgcgccttatgcaggctgctgtgctgggcggccttatggccgtttccgcagcggccaccgcgcagaccaatccgtatgcgcgcggccccaaccctaccgccgcctcgttggaagccagcgcgggaccctttaccgttcgtagctttaccgttagccgtccgtccggatatggtgcagggaccgtctattacccaaccaatgcaggcggcaccgttggcgcgattgcaatcgtccccgggtacaccgcgcgtcaaagcagcattaagtggtggggtccgcgcttagctagccatggctttgtggttattaccatcgatacgaacagcactctagaccagcccagcagccgtagctcgcaacagatggccgcgcttcgtcaagttgcgagcttgaacgggaccagcagtagcccgatttacggaaaggtcgatactgcccgcatgggtgtgatgggctggtcaatggggggcggcggttcacttattagcgccgcgaacaacccgagtttaaaagcagcggcaccgcaggcgccatgggactcttcaaccaacttcagcagtgttaccgtgccgacgctgattttcgcgtgcgagaatgatagcattgcaccggtgaacagcagcgcgctgccgatttatgatagcatgtcccgcaacgcaaaacagtttctggaaattaacggcggtagccactcttgtgccaactctgggaacagcaaccaggcactgatcggaaaaaaaggggttgcatggatgaaacgattcatggataatgacacccgttactcaaccttcgcctgtgagaatcccaacagcacacgcgtgtcggattttcgcaccgcgaactgttccggtggtggttctggtggttctggtggtggttctggtggtggtggttctggtggttctggttgcgccggaggaggttccactcctctgcctctaccgcagcagcagccgcctcagcaggaaccgccacctcctcctgttccgctagccagtcgcgccgcgtgtgaggcgctcaaagatggtaatggcgacatggtttggccgaatgccgccacggttgtagaggttgcagcctggcgtgatgcagcaccggccacggcatcagccgcagccctgccggagcattgcgaagtatcaggcgcgattgccaagcgtactgggattgatgggtacccgtatgaaattaagtttcgcctgcgcatgcccgctgagtggaacggccgttttttcatggagggtggcagtggtacgaacggctctctctcagcggcgaccggaagtatcggcggcggtcagatcgcctcagcgctgagtcgtaactttgcaacaattgctaccgacggaggacatgacaatgcggtgaatgataatccggatgcgctcggtaccgtcgcatttggtctcgatccccaggcacgcttagacatgggctacaactcctatgatcaggtgactcaggccggcaaagccgccgttgcacgcttttatggtcgcgcagccgacaagagctacttcatcggctgttcggagggcggccgcgagggcatgatgctgtcccagcgctttccatcacattacgatggcattgtggcgggcgcaccgggatatcagttgccgaaggccggaattagtggcgcgtggaccacccagagcttagcgcccgccgccgttggcctggatgcccagggagtgccgctgattaataagagcttttctgacgcagacctccatttactgtcgcaggcgattctcggaacatgcgacgccttggatggcctggccgacggcatcgttgacaactaccgagcgtgccaagcggcttttgatccggcgactgcagccaacccagcgaatggccaagccctgcagtgcgtgggcgcaaagacagccgattgcttatcgcccgtccaagttacggcgattaaacgagcgatggccggtccggtaaatagcgcgggtacgccgttatataatagatgggcctgggacgcaggtatgagcggtcttagtggtaccacttacaatcagggttggcgcagctggtggctgggatcgtttaacagctcggcgaataacgcacaacgtgtatctggtttctcagcgcggagctggctggtggactttgctaccccgccggagccgatgcccatgacccaagtcgccgcccgtatgatgaaatttgatttcgatatcgatcctctgaaaatatgggctacttcgggccaatttacccagagtagtatggactggcacggtgccactagcaccgaccttgctgcctttcgggaccgcggcggtaaaatgattctgtatcacggaatgagcgatgccgcattctctgcactagatacagcagattattatgaacgcctgggtgccgcaatgccgggcgccgcgggctttgctcgtctgttcttggttccgggaatgaaccattgctccgggggtccaggtaccgaccgctttgatatgctaacaccgttagttgcatgggttgaacgtggggaagcccctgaccaaattagcgcctggagcggcacccccggctactttggtgtggccgcccgcactcgaccgttatgtccctatccgcagattgcgcgctataagggatcaggcgatatcaataccgaagcaaattttgcgtgtgccgctccaccgctcgagcaccaccatcaccaccactga